(19) United States
(12) Patent Application Publication
(10) Pub. No.: US 2024/0182981 A1
(43) Pub. Date: Jun. 6, 2024

(54) IDENTIFICATION AND DESIGN OF CANCER THERAPIES BASED ON RNA SEQUENCING

(71) Applicant: Genomic Expression Inc., Beverly, MA (US)

(72) Inventors: Morten Lorentz PEDERSEN, Beverly, MA (US); Gitte Laurette PEDERSEN, Montauk, NY (US); Tanya Sharlene KANIGAN, Shelburne, VT (US)

(21) Appl. No.: 18/503,844

(22) Filed: Nov. 7, 2023

Related U.S. Application Data
(63) Continuation of application No. PCT/US22/28582, filed on May 10, 2022.
(60) Provisional application No. 63/187,210, filed on May 11, 2021.

(51) Int. Cl. C12Q 1/6886; C12Q 1/6874; G16B 25/10

(52) U.S. Cl. CPC C12Q 1/6886; C12Q 1/6874; G16B 25/10; C12Q 2600/158; C12Q 2600/16

(57) ABSTRACT

Provided herein are compositions and methods for quantifying the RNA transcription level of one or more genes in biological samples. Such methods can be useful for detecting aberrantly expressed genes, and diagnosing various diseases and conditions, such as a cancer. The methods can also include providing a wellness recommendations, including, for example, a treatment recommendation, suitable therapeutic agent, combination therapy, or clinical trial.

### CROSS REFERENCE

This application is a continuation of International Application No. PCT/US22/28582 filed on May 10, 2022, which claims the benefit of U.S. Provisional Patent Application No. 63/187,210, filed May 11, 2021, each of which is incorporated herein by reference in its entirety.

### BACKGROUND

Cancer is a highly heterogeneous disease and even the best cancer drugs have low response rates in a patient population. Biomarkers can be used to match patients to treatment strategies, for example, drugs that specifically target the molecular drivers of a given cancer. Immunohistochemistry is commonly used to measure expression of certain biomarkers. However specific antibodies are required for antigens of interest. This relationship limits the number of targets that can be evaluated and the amount of information that can be gleaned. DNA (e.g., exome) sequencing of tumor tissue has also been used to evaluate cancer samples. However, this method does not provide information about whether a gene is expressed, or if so, at what level.

RNA expression levels can provide a broader range of information than IHC or DNA sequencing can. Tumor RNA sequencing can reveal tumor antigens and targets expressed by cancer cells and provide information on the tumor microenvironment including immune response, the integrity of DNA repair pathways, and engagement of angiogenesis and other cancer-related pathways. RNA sequencing data can provide information that includes gene expression level, gene variants, mutations, epigenetic changes, e.g., gene silencing, and genomic rearrangements including gene amplifications and deletions.

### INCORPORATION BY REFERENCE

Each patent, publication, and non-patent literature cited in the application is hereby incorporated by reference in its entirety as if each was incorporated by reference individually.

### SUMMARY

Disclosed herein, in some aspects, is a method comprising: (a) processing gene expression counts of a test biological sample obtained from a test subject to obtain normalized gene expression values suitable for comparison to a database, wherein: the gene expression counts are generated by RNA sequencing of the test biological sample obtained from the test subject; the database comprises gene expression counts obtained from a plurality of control biological samples; and wherein each of the control biological samples is a sample type that is comparable to the test biological sample, and each of the control biological samples is independently obtained from a normal control subject; (b) identifying a gene that is aberrantly expressed in the test biological sample relative to the plurality of control biological samples; and (c) providing a wellness recommendation based on the gene that is aberrantly expressed in the test biological sample relative to the plurality of control biological samples.

Disclosed herein, in some aspects, is a method comprising processing gene expression counts of a test biological sample to obtain normalized gene expression values suitable for comparison to a database, wherein the database comprises gene expression counts from a plurality of control biological samples, wherein: (a) the gene expression counts of the test biological sample are: (i) generated by RNA sequencing of the test biological sample; (ii) subsampled to a target number of assigned reads; and (iii) sorted by a total of gene expression counts assigned to each gene, thereby generating sorted gene expression counts of the test biological sample; (b) the gene expression counts of each control biological sample of the plurality are: (i) generated by RNA sequencing of the control biological sample; (ii) subsampled to the target number of assigned reads; and (iii) sorted by a total of gene expression counts assigned to each gene, thereby generating sorted gene expression counts of the control biological sample; and (c) the processing comprises, for each position of the sorted gene expression counts of the test biological sample, calculating a normalized gene expression value from an average of: (i) gene expression count at the position of the sorted gene expression counts of the test biological sample; and (ii) gene expression count for each of the plurality of control biological samples at a corresponding position of the sorted gene expression counts of the control biological sample; thereby generating the normalized gene expression values suitable for comparison to the database.

Disclosed herein, in some aspects, is a computer program product comprising a non-transitory computer-readable medium having computer-executable code encoded therein, the computer-executable code adapted to be executed to implement a method, the method comprising: a) running a gene processing system, wherein the gene processing system comprises: i) an expression count processing component; ii) a gene identifying component; iii) a recommendation component; iv) a database of gene expression counts obtained from a plurality of control biological samples, wherein each of the control biological samples is a sample type that is comparable to a test biological sample, and each of the control biological samples is independently obtained from a normal control subject; and v) an output component; b) processing, by the expression count processing component, gene expression counts of RNA sequencing of the test biological sample obtained from a test subject to obtain gene expression values suitable for comparison to the database; c) identifying, by the gene identifying component, a gene that is aberrantly expressed in the test biological sample relative to the plurality of control biological samples; d) providing a wellness recommendation, by the recommendation component, based on the gene that is aberrantly expressed in the test biological sample relative to the plurality of control biological samples; and e) outputting, by the output component, a report that comprises the wellness recommendation.

Disclosed herein, in some aspects, is computer program product comprising a non-transitory computer-readable medium having computer-executable code encoded therein, the computer-executable code adapted to be executed to implement a method, the method comprising: a) running a gene processing system, wherein the gene processing system comprises: i) a database of gene expression counts obtained from a plurality of control biological samples; ii) a subsampling component; iii) a sorting component; iv) a normalizing component; and v) an output component; b) subsampling, by the subsampling component, gene expression counts of RNA sequencing of a test biological sample obtained from a test subject to a target number of assigned reads, thereby generating subsampled gene expression counts of the test biological sample; c) sorting, by the sorting component, a total of gene expression counts of the subsampled gene expression counts of the test biological sample to obtain sorted gene expression counts of the test biological sample; d) subsampling, by the subsampling component, gene expression counts of RNA sequencing of each control biological sample of the plurality to the target number of assigned reads, thereby generating subsampled gene expression counts of each of the control biological samples; e) sorting, by the sorting component, a total of gene expression counts of the subsampled gene expression counts of each of the control biological samples to obtain sorted gene expression counts of each of the control biological samples; f) normalizing, by the normalizing component, the sorted gene expression counts of the test biological sample to obtain normalized gene expression values of the test biological sample, wherein the normalizing comprises, for each position of the sorted gene expression counts of the test biological sample, calculating a normalized gene expression value from an average of: (i) gene expression count at the position of the sorted gene expression counts of the test biological sample; and (ii) gene expression count for each of the plurality of control biological samples at a corresponding position of the sorted gene expression counts of the control biological sample; and g) outputting, by the output component, the normalized gene expression values of the test biological sample.

### BRIEF DESCRIPTION OF THE FIGURES

FIG. 1 illustrates generation of a cDNA library from RNA.

FIG. 2 illustrates a sequencing strategy according to the present disclosure.

FIG. 3A illustrates subtraction of unique molecular identifiers (UMI) from reads.

FIG. 3B illustrates trimming of adapters on the 3′ end of a read and quality-trimming to facilitate better alignment to the reference genome.

FIG. 3C illustrates alignment of sequencing reads to the human reference genome.

FIG. 3D illustrates removal of PCR duplicates containing the same UMI.

FIG. 3E illustrates quantifying how many aligned sequencing reads were assigned to transcripts.

FIG. 4A illustrates high correlation of gene expression data from FFPE and FF samples according to methods of the disclosure.

FIG. 4B provides indicators of RNA quality (DV200, RQN) and Pearson correlation coefficients achieved by comparing RNA sequencing data generated using a method of the disclosure or a control method, from paired (i.e., same individual, same tumor) FFPE and FF sample sources.

FIG. 5A is a plot illustrating a classification scheme for gene expression disclosed herein.

FIG. 5B illustrates concordance of RNA expression data with IHC data. RNA expression data were processed by a method disclosed herein using as normal samples from normal subjects as control biological samples. TN, FP, FN, and TP represent number of true negatives, false positives, false negatives, and true positives, respectively. PPV and NPV are the positive predictive value and negative predictive value.

FIG. 5C illustrates concordance of RNA expression data with IHC data. RNA expression data were processed by a method disclosed herein using as normal adjacent tissues from the same subjects as the cancer samples as control biological samples. TN, FP, FN, and TP represent number of true negatives, false positives, false negatives, and true positives, respectively. PPV and NPV are the positive predictive value and negative predictive value.

FIG. 5D shows receiver operator characteristic (ROC) curves and the area under the curve (AUC) for ER, PR, and HER2 data generated by a method of the disclosure and compared to IHC data. Top panel: ER (ESR1), AUC=1; middle panel: PR (progesterone receptor/PGR), AUC=0.987; lower panel: HER2 (ERBB2), AUC=0.836.

FIG. 6 is a heatmap showing expression of CTA genes in breast cancer samples.

FIG. 7 illustrates expression of four cancer testis antigens in a triple negative breast cancer FFPE sample.

FIG. 8 illustrates very high or high expression of genes involved with immune checkpoints in a triple negative breast cancer FFPE sample, according to a classification scheme disclosed herein (for example, as illustrated in FIG. 5A).

FIG. 9 provides non-limiting examples of advantages of methods disclosed herein compared to DNA sequencing methods.

FIG. 10 demonstrates over-expression of several tumor antigens targeted by emerging immune therapies.

FIG. 11 illustrates design a hypothetical combinatorial study with 3 immune therapy targets and 1 checkpoint inhibitor (e.g. Pembrolizumab, anti-PDL1).

FIG. 12 depicts a log 2 RNA plot of EGFR expression in a breast cancer tissue sample as compared with control normal (left) and control tumor (right) ranges.

FIG. 13 depicts a log 2 plot of RNA expression levels of PARP1, PARP2, BRCA1, BRCA2, PTEN, ATM, RAD50, and RAD51C in a breast cancer tissue sample as compared with normal control ranges.

FIG. 14A depicts an illustrative plot showing thresholds for VERY LOW, LOW, HIGH, and VERY HIGH gene expression relative to normal tissue gene expression.

FIG. 14B illustrates normalized gene expression values of ER (ESR1) for samples of breast tissue processed according to the methods of the disclosure.

FIG. 14C illustrates normalized gene expression values of PR (PGR) for samples of breast tissue processed according to the methods of the disclosure.

FIG. 14D illustrates normalized gene expression values of HER2 (ERBB2) for samples of breast tissue processed according to the methods of the disclosure.

FIG. 15 is a heat map showing gene expression values generated from fresh frozen (FF) samples via a control method (left) compared to gene expression values generated from corresponding paired (i.e., same individual, same tumor) FFPE samples via a method disclosed herein (right). The x axis is for subjects, while each row is for a different gene identified as relevant to cancer therapeutics.

FIG. 16 summarizes a workflow of initial data processing to determine gene expression counts using as an input data from the Cancer Genome Atlas Breast Invasive Carcinoma (TCGA) and The Genotype-Tissue Expression (GTEx) databases.

FIG. 17A shows distribution of gene expression data for NRF1 from TCGA and GTEx sources prior to normalization. Samples are grouped by source—NAT: normal adjacent tissue from the TCGA dataset; NOR: normal control tissue from the GTEx dataset; TUMOR: primary tumor samples from the TCGA dataset.

FIG. 17B shows distribution of gene expression data for NRF1 from TCGA and GTEx sources after normalization. Samples are grouped by source—NAT: normal adjacent tissue from the TCGA dataset; NOR: normal control tissue from the GTEx dataset; TUMOR: primary tumor samples from the TCGA dataset.

FIG. 17C shows distribution of gene expression data for PUM1 from TCGA and GTEx sources prior to normalization. Samples are grouped by source—NAT: normal adjacent tissue from the TCGA dataset; NOR: normal control tissue from the GTEx dataset; TUMOR: primary tumor samples from the TCGA dataset.

FIG. 17D shows distribution of gene expression data for PUM1 from TCGA and GTEx sources after normalization. Samples are grouped by source—NAT: normal adjacent tissue from the TCGA dataset; NOR: normal control tissue from the GTEx dataset; TUMOR: primary tumor samples from the TCGA dataset.

FIG. 17E shows distribution of gene expression data for UBC from TCGA and GTEx sources prior to normalization. Samples are grouped by source—NAT: normal adjacent tissue from the TCGA dataset; NOR: normal control tissue from the GTEx dataset; TUMOR: primary tumor samples from the TCGA dataset.

FIG. 17F shows distribution of gene expression data for UBC from TCGA and GTEx sources after normalization. Samples are grouped by source—NAT: normal adjacent tissue from the TCGA dataset; NOR: normal control tissue from the GTEx dataset; TUMOR: primary tumor samples from the TCGA dataset.

FIG. 18A is a Precision-Recall plot of a training set to evaluate the ability of normalized gene expression values to discriminate between positive and negative status for ESR1/ER. The line near the bottom of the plot is the proportion of positive cases and represents a random classifier. The large, lighter dot represents the calculated ideal threshold using the maximum F-score.

FIG. 18B is a Precision-Recall plot of a training set to evaluate the ability of normalized gene expression values to discriminate between positive and negative status for PGR/PR. The line near the bottom of the plot is the proportion of positive cases and represents a random classifier. The large, lighter dot represents the calculated ideal threshold using the maximum F-score.

FIG. 18C is a Precision-Recall plot of a training set to evaluate the ability of normalized gene expression values to discriminate between positive and negative status for HER2. The line near the bottom of the plot is the proportion of positive cases and represents a random classifier. The large, lighter dot represents the calculated ideal threshold using the maximum F-score.

FIG. 19 shows the results of a PCA of unified RNA-seq datasets after normalization by a method disclosed herein.

FIG. 20 illustrates the proportion of tumors in which the indicated genes showed significant over-expression in NAT samples.

FIG. 21 illustrates the proportion of tumors in which the indicated genes showed significant under-expression in NAT samples.

FIG. 22 illustrates the proportion of tumor samples in which the indicated genes showed significant over-expression in NAT. The categories of drugs that target specific genes are labelled.

FIG. 23A shows normalized expression levels of druggable fusion genes in a metastatic thyroid cancer.

FIG. 23B provides therapeutics and clinical trials associated with genes detected in a metastatic thyroid cancer, and associated treatment recommendations.

FIG. 24 illustrates a computer system for facilitating methods, systems, products, or devices described herein.

FIG. 25A shows a heat map of correlation values for RNA samples after deduplication.

FIG. 25B shows a heat map of correlation values for RNA samples after deduplication and normalization by a method disclosed herein.

FIG. 25C shows a heat map of correlation values for RNA samples after deduplication and normalization by a Trimmed Measure of Means (control) method.

FIG. 25D shows a heat map of correlation values for RNA samples after deduplication and normalization by a Relative Log Expression (control) method.

FIG. 26A shows a heat map of correlation values for RNA samples after deduplication.

FIG. 26B shows a heat map of correlation values for fragmented RNA samples after deduplication and normalization by a method disclosed herein.

FIG. 26C shows a heat map of correlation values for fragmented RNA samples after deduplication and normalization by a Trimmed Measure of Means (control) method.

FIG. 26D shows a heat map of correlation values for fragmented RNA samples after deduplication and normalization by a Relative Log Expression (control) method.

FIG. 27A shows a heat map of correlation values for highly fragmented RNA samples after deduplication.

FIG. 27B shows a heat map of correlation values for highly fragmented RNA samples after deduplication and normalization by a method disclosed herein.

FIG. 27C shows a heat map of correlation values for highly fragmented RNA samples after deduplication and normalization by a Trimmed Measure of Means (control) method.

FIG. 27D shows a heat map of correlation values for highly fragmented RNA samples after deduplication and normalization by a Relative Log Expression (control) method.

### DETAILED DESCRIPTION

Patient responses to anti-cancer therapeutics vary widely. Tools to match patients to treatments are limited. Treatment decisions for cancer patients are often made based on limited data generated using traditional methods. For example, in the case of breast cancer, a tumor is largely characterized by ER, PR, and HER2 status based on techniques such as immunohistochemistry (IHC). However, cancer is a heterogeneous complex of diseases, and patients that have similar profiles for a few biomarkers may respond very differently to a given treatment regimen based on other factors, for example, mutations or expression levels of other oncogenes, tumor suppressor genes, immune checkpoint genes, etc. Methods that utilize a broader array of biomarkers for diagnostic purposes and for treatment decisions can produce better results.

RNA sequencing and other high throughput gene expression analysis methods have great potential for matching cancer patients to the newest targeted therapies, including cancer vaccines, immunotherapies, chemotherapies, and combinations thereof. RNA sequencing can provide data for vastly more potential targets and biomarkers than traditional methods, such as immunohistochemistry (IHC) or RT-qPCR. Furthermore, RNA sequencing can provide additional layers of data compared to DNA sequencing, allowing superior clinically actionable insights. For example, RNA sequencing provides expression data, and can delineate between alternatively spliced transcripts, and can have a superior sensitivity for detecting gene fusions.

However, RNA sequencing is under-utilized clinically due to complexity of data analysis and a lack of tools and techniques that link RNA sequencing data to clinical actions. A significant barrier to the use of RNA-sequencing in the clinic is a lack of methods and software to detect aberrant gene expression in tumor biopsies and other clinical samples from individual subjects. Software tools exist for identifying differential gene expression between two conditions. However, these tools generally require predefined groups of at least a certain size and/or require replicate samples, and limit the utility for clinical applications (e.g., where a single sample is obtained from a single patient). In some embodiments, a method disclosed herein allows accurate comparison of gene expression data from a single test biological sample to a plurality of control biological samples, and identification of aberrantly expressed gene(s) in the test biological sample based on the comparison.

The disclosure provides compositions and methods for quantifying the RNA transcription level of one or more genes in a test biological sample from a subject. Aberrantly expressed gene(s) can be identified and quantified, and the aberrantly expressed genes and/or their expression levels can be used to, for example, provide a wellness recommendation, design a therapeutic, diagnose a disease or condition, or a combination thereof. The wellness recommendation can be a treatment recommendation, which can include identifying a therapeutic that is likely to benefit the subject or not benefit the subject (e.g., a targeted therapy, cancer vaccine (e.g., mRNA vaccine), immunotherapy (e.g., checkpoint inhibitor, cell therapy), chemotherapy, clinical trials, or combination thereof).

Disclosed herein, in some embodiments, are methods of detecting, measuring, analyzing, and/or quantifying the RNA transcription level of one or more genes in a biological sample from a subject. Methods of the disclosure can be used, for example, to determine the presence or absence of a disease or condition, such as a cancer, or to identify a sub-type of the disease or condition, based on an altered RNA transcription level of the one or more genes.

The methods can include comparing a measured RNA transcription level of one or more genes (e.g., in a subject or a biological sample therefrom) to a control RNA transcription level. In some embodiments, the control RNA transcription level is from a control subject that does not have a cancer disclosed herein, for example, a healthy control or a normal control subject. The control RNA transcription level can be derived from a database of RNA transcription levels, for example, a database of RNA transcription levels associated with the absence of a disease or condition (e.g., associated with a healthy or normal control state). In some embodiments, the control RNA transcription level is from a second subject having a known disease or condition (for example, the same disease or condition or a different disease or condition to the first subject). The control RNA transcription level can be derived from a database of RNA transcription levels for the one or more genes correlated with a specific disease or condition. The control RNA transcription level can be from any suitable number of subjects, for example, a group of subjects as disclosed herein.

### Biological Sample

Methods disclosed herein can utilize one or more biological samples. For example, RNA can be extracted from a biological sample and subjected to RNA sequencing, and data obtained from the RNA sequencing can be processed to identify an aberrantly expressed gene, or for use as a control. A biological sample disclosed herein can be a test biological sample from a test subject, or a control biological sample from a control subject. Normalized gene expression values obtained from the test biological sample can be compared to normalized gene expression values from a plurality of control biological samples, for example, to identify one or more aberrantly expressed genes, as disclosed herein.

A biological sample can comprise or can be a liquid. A biological sample can be a liquid biopsy. In some embodiments, information (e.g., normalized gene expression values) obtained from a liquid biopsy can guide clinical treatment. For example, circulating Her2 RNAs can be used to monitor the response to Her2 therapies.

A biological sample can be or can comprise, for example, saliva, urine, blood (e.g., whole blood), plasma, serum, platelets, exosomes, cerebrospinal fluid, lymph, bodily fluid, tears, any other bodily fluid comprising RNA, or a combination thereof. A biological sample can be or can comprise, for example, a liquid tumor, such as cells of a hematologic cancer. A biological sample can comprise blood cells, for example, peripheral blood mononuclear cells (PBMCs). In some embodiments, a biological sample is saliva. In some embodiments, a biological sample is urine. In some embodiments, a biological sample is blood. In some embodiments, a biological sample is plasma. In some embodiments, a biological sample is serum. In some embodiments, a biological sample comprises breast tissue. In some embodiments, a biological sample comprises ovarian tissue, lung, bladder, colon, skin, prostate, liver, brain, pancreas, kidney, endometrial tissue, cervical tissue, bone, mouth, throat, thyroid, lymph node, blood, saliva, urine, or feces.

A biological sample can be or can comprise a solid. A biological sample can be or can comprise a solid tissue sample from any organ or tissue. A biological sample can be or can comprise a biopsy that comprises tumor tissue or is suspected to comprise tumor tissue.

A biological sample (e.g., a test biological sample or a control biological sample) can comprise tumor tissue, for example, of any cancer or tumor type disclosed herein. A biological sample (e.g., a test biological sample or a control biological sample) can comprise cancer cells, for example, of any cancer or tumor type disclosed herein.

A biological sample (e.g., a test biological sample or a control biological sample) can comprise predominantly cells from a specific organ or from a tissue within a specific organ. An organ can refer to a group of cells, for example, in a liquid or solid for, with or without an extracellular matrix. In some embodiments, cells within an organ (e.g., in a healthy subject) have a biological function that distinguishes them from other cells outside the organ. A biological sample can comprise or can be a tissue sample. A biological sample can be obtained as part of a biopsy. A biological sample can be obtained as part of a surgery.

A biological sample can comprise biological material that is fresh frozen (FF), fixed (e.g., in neutral buffered formalin or any other tissue fixative), formalin fixed paraffin embedded (FFPE), cryopreserved, incubated in RNA stabilizing reagents, or otherwise preserved or stabilized for the maximum recovery of RNA from within the sample. In some embodiments, the biological sample is treated in a manner that preserves the integrity of the RNA species until the RNA can be isolated from the sample, such as by freezing excised tissue in an RNA preserving solution such as RNALater from ThermoFisher Scientific (Waltham, MA) or Allprotect Tissue Reagent from Qiagen Sciences (Germantown MD). RNA that is partially degraded can still be analyzed. Subsequent steps in the process, e.g. sequence amplification, can be adjusted to work with fragmented and/or otherwise degraded RNA as disclosed herein. After isolation, additional precautions can be taken to protect the RNA sample from degradation, e.g., by RNAse enzymes. In some embodiments, a biological sample is an FFPE sample. In some embodiments, a biological sample is a fresh frozen sample. In some embodiments, a biological sample is a fresh sample.

A biological sample of the disclosure (e.g., a test biological sample or a control biological sample) can be from a subject. The subject can be an animal, e.g., a vertebrate. A biological sample can be from a subject that is a mammal. In some embodiments, the biological sample is from a subject that is a human. In some embodiments, the biological sample is from a subject that is a mouse, a rat, a cat, a dog, a rabbit, a cow, a horse, a goat, a monkey, a cynomolgus monkey, or a lamb. In some embodiments, the biological sample is from a subject that is a primate. In some embodiments, the biological sample is from a subject that is a non-human primate. In some embodiments, the biological sample is from a subject that is a non-rodent subject. A subject can be a female subject. A subject can be a male subject.

In some embodiments, a biological sample (e.g., a test biological sample or a control biological sample) is isolated from a subject that is being screened for cancer, is suspected of having cancer, is diagnosed with cancer, or is being monitored for cancer recurrence or relapse. The biological sample can comprise primary tumor tissue, metastatic tumor tissue, precancerous tissue, and/or tissue that is believed to contain tumor cells or precancerous cellular changes. The biological sample can contain tumor-infiltrating immune cells or other cells in the tumor tissue or in adjacent normal tissue. The biological sample can be a biological sample encountered in clinical pathology, including but not limited to, sections of tissues such as biopsy or tissue removed during surgical or other procedures, bodily fluids, autopsy samples, or frozen sections taken for histological purposes. Such biological samples can include blood and blood fractions or products, sputum, effusion, cheek cells tissue, patient-derived cultured cells (e.g., primary cultures, explants, and transformed cells), stool, urine, other biological or bodily fluids. etc.

A biological sample can be obtained from a subject before a treatment (e.g., administration of an anti-cancer therapeutic), during a treatment, or after a treatment. In some embodiments, biological samples are obtained from a subject before a treatment, during the treatment, and/or after the treatment.

A biological sample can be a test biological sample obtained from a test subject. The test subject can be a subject that has a disease or condition (e.g., a disease or condition disclosed herein, such as any type of cancer disclosed herein). The test subject can be a subject that is suspected of having a disease or condition. The test subject can be a subject that has or is suspected of having an acute disease. The test subject can be a subject that has or is suspected of having a chronic disease. The test subject can be a subject that has or is suspected of having an autoimmune disease. The test subject can be a subject that has or is suspected of having a metabolic disease. The test subject can be a subject that has or is suspected of having a neurological disease. The test subject can be a subject that has or is suspected of having a degenerative disease.

In some embodiments, the test subject does not have a disease or condition. In some embodiments, the test subject does not have or is not suspected of having a disease or condition. In some embodiments, it is unknown whether the test subject has a disease or condition.

In some embodiments, a method disclosed herein uses a single test biological sample obtained from a single test subject. In some embodiments, methods of the disclosure can be useful for identifying aberrantly expressed gene(s) from a single test biological sample obtained from a single test subject, for example, with superior accuracy compared to alternative methods. In some embodiments, two or more test biological samples are obtained from a single test subject. In some embodiments, test biological samples are obtained from two or more test subjects (e.g., a plurality of test subjects, such as one test biological sample per subject, or two or more test biological samples from a test subject). In some embodiments, a single test biological sample is obtained from each of a plurality of test subjects. In some embodiments, two or more test biological samples are obtained from each of a plurality of test subjects.

In some embodiments, an initial test biological sample is obtained from a test subject and a subsequent test biological sample is obtained from the test subject later (e.g., months or years later). A first wellness recommendation can be provided based on the initial test biological sample and a second wellness recommendation can be provided based on the subsequent test biological sample.

A test biological sample can be or can comprise a sample that is healthy or normal. A test biological sample can be or can comprise a sample from a tissue that is healthy or normal. A tissue that is healthy or normal can lack a specific pathological diagnosis (e.g., disease diagnosis). For example, the tissue that is healthy or normal can lack a cancer diagnosis. In some embodiments, a tissue that is healthy or normal lacks a specific pathological diagnosis, but comprises a different pathological diagnosis.

In some embodiments, a test biological sample is or has been examined by a certified clinical pathologist. In some embodiments, the test biological sample is subjected to laboratory diagnostic tests (such as immunohistochemical assays or array CGH) to confirm that the biological sample is diseased or non-diseased and is of the assumed sample type (e.g., the tissue, biological fluid, cell type, cell line, cancer type etc.).

A biological sample can be a control biological sample obtained from a control subject. The control subject can be, for example, a normal subject that does not have a given cancer.

A control biological sample can be or can comprise a sample that is healthy or normal. A control biological sample can be or can comprise a sample from a tissue that is healthy or normal. A tissue that is healthy or normal can lack a specific pathological diagnosis (e.g., disease diagnosis). For example, the tissue that is healthy or normal can lack a cancer diagnosis. In some embodiments, a tissue that is healthy or normal lacks a specific pathological diagnosis, but comprises a different pathological diagnosis. For example, a control biological sample that is a bone sample can be a biological sample from a bone that does not contain signs of bone cancer or metastasis can contain signs of a separate pathological process, for example, osteoarthritis or loss of bone density. The control biological sample that is a bone sample can be a biological sample from a bone that is negative for or not diagnosed as having a bone cancer or cancer metastasis, but that is positive for or has been diagnosed as having a separate pathological process, for example, osteoarthritis or loss of bone density. In some embodiments, a tissue that is healthy or normal can lack any pathological disease diagnosis. A control biological sample can be a non-diseased biological sample. A control biological sample can be obtained clinically, from a collaborator, purchased from a commercial biorepository, or otherwise procured.

A control biological sample can be obtained from a control subject. A control biological sample can be or can comprise a sample (e.g., tissue sample) from a control subject. A control subject can be a normal subject. A control subject can be a healthy subject. A control subject can be a subject that has not been diagnosed with cancer. A control subject can be a subject that has not been diagnosed with a specific disease or condition, for example, a disease or condition that a test subject has or is suspected of having. In some embodiments, a control subject does not have a specific disease or condition, but the subject does have a different disease or condition (e.g., does the control subject does not have cancer, but does have type 2 diabetes). A control subject can be a subject that is not suspected of having a disease or condition that a test subject has or is suspected of having. In some embodiments, a control subject does not have any diagnosed disease. In some embodiments, a control subject does not have any diagnosed chronic disease. In some embodiments, a control subject does not have any diagnosed cancer. In some embodiments, a control subject does not have or has not been diagnosed with a type of cancer disclosed herein.

In some embodiments, a control subject has a disease or condition. In some embodiments, a control subject has a disease or condition that is the same as a disease or condition that a test subject has or is suspected of having. In some embodiments, a control subject has a disease or condition that is different than a disease or condition that a test subject has or is suspected of having.

In some embodiments, a control biological sample (e.g., that is used to calculate a normal reference range) is or has been examined by a certified clinical pathologist. In some embodiments, the control biological sample is subjected to laboratory diagnostic tests (such as immunohistochemical assays or array CGH) to confirm that the biological sample is diseased or non-diseased and is of the assumed sample type (e.g., the tissue, biological fluid, cell type, cell line, etc.) In some embodiments, the RNA transcription level of a control biological sample is compared to existing RNA transcription levels of known non-diseased biological samples.

A control biological sample can be from a comparable tissue type as a test biological sample. A comparable tissue type to a tissue type of interest can comprise a shared or similar function as the tissue type of interest. A comparable tissue type to a tissue type of interest can comprise a same cell type as the tissue type of interest. A comparable tissue type to a tissue type of interest can comprise a same predominant type as the tissue type of interest. A comparable tissue type to a tissue type of interest can comprise similar ratio of cell types as the tissue type of interest. In some embodiments, at least 20%, at least 30%, at least 40%, at least 50%, at least 50%, at least 60% at least 70%, at least 80%, or at least 90% of cells in the comparable tissue type are the same cell type as cells in the tissue type of interest.

A control biological sample can be from a same tissue type as a test biological sample. A control biological sample can be from a tissue type that is substantially the same as a tissue type of a test biological sample. In some embodiments, a control biological sample is from a different tissue type than a test biological sample.

A control biological sample can be a comparable sample type as a test biological sample. A control biological sample can be a comparable sample type as a test biological sample. A control biological sample can be of a sample type that is substantially the same as a sample type of a test biological sample. In some embodiments, a control biological sample is a different sample type than a test biological sample.

In some embodiments, a test subject has a cancer that has metastasized to a metastatic site, and a control biological sample is of a comparable tissue type as a tissue type in the metastatic site. In some embodiments, test subject has a cancer that has metastasized to a metastatic site, and a control biological sample is of a same tissue type as a tissue type in the metastatic site. In some embodiments, test subject has a cancer that has metastasized to a metastatic site, and a control biological sample is substantially similar or substantially same sample type as a tissue type in the metastatic site. In some embodiments, test subject has a cancer that has metastasized to a metastatic site, and a control biological sample is substantially similar or substantially same tissue type as a tissue type in the metastatic site.

A test subject can be matched to a control subject or a plurality thereof, for example, based on age, sex, ethnicity, disease risk factors, diagnosis, clinical or pathological characteristics of a disease, other factors, treatment history, or a combination thereof.

Methods disclosed herein can utilize a plurality of control biological samples. A database can comprise gene expression data (e.g., gene expression counts or normalized gene expression values) from a plurality of control biological samples as disclosed herein.

A plurality of control biological samples can comprise, for example, at least 2, at least 3, at least 4, at least 5, at least 6, at least 7, at least 8, at least 9, at least 10, at least 15, at least 20, at least 25, at least 40, at least 50, at least 75, at least 100, at least 200, at least 300, at least 400, at least 500, at least 1,000, or at least 10,000 control biological samples.

A plurality of control biological samples can comprise or contain, for example, at most 5, at most 10, at most 15, at most 20, at most 25, at most 40, at most 50, at most 75, at most 100, at most 200, at most 300, at most 400, at most 500, at most 1,000, at most 10,000, or at most 100,000 control biological samples.

A plurality of control biological samples can comprise, for example, about 2, about 3, about 4, about 5, about 6, about 7, about 8, about 9, about 10, about 15, about 20, about 25, about 40, about 50, about 75, about 100, about 200, about 300, about 400, about 500, about 1,000, or about 10,000 control biological samples.

Each of the control biological samples can be independently obtained from a subject. Each of the control biological samples can be independently obtained from a normal control subject. Each of the control biological samples can be independently obtained from a healthy control subject.

A test biological sample and each of a plurality of control biological samples can be a comparable sample type (e.g., comparable tissue type). A test biological sample and each of a plurality of control biological samples can be a same sample type (e.g., same tissue type). A test biological sample and each of a plurality of control biological samples can be a substantially similar sample type (e.g., substantially similar tissue type). A test biological sample and each of a plurality of control biological samples can of a sample type (e.g., tissue type) that are substantially the same.

In some embodiments, a method of the disclosure does not utilize a control biological sample that is obtained from the test subject, for example, does not utilize an adjacent normal or matched normal sample obtained from the test subject. Methods disclosed herein can comprise using control biological samples that are not adjacent normal samples, for example, that are not obtained from a morphologically or histologically normal part of a tissue adjacent to a test biological sample (e.g., comprising cancer tissue) of a test subject. In some embodiments, an adjacent normal tissue can comprise a modified gene expression signature compared to an average gene expression signature of true normal control biological samples obtained from subjects that do not have a disease or condition the test subject has, e.g., cancer.

Methods disclosed herein can comprise using control biological samples that are not matched normal samples from a test subject, for example, that are not obtained from a morphologically or histologically normal tissue from a same subject as a test biological sample. A matched normal can be, for example, a blood sample, peripheral blood mononuclear cells, an adjacent normal tissue, a corresponding normal tissue (e.g., from a contralateral side compared to a test biological sample, such as a sample of a healthy left lung when a test biological sample is a sample of a diseased right lung). In some embodiments, a matched normal tissue from a test subject can comprise a modified gene expression signature compared to an average gene expression signature of true normal control biological samples obtained from subjects that do not have a disease or condition the test subject has, e.g., cancer.

In some embodiments, a control biological sample is derived from the test subject and is tumor-adjacent. In some embodiments, a control biological sample is not derived from the same test. In some embodiments, the control biological sample is not tumor-adjacent tissue from the same subject.

Gene expression reference profiles can be generated by analyzing RNA from control biological samples.

In some embodiments, the normal reference is an average of true normal tissue expression levels in control biological samples from normal or healthy individuals, while the test biological sample is from the corresponding organ or tissue type of a subject suffering from a condition. The disease or condition can be associated with or result in, for example, aberrant gene expression compared to an average of true normal tissue expression levels in the control biological samples from the normal or healthy individuals.

The RNA transcription level of a given gene in a test biological sample can be compared to a reference range for a control RNA transcription level in a relevant control subject population, e.g., a diseased population or a normal population. Control biological samples can be selected and grouped into different reference cohorts based on information provided in clinical pathology reports. For example, the RNA transcription level of progesterone receptor from a suspected breast cancer test biological sample can be compared with a first reference range for a control RNA transcription level of progesterone receptor in normal breast tissue, and can also be compared to a second reference range of triple negative breast cancer tissue, and a third reference range for estrogen receptor positive, HER2 negative breast cancer tissue. The diagnosis and subtype of diseased control biological samples can be confirmed by other laboratory analyses and/or by evaluation by a certified clinical pathologist. Diseased control biological samples can be selected and grouped into reference cohorts based on responders and non-responders to specific therapies.

In some embodiments, the RNA transcription levels in the test biological sample and the control biological sample are measured using the same RNA sequencing method and/or bioinformatics pipeline. In some embodiments, methods of the disclosure allow the RNA transcription levels in the test biological sample and the biological sample to be compared despite use of using different RNA sequencing methods and/or partially different bioinformatics processing pipelines, for example, due to a method of normalization disclosed herein.

In some embodiments, methods of the disclosure allow gene expression counts from control biological samples to be obtained from suitable sources, for example, databases, such as a gene expression atlas or repository. Suitable sources can include repositories of gene expression data that are not suitable to use as controls for many alternative methods. Thus, in some embodiments, methods of the disclosure allow clinical data sources to be harnessed in new and powerful ways. For example, data generated by the TCGA Research Network (cancergenome.nih.gov) includes gene expression counts derived from both microarrays and RNA sequencing for numerous tumors from different cancer types. In some embodiments, RNA sequencing data can be used to compute reference ranges or to obtain a distribution of control normalized gene expression values for a method disclosed herein. In some embodiments, microarray data can be used to compute reference ranges or to obtain a distribution of control normalized gene expression values for a method disclosed herein.

Data generated by the TCGA Research Network can be obtained from the National Cancer Institute's Genomic Data Commons Portal (gdc.cancer.gov/) and the Broad Institute's GDAC Firehose (gdac.broadinstitute.org/). Additional global gene expression data sets can be obtained from the websites of NCBI GEO (Gene Expression Omnibus at www.ncbi.nlm.nih.gov/geo), ENA (European Nucleotide Archive at www.ebi.ac.uk/ena), the GTEx Portal (www.gtexportal.org), and other online data repositories.

### RNA Sequencing

Methods disclosed herein can utilize RNA sequencing or data (e.g., gene expression counts) that have been generated by RNA sequencing. RNA sequencing can include any one or more of, for example, RNA isolation, laboratory processing of samples comprising RNA (e.g., including de-crosslinking, DNase treatment, purification, concentration, etc.), fragment analysis, poly(T) priming, random priming, reverse transcription, indexing (e.g., with universal molecular identifier (UMI) and/or universal dual index (UDI) sequences), library preparation, library amplification, sequencing, initial processing of raw sequencing data to generate gene expression counts, other elements disclosed herein, and combinations thereof.

RNA, such as messenger RNA (mRNA), can be isolated from biological samples (e.g., test or control biological samples) using any suitable extraction methods and reagents. In some embodiments, the RNA comprises, consists essentially of, or consists of mRNA. In some embodiments, the RNA is enriched for mRNA. In some embodiments, the RNA is depleted for rRNA and/or globulin RNA (e.g., using a GLOBINclear™ kit for globin mRNA depletion).

In some embodiments, RNA isolation can be performed using reagent kits and protocols from commercial manufacturers. For example, total RNA from breast tissue can be isolated using RNeasy lipid tissue kit from Qiagen. Additional examples of kits for RNA extraction include those made by Qiagen and ThermoFisher. The RNA isolation reagents and method used can be tailored to the biological sample type to improve the yield and quality of the RNA molecules that are retrieved from the biological sample, e.g., as disclosed herein. If a kit for extraction of total RNA is used, then the mRNA component of the total RNA can be subsequently isolated from the total RNA using any of several methods, for example, by capture on by poly(dT) magnetic beads.

Common tissue processing practices for clinical samples can present a challenge for obtaining usable RNA sequencing data. For example, clinical samples are commonly formalin fixed and paraffin embedded (FFPE) to allow cutting of sections, mounting on slides, and staining with various reagents to facilitate histopathological evaluation. RNA can be extracted from such FFPE samples but the extract is generally low quality, highly fragmented, and difficult to analyze compared to RNA obtained from fresh or fresh frozen tissue.

In some embodiments, methods of the disclosure provide improvements in wet lab and/or bioinformatics methods for generating high quality data from degraded RNA. If a sample is suspected of containing degraded RNA, e.g., the tissue has been preserved by formalin fixation and paraffin embedding (FFPE), then an isolation method tailored to degraded RNA (e.g., FFPE) samples can be used.

In some embodiments, a method disclosed herein for generating higher quality data from degraded RNA comprises de-crosslinking, for example, for a longer duration than alternative methods. In some embodiments, a method disclosed herein for generating higher quality data from degraded RNA comprises de-crosslinking by incubating at about 80° C. for at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, or at least about 30 minutes. In some embodiments, a method disclosed herein for generating higher quality data from degraded RNA comprises de-crosslinking by incubating at about 80° C. for about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 minutes. The de-crosslinking incubation can be one incubation or can be split between two incubations. The de-crosslinking incubation can be prior to proteinase K treatment (e.g., at 60° C.), after proteinase K treatment, or a combination thereof. For example, in some embodiments, the de-crosslinking comprises ten minutes of de-crosslinking incubation at 80° C. (e.g., in two five minute incubations) prior to proteinase K treatment, then an additional 15 minute de-crosslinking incubation at 80° C. after proteinase K treatment.

In some embodiments, a method disclosed herein for generating higher quality data from degraded RNA comprises a DNAse treatment, for example, two DNase treatments, followed by purification and/or concentration of RNA.

A degree of RNA degradation can be calculated as a DV200 value, wherein DV200=[fragments >200 bases/(fragments >200 bases+fragments <200 bases)].

In some embodiments, the disclosure provides improvements in wet lab and/or bioinformatics methods that facilitate generation of high quality RNA sequencing data that can be used in methods disclosed herein for RNA (e.g., from an FFPE biological sample) with a DV200 value of less than about 5%, less than about 10%, less than about 15%, less than about 20%, less than about 25%, less than about 30%, less than about 35%, less than about 40%, less than about 45%, or less than about 50%.

In some embodiments, a DV200 value of an RNA sample utilized in a method of the disclosure is at least about 1%, at least about 2%, at least about 3%, at least about 4%, at least about 5%, at least about 6%, at least about 7%, at least about 8%, at least about 9%, at least about 10%, at least about 5%, at least about 10%, at least about 15%, at least about 20%, at least about 25%, at least about 30%, at least about 35%, at least about 40%, at least about 45%, or at least about 50%.

Once isolated, RNA can be diluted in RNase free water or a suitable buffer prior to further analysis. RNA can be temporarily stored between steps at reduced temperature to prevent further degradation. The isolated RNA can further be evaluated for quality and yield using capillary electrophoresis with fluorescence detection using suitable kits and instruments, such as the Fragment Analyzer from Advanced Analytical (Alkeny, Iowa) or TapeStation from Agilent (Santa Clara, CA).

Quantification of RNA transcription level can be performed by any suitable methods including those described herein. When using sequencing for the quantification of RNA expression, gene expression counts can be generated by counting statistics of RNA sequencing data obtained from a test biological sample. Sequencing the RNA can occur from the 3′-end, the 5′-end, or non-discriminately, e.g., full length. In some embodiments, the method of quantifying an RNA transcription level of a gene in a biological sample involves (a) extracting RNA from a biological sample from the subject, and (b) measuring the RNA using an RNA sequencing method or kit comprising: (1) sequencing the RNA from the 3′-end, and (2) identifying the RNA, thereby quantifying the RNA transcription level of the gene.

In some embodiments, methods of the disclosure comprise sequencing RNA. RNA sequencing can comprise sequencing in a direction that corresponds to from the 5′-end of the original mRNA, from the 3′-end of the original mRNA, or from both ends. In some embodiments, the method comprises identifying the RNA.

In some embodiments, the RNA, e.g., the mRNA component of the RNA, is sequenced using a suitable quantitative RNA sequencing method. RNA sequencing can be performed through the use of a next generation sequencing (NGS) technology, e.g., massively parallel sequencing technology that produces many hundreds of thousands or millions of reads, e.g., simultaneously. Next generation sequencing platforms and reagent kits are available from, for example, Illumina, ThermoFisher Scientific, Pacific Biosciences, Oxford Nanopore Technologies, and Complete Genomics.

Quantitative RNA sequencing data analysis methods can be performed by using a software program executed by a suitable processor. The program can be embodied in software stored on a tangible medium such as CD-ROM, a hard drive, a DVD, or a memory associated with the processor, or the entire program or parts thereof could alternatively be executed by a device other than a processor, and/or embodied in firmware and/or dedicated hardware.

In some embodiments, quantitative RNA sequencing methods that are suitable for global transcript and gene expression analysis can generally be divided into two groups: tag-based methods that sequence a short segment or tag from each mRNA molecule analyzed, and full transcript methods that sequence the majority of bases from each mRNA molecule analyzed.

Representative tag-based methods for sequencing-based gene expression analysis include but are not limited to Serial Analysis of Gene Expression (SAGE) gene expression analysis by massively parallel signature sequencing (MPSS), and 3′ mRNA sequencing methods, such as Tag-seq, QuantSeq, TruQuant, and 3Seq. 3′ mRNA sequencing methods often do not require the use of restriction enzymes, and commercial reagent kits are available. For example, QuantSeq, MACE-Seq, and TruQuant kits.

In some embodiments, RNA sequencing comprises a reverse transcriptase enzyme. In some embodiments, the reverse transcriptase enzyme does not have a GC bias. MonsterScript™ Reverse Transcriptase from Illumina is an example of a reverse transcriptase enzyme. Other non-limiting examples of reverse transcriptase enzymes include the SuperScript reverse transcriptase enzymes from ThermoFisher Scientific, e.g., SuperScript II, SuperScript III, SuperScript IV, and SuperScript VILO mix.

Methods disclosed herein can comprise adjustment for PCR bias. Adjustment for PCT bias can comprise, for example, the use of unique molecular identifiers (UMIs). In some embodiments, methods of the disclosure comprise a unique molecular identifier (UMI). Non-limiting examples of UMI include xGen unique dual index UMI adapters (Integrated DNA Technologies) and Unique Molecular Identifier (UMI) Second Strand Synthesis Module for QuantiSeq FW. Adjustment for PCR bias can be done, to remove or reduce duplicate reads, for example, unique molecular identifiers can be used to remove duplicate reads during data processing.

Methods disclosed herein can utilize Unique Molecular Identifiers (UMIs). For example, a UMI can be appended to each RNA molecule, and the UMIs can be used to deduplicate reads during data processing.

Methods disclosed herein can comprise dual indexing (e.g., unique dual indexing). Dual indexes can be used, for example, to tag sequences originating from a common sample to facilitate demultiplexing of sequencing data (e.g., generated from multiple biological samples). Unique dual indexing can be used to filter index-hopped reads seen in downstream analyses. Misassigned reads can be flagged as undetermined reads and can be excluded from analysis.

Adjustment for PCR bias can be done, e.g., when sample sizes are small and/or when more PCR cycles are needed during amplification.

Additional types of RNA sequencing methods include non-digital methods. Non-digital RNA sequencing methods can involve enriching RNA for mRNA by poly(A) selection and/or depletion of rRNA, converting mRNA into cDNA using a reverse transcriptase reaction, ligating to sequencing adapters and transcript-specific and/or sample-specific identifier sequences (e.g., barcodes, such as unique molecular identifiers (UMIs) and unique dual indexes (UDIs)), amplifying the resulting constructs, and then sequencing. The mRNA can be optionally fragmented prior to the reverse transcription step, and the cDNA can be optionally fragmented post reverse transcription. An index DNA code (e.g., index) can be ligated prior to an amplification step, allowing multiplex amplification of several samples prior to the sequencing. The index can also be included on one of the PCR primers.

One variable in sequencing measurements is read depth, which can describe the total number of sequence reads analyzed from the sample. A sufficient read depth can be necessary to detect clinically relevant genes that are weakly expressed in biological (e.g., tumor) samples. For example, PD-1 and PD-L1 genes can be weakly expressed in solid tumors. In some embodiments, a minimum of 50 million reads, such as 100 million reads, can provide sufficient read depth for non-targeted full transcript sequencing. In some embodiments, methods of the disclosure comprise sequencing to a depth of at least 2 million, at least 4 million, at least 6 million, at least 8 million, at least 10 million, at least 15 million, at least 20 million, at least 30 million, at least 40 million, at least 50 million, at least 75 million, at least 100 million, at least 200 million, at least 300 million, at least 400 million, or at least 500 million reads.

Compared to alternative methods, tag-based sequencing methods, including 3′ mRNA sequencing, can require fewer reads, e.g., from five to ten times fewer, to detect the same clinically relevant genes. For targeted sequencing approaches, the total number of sequencing reads required to detect each target gene can depend on the composition of the assay panel.

RNA sequencing can generate reads of any type of RNA. In some embodiments, RNA sequencing generates reads of mRNAs. In some embodiments, RNA sequencing generates reads of non-coding RNAs. In some embodiments, RNA sequencing generates reads of coding RNAs. In some embodiments, RNA sequencing generates reads of micro RNAs.

### Initial Processing of RNA Sequencing Data

The output of an RNA sequencing assay can be summarized in a gene expression count table containing a group (e.g., list) of genes and associated gene expression counts, which can be a number (or estimated number) of detected RNA transcripts assigned to each gene. Such a gene expression count table can be a representation of the gene expression profile in a sample.

In some embodiments, a gene expression count table is generated from raw sequencing data. Gene expression counting can be performed by using one or more software programs executed by a suitable processor. Suitable software and processors can be commercially or publicly available software and processors or other software and processors disclosed herein. An illustrative example of generation of a gene expression count table from raw sequencing data is provided in EXAMPLE 2. Non-limiting examples of software programs, tools, and interfaces that can be used in methods of the disclosure include any suitable versions of BCL2FASTQ, BaseSpace Command Line Interface, SevenBridges Python API, AWS command line interface, FASTQC, UMI-tools, BBduk, STAR, SAMtools, HTSeq-count, Picard, and the like.

In some embodiments, a gene expression count table is obtained from a database.

RNA sequencing in this disclosure can comprise initial processing of RNA sequencing data. Initial processing of RNA sequencing data can comprise all the steps and programs necessary to calculate gene expression counts (e.g., a gene expression count table comprising the gene expression counts). Initial processing of RNA sequencing data can comprise, for example, conversion of raw data files to FASTQ files, quality control evaluation of reads, deduplication, adapter sequence trimming, quality trimming, alignment, alignment sorting and indexing, and transcript quantification, or any combination thereof.

Initial processing of RNA sequencing data can comprise, for example, conversion of raw data files (e.g., binary base call (BCL) format files) to FASTQ format files. Any suitable program can be used for conversion of raw data files to FASTQ format files, including but not limited to BCL2FASTQ.

Initial processing of RNA sequencing data can comprise, for example, quality control evaluation of reads (e.g., FASTQ reads). Any suitable program can be used for quality control evaluation of reads, including but not limited to FASTQC.

Initial processing of RNA sequencing data can comprise, for example, deduplication to reduce errors from duplicate reads (e.g., that were introduced from PCR). Any suitable program or tool can be used for deduplication, including but not limited to UMI-tools or Picard.

Initial processing of RNA sequencing data can comprise, for example, adapter sequence trimming. Adapter sequence trimming can increase alignment quality by removing adapter sequences introduced through the library preparation steps. Any suitable program can be used for adapter sequence trimming, including but not limited to BBduk.

Initial processing of RNA sequencing data can comprise, for example, quality trimming. Quality trimming can increase alignment quality by removing low quality parts of reads, e.g., from the 5′ and/or 3′ end. Any suitable program can be used for quality trimming, including but not limited to BBduk.

Initial processing of RNA sequencing data can comprise, for example, alignment, e.g., to a reference genome (e.g., a human reference genome, such as Genome Reference Consortium Human Build version 38 Human Genome (GRCh38) or an updated version thereof). Any suitable program can be used for alignment, including but not limited to STAR.

Initial processing of RNA sequencing data can comprise, for example, alignment sorting and indexing. Any suitable program can be used for alignment sorting and indexing, including but not limited to SAMtools.

Initial processing of RNA sequencing data can comprise, for example, transcript quantification (e.g., to generate gene expression counts that quantify how many aligned sequencing reads are assigned to each gene/transcript). Any suitable program can be used for transcript quantification, including but not limited to HTSeq-count.

Processing (e.g., initial processing) of RNA sequencing data can involve applying quality filters to reject sequence reads or parts thereof suspected of containing errors (for example, errors from the sequencing or from the library preparation), removing, e.g. trimming, adapter sequences, correcting for amplification bias, mapping the sequenced reads to a database of human genome and/or transcriptome sequences (e.g., the human RefSeq database), or any combination thereof. Sequence reads that map to the same gene can be combined to produce the gene expression count table.

In some embodiments, the sequence reads mapping to each RNA transcript are individually combined to generate a transcript count table. The gene expression count data can be given as raw sequencing reads, scaled to the total number of reads as disclosed herein (e.g., as transcripts per million reads) or as estimated reads.

Tag-based sequencing methods can produce a single sequencing read from each transcript. In some embodiments, the gene expression count data obtained from such tag-based sequencing methods can be processed without correcting for variations in gene length. In some embodiments, for full transcript sequencing approaches, the gene expression count data can be corrected for variations in transcript length, e.g., longer transcripts can generate more fragments and thus more reads per gene, and coverage.

In some embodiments, gene expression counts disclosed herein comprise global gene expression count data (e.g., for all genes). Gene expression count tables generated from global gene expression measurements can include expression data for >17,000 genes (e.g., about or more than 20,000 genes). The maximum number of genes included in the count table can depend upon what genes can be identified through the combination of the mapping and reference sequence database.

In some embodiments, a subset of genes is selected for inclusion in the gene expression count table. For example, a set of genes known to be clinically significant in a cancer, such as a type of cancer disclosed herein, can be selected for inclusion in a gene expression count table. The set of genes can be, for example, a set of genes that are clinically significant in breast cancer, such as triple-negative breast cancer. In some embodiments, a subset of genes that are associated with responsiveness of cancer to a treatment is selected for inclusion in the gene expression count table. In some embodiments, a subset of genes selected for inclusion in the gene expression count table comprise a set of genes contained in a database disclosed herein.

In some embodiments, a subset of genes that are associated with cancer responsiveness to an immune checkpoint inhibitor is selected for inclusion in the gene expression count table. In some embodiments, a subset of genes that are associated with cancer responsiveness to an immunotherapy is selected for inclusion in the gene expression count table. In some embodiments, a subset of genes that are associated with cancer responsiveness to a biologic is selected for inclusion in the gene expression count table. In some embodiments, a subset of genes that are associated with cancer responsiveness to a drug is selected for inclusion in the gene expression count table. In some embodiments, a subset of genes that are associated with cancer responsiveness to a chemotherapy is selected for inclusion in the gene expression count table. In some embodiments, a subset of genes that are associated with cancer responsiveness to a cell therapy is selected for inclusion in the gene expression count table.

In some embodiments, a subset of genes that are associated with cancer responsiveness to a treatment being evaluated in a clinical trial is selected for inclusion in the gene expression count table.

In some embodiments, a subset of genes that are associated with cancer responsiveness to a cancer vaccine is selected for inclusion in the gene expression count table. In some embodiments, a subset of genes that are suitable for inclusion in a cancer vaccine is selected for inclusion in the gene expression count table. In some embodiments, a subset of genes that are included in a cancer vaccine (e.g., antigens therefrom or mRNAs encoding the same) is selected for inclusion in the gene expression count table.

If a strand specific RNA sequencing method is used, the gene expression count table can optionally include read counts for antisense genes.

The gene expression count table can also contain further information for each gene such as, but not limited to, the full name of the gene, alternative gene symbol(s), the chromosomal location of the gene, or a list of the names of individual transcripts to which reads assigned to that gene were mapped. Gene expression count tables can be stored as text files or other formats and imported into commercial or proprietary data analysis software for inspection and analysis.

Targeted sequencing and other quantitative RNA analysis methods can produce gene expression count tables for genes included in an assay. Targeted assay panels can measure from 10 to over 1,000, e.g., about 50, about 100, about 150, about 200, about 300, about 400, or about 500 genes or more. In some embodiments, greater than 1,000 genes are measured in a targeted assay panel.

### Normalized Gene Expression Values

Methods of the disclosure can comprise generating and/or utilizing normalized gene expression values. To compare an RNA transcription level to a control RNA transcription level, measurements of gene expression (e.g., gene expression counts) can be and placed on a common scale (i.e., normalized to generate normalized gene expression values) such that quantitative comparisons can be made between, for example, samples, subjects, testing batches, operators, and testing sites, e.g., for which quantitative comparisons cannot otherwise be performed. Normalization by methods disclosed herein can allow comparison (e.g., quantitative comparison) of normalized gene expression values of a test biological sample (e.g., a single test biological sample) to normalized gene expression values of a plurality of control biological samples, which can facilitate identification of a gene that is aberrantly expressed in the test biological sample relative to the plurality of control biological samples.

Normalization or calculation of normalized gene expression values as disclosed herein can facilitate more accurate identification of aberrantly expressed genes in a clinically-useful context, for example, from a single clinical sample without requiring cohorts and replicates. Normalization or calculation of normalized gene expression values as disclosed herein can reduce or remove bias based on sample source, allowing, for example, comparison of samples from different sources, or use of databases as controls for identifying aberrant gene expression.

In some embodiments, RNA sequencing and/or initial processing of RNA sequencing data to generate gene expression counts are done in a reproducible manner.

Normalization of quantitative RNA sequencing data and other gene expression data can be required to detect differences in gene expression between a test biological sample and corresponding control biological samples, e.g., for identification of one or more aberrantly expressed gene(s) in the test biological sample relative to corresponding normal, healthy and/or diseased controls. Normalization strategies can be necessary to correct for sample-to-sample distributional differences in total gene expression counts, and/or within-sample gene-specific effects, such as gene length or GC-content effects.

The normalization can be performed by computer software. The normalization can be performed by a computer program product comprising a non-transitory computer-readable medium having computer-executable code encoded therein.

In some embodiments, gene expression count data of a test biological sample is normalized alongside or together with gene expression profiles derived from a set of reference samples, e.g., one or more, 2 or more, 3 or more, 4 or more, 5 or more, 8 or more, 10 or more, 20 or more, 30 or more, 40 or more, 50 or more, 100 or more, 200 or more, 500 or more, or 1,000 or more reference samples.

Normalized gene expression values of a test biological sample and a plurality of control biological samples can be normalized using a common (e.g., same) normalization technique.

In some embodiments, gene expression count data of a test biological sample is normalized alongside or together with other gene expression count data sets derived from one or more, e.g., 2 or more, 3 or more, 4 or more, 5 or more, 8 or more, 10 or more, 20 or more, 30 or more, 40 or more, 50 or more, 100 or more, 200 or more, 500 or more, or 1,000 or more control biological samples as disclosed herein (e.g., tissue samples from comparable tissue types of normal or healthy controls that lack a cancer).

In some embodiments, gene expression count data of a test biological sample is normalized separately to gene expression count data from control biological samples. For example, normalized gene expression values can be obtained from a first data set that comprises the control biological samples, and normalized gene expression values can be independently obtained from a second data set comprising gene expression values from the test biological sample(s). The independently normalized gene expression values of the test biological sample can be suitable for comparison to the normalized gene expression values from the control biological samples, e.g., to reference ranges therefrom and/or for identification of genes in the test biological sample that are aberrantly expressed (e.g., categorized as VERY LOW, LOW, HIGH, or VERY HIGH according to methods disclosed herein).

In some embodiments, normalization methods disclosed herein can allow the expression level of a gene or each gene within a test biological sample to be compared to reference ranges for normal tissues and/or to reference ranges for cohorts of tumors with known diagnosis and/or treatment outcomes (e.g., responsiveness to a cancer therapy or suitability for a clinical trial).

In some embodiments, normalization or calculating a normalized gene expression value can comprise subsampling to a target gene expression count per sample as disclosed herein. In some embodiments, normalization or calculating a normalized gene expression value can comprise a normalization calculation (e.g., quantile normalization calculation) as disclosed herein. In some embodiments, normalization or calculating a normalized gene expression value can comprise a scaling and/or transformation step as disclosed herein. Normalizing or calculating a normalized gene expression value can comprise subsampling of gene expression counts. Normalizing or calculating a normalized gene expression value can comprise subsampling to a target number of assigned reads or a minimum number of assigned reads per sample. An assigned read can be a sequencing read that is assigned to a gene or transcript. For example, an assigned read can be an RNA sequencing read that is aligned to a gene or transcript and included in a gene expression count for that gene or transcript.

Gene expression counts of a test biological sample can be subsampled. Gene expression counts of a control biological sample can be subsampled. In some embodiments the gene expression counts of all control biological samples and the test biological sample are each subsampled to the same read depth. For example, if X assigned reads are obtained from a sample, then Y reads are selected at random by subsampling to represent that sample, where Y<X. The same can be done for all control and all test (e.g., putative aberrant) samples so that Y is the same for all control samples and test samples, such that, e.g., all are subsampled to the same read depth before further processing and comparative analysis. In some embodiments, subsampling can correct for biases, for example, based on library size.

In some embodiments, gene expression counts are subsampled to a target number of assigned reads that is about 100,000, about 500,000, about 1 million, about 2 million, about 3 million, about 4 million, about 5 million, about 6 million, about 7 million, about 8 million, about 9 million, about 10 million, about 11 million, about 12 million, about 13 million, about 14 million, about 15 million, about 20 million, or about 25 million assigned reads per sample.

In some embodiments, gene expression counts are subsampled to a target number of assigned reads that is at least about 100,000, at least about 500,000, at least about 1 million, at least about 2 million, at least about 3 million, at least about 4 million, at least about 5 million, at least about 6 million, at least about 7 million, at least about 8 million, at least about 9 million, at least about 10 million, at least about 11 million, at least about 12 million, at least about 13 million, at least about 14 million, at least about 15 million, at least about 20 million, or at least about 25 million assigned reads per sample.

In some embodiments, gene expression counts are subsampled to a target number of assigned reads that is at most about 1 million, at most about 2 million, at most about 3 million, at most about 4 million, at most about 5 million, at most about 6 million, at most about 7 million, at most about 8 million, at most about 9 million, at most about 10 million, at most about 11 million, at most about 12 million, at most about 13 million, at most about 14 million, at most about 15 million, at most about 20 million, or at most about 25 million assigned reads per sample.

Several approaches can be suitable to normalizing gene expression data in accordance with one or more embodiments of the present disclosure. When the gene expression profiles to be normalized comprise global gene expression profiles with larger numbers (e.g., thousands) of genes, the statistical properties of a semi-continuous distribution can be used to normalize expression levels between samples. An example of such an approach to normalizing distributions is quantile normalization, which can be applied to normalize sets of global expression profiles. An additional example is Trimmed Measure of Means (TMM) normalization, which can be effective for gene expression data sets where large fluctuations in the values of a small percentage of individual genes occur.

In some embodiments, a method of the disclosure utilizes quantile normalization to generate normalized gene expression values. In some embodiments, a method of the disclosure does not utilize quantile normalization to generate normalized gene expression values. In some embodiments, a method of the disclosure utilizes TMM normalization to generate normalized gene expression values. In some embodiments, a method of the disclosure does not utilize TMM normalization to generate normalized gene expression values.

In some embodiments, normalizing or calculation of normalized gene expression values comprises quantile normalization. The quantile normalization can be performed on subsampled gene expression counts. For example, gene expression counts of all samples in the quantile normalization can be subsampled to a target number of assigned reads as disclosed herein (e.g., 1 million or 6 million), thereby generating subsampled gene expression counts. This subsampling can be done for a test biological sample and for each of a plurality of control biological samples. For each sample, the subsampled gene expression counts (e.g., non-zero subsampled gene expression counts) can be sorted by the total of gene expression counts assigned to each gene, for instance, from highest count to lowest count, or from lowest count to highest count (e.g., before subsampling or after subsampling). An average gene expression value for each position of the sorted gene expression counts can be calculated. The average gene expression value can be calculated from an average of all samples, for example, from an average of: (i) gene expression count at the position of the sorted gene expression counts of the test biological sample; and (ii) gene expression count for each of the plurality of control biological samples at a corresponding position of the sorted gene expression counts of the control biological sample. For example, a mean is calculated for the lowest gene expression count in all samples, a mean is then calculated for the 2nd lowest gene expression count in all samples, etc. A list of ordered average gene expression values calculated from all samples can thus be generated. The gene expression count at the sorted position for each sample can then be updated to be the average gene expression value for the sorted position. For example, the lowest gene expression count in a sample can be updated to be (e.g., replaced by) the lowest ordered average, the second lowest gene expression count is replaced by the second lowest ordered average, etc. This method can result in normalized gene expression values, e.g., that are suitable for comparison to a database.

In some embodiments, normalizing or calculation of normalized gene expression values comprises scaling and/or transformation. In some embodiments, a scaling factor is be applied to gene expression values that were calculated as disclosed herein, e.g., by quantile normalization. In some embodiments, the gene expression values can be divided by the scaling factor. In some embodiments, the scaling factor is calculated using a third quartile (Q3) value of the normalized gene expression values of the biological sample (e.g., test biological sample or control biological sample) that is being scaled. In some embodiments, gene expression values are multiplied by a scalar, for example, 10, 100, or 1,000. In some embodiments, gene expression values are log transformed, for example, log 2 transformed, or log 10 transformed.

An illustrative scaling factor can be calculated by ranking gene expression for each sample. The 75^{th} percentile/third quartile (Q3) for each sample can be used to calculate a mean (Q3_mean) of all the samples. The scaling factor can then be calculated using the following equation:

f_s=(Q3_mean*1,000)+1.

All normalized gene expression values can be divided by the scaling factor f_s, and resulting values log transformed (e.g., log 2 transformed). After log 2 transformation, the majority of normalized gene expression values can fall within a 0 to 20 point scale.

After quantile normalization, the third quantile of each normalized gene expression count dataset (e.g., table) can be set to a certain value, e.g., 1,000. When the resulting data are plotted on a log 2 scale (e.g., divided by a scaling factor and log 2 transformed, the expression values for many human genes can be generally between 0 and 20. In some embodiments, the log 2 expression levels for reference genes ACTB and IPO8 are about 17 and about 11, respectively, in breast, lung, colon, ovary, and many other tissue types; Her2 mRNA in normal breast tissue is about 12; and Her2 mRNA is from about 14-18 in Her2 positive tumors.

In some embodiments, a method disclosed herein utilizes a non-parametric statistical method or test. In some embodiments, a method disclosed herein does not utilize a non-parametric statistical method or test. In some embodiments, a method disclosed herein utilizes a parametric statistical method or test. In some embodiments, a method disclosed herein does not utilize a parametric statistical method or test.

In some embodiments, a normalization method disclosed herein does not model expression to probability distributions, such as a negative binomial or Poisson distribution. In some embodiments, a normalization method disclosed herein models expression to probability distributions, such as a negative binomial or Poisson distribution.

In some embodiments, normalization in a method of the disclosure does not involve internal controls. In some embodiments, normalization comprises use of internal controls, such as housekeeping genes. Certain genes can be ubiquitously or stably expressed at consistent levels, e.g., throughout multiple human tissue types, and/or in the presence and absence of a disease. The measured expression of one or more such reference gene(s) can serve as an internal control and used to correct for variations in the amount of input mRNA and other bias-free sources of variation between analyses.

In some embodiments, normalization comprises use of external controls, for example, spike in controls, such as adding gene-specific controls of known concentration to the sample. Each control can be substantially similar to a target sequence such that the control is amplified and sequenced with the same or a similar efficiency as the target sequence. In some embodiments, normalization in a method of the disclosure does not involve adding external, spike-in, and/or gene-specific controls of known concentration to the sample.

In some embodiments, gene expression values normalized by a method disclosed herein are validated against, for example, clinical data, immunohistochemistry data, q-RT-PCR data, an experimental dataset, or a simulated dataset.

Normalized gene expression values can comprise data for any type of RNA. In some embodiments, normalized gene expression values comprise data for mRNAs. In some embodiments, normalized gene expression values comprise data for non-coding RNAs. In some embodiments, normalized gene expression values comprise data for coding RNAs. In some embodiments, normalized gene expression values comprise data for micro RNAs.

In some embodiments, normalized gene expression values calculated by a method disclosed and the methods of generating the normalized gene expression values exhibit superiority over other normalization approaches, for example, approaches that utilize Reads Per Kilobase of transcript, per Million mapped reads (RPKM/TPM), trimmed mean of M values (TMM, e.g., edgeR, NIOSeq), RLE (relative loge expression, e.g., DESeq2). For example, methods disclosed herein can achieve superior concordance with protein expression levels (e.g., measured via immunohistochemistry, such as superior sensitivity or specificity of identification of aberrant gene expression as disclosed herein), superior ability to integrate data from multiple sources, superior ability to compare gene expression from a test biological sample (e.g., a single sample) to control biological samples (e.g., from normal individuals), or a combination thereof.

### Identification of Aberrantly Expressed Genes

Methods of the disclosure can comprise identifying genes that are expressed at aberrant (e.g., relatively high or low) levels. For example, one or more genes can be identified that are aberrantly expressed in a test biological sample relative to a plurality of control biological samples.

The aberrantly expressed gene(s) can be identified by a non-parametric comparison of (i) a normalized gene expression value for a candidate gene from the test biological sample, with (ii) a distribution of normalized gene expression values for the candidate gene obtained from the plurality of control biological samples.

Methods disclosed herein can facilitate more accurate identification of aberrantly expressed genes in a clinically-useful context, for example, from a single clinical sample without requiring cohorts and replicates. In some embodiments, methods disclosed herein allow an aberrantly expressed gene to be identified from a single test biological sample, for example, without obtaining or analyzing gene expression counts or normalized gene expression values from a biological sample of a second subject that has a disease.

Methods disclosed herein can facilitate more accurate identification of aberrantly expressed genes without requiring a matched normal sample or normal adjacent sample from the test subject. In some embodiments, methods disclosed herein allow an aberrantly expressed gene to be identified from a single test biological sample, for example, without analyzing gene expression counts obtained from a second biological sample from a control tissue of the test subject, such as an adjacent normal biological sample or a second biological sample that is considered normal (e.g., without a blood sample or PBMC sample for a non-hematologic cancer).

Methods disclosed herein can facilitate more accurate identification of aberrantly expressed genes without requiring replicates, for example, biological or technical replicates of the test biological sample.

Methods disclosed herein can facilitate more accurate identification of aberrantly expressed genes without requiring groups or cohorts. In some embodiments, identifying a gene that is aberrantly expressed does not include comparing gene expression counts or normalized gene expression values from (i) a first cohort comprising the test subject and at least one additional subject to (ii) a second cohort comprising at least two subjects. In some embodiments, identifying a gene that is aberrantly expressed does not include comparing gene expression counts or normalized gene expression values from (i) a first cohort comprising the test subject and at least two additional subjects to (ii) a second cohort comprising at least three subjects. In some embodiments, identifying a gene that is aberrantly expressed does not include comparing gene expression counts or normalized gene expression values from (i) a first cohort comprising the test subject and at least four additional subject to (ii) a second cohort comprising at least five subjects. In some embodiments, identifying a gene that is aberrantly expressed does not include comparing gene expression counts or normalized gene expression values from (i) a first cohort comprising the test subject and at least nine additional subject to (ii) a second cohort comprising at least ten subjects.

After normalized gene expression values are obtained for control biological samples, a reference range can be determined for a control RNA transcription level of one or more genes. Reference ranges can be calculated for all genes. The reference ranges can be calculated for all clinically significant genes, e.g., in the normal tissue's expression profiles. A reference range can comprise an upper and lower limit such that the majority of normalized gene expression values for the control biological sample for that gene fall between these limits. Normalized gene expression values that fall between the upper and lower limit can be categorized normal expression values. Normalized gene expression values that fall outside the upper and lower limit can be categorized aberrant expression values, for example, are greater the upper limit, greater than or equal to the upper limit, less than the lower limit, or less than or equal to the lower limit.

In some embodiments, the upper limit of the reference range for a candidate gene can be a normalized gene expression value that is greater than a sum of median plus two times interquartile range (IQR) of the normalized gene expression values for the candidate gene in the plurality of control biological samples.

In some embodiments, the lower limit of the reference range for a candidate gene can be a normalized gene expression value that is less than a difference of median and two times IQR of the normalized gene expression values for the candidate gene in the plurality of control biological samples.

In some embodiments, normalized gene expression values of a test biological sample are categorized, wherein categories comprise VERY LOW, LOW, NORMAL, HIGH, and VERY HIGH categories, wherein:
the VERY HIGH category includes genes with a normalized gene expression value for the test biological sample that is greater than a threshold calculated based on distribution of a candidate gene's expression in the plurality of control biological samples and is lesser of (i) a maximum normalized gene expression value for the candidate gene in the plurality of control biological samples; and (ii) a sum of third quartile (Q3) and 1.5 times interquartile range (IQR) of normalized gene expression values for the candidate gene in the plurality of control biological samples;
the HIGH category includes genes not classified in the VERY HIGH category with a normalized gene expression value for the test biological sample that is greater than a sum of median plus two times IQR of the normalized gene expression values for the candidate gene in the plurality of control biological samples;
the VERY LOW category includes genes with a normalized gene expression value for the test biological sample that is less than a threshold calculated based on distribution of the candidate gene's expression in the plurality of control biological samples and is lesser of: (i) minimum normalized gene expression value for the candidate gene in the plurality of control biological samples; and (ii) a difference of first quartile (Q1) and 1.5 times IQR of the normalized gene expression values for the candidate gene in the plurality of control biological samples;
the LOW category includes genes not classified in the VERY LOW category with a normalized gene expression value for the test biological sample that is: less than a difference of median and two times IQR of the normalized gene expression values for the candidate gene in the plurality of control biological samples; and
the NORMAL category is assigned to genes that are not categorized in the VERY LOW, LOW, HIGH, or VERY HIGH categories.

In some embodiments, normalized gene expression values of a test biological sample are categorized, wherein categories comprise VERY LOW, LOW, NORMAL, HIGH, and VERY HIGH categories, wherein thresholds for the categories are calculated according to a non-parametric comparison of (a) a normalized gene expression value for a candidate gene in the test biological sample with (b) a distribution of normalized gene expression values for the candidate gene obtained from the plurality of control biological samples using equation 1, wherein: (i) yᵢⱼ represents expression of gene j in sample I; (ii) medianₙⱼ is a median expression level for gene j in the plurality of control biological samples; (iii) yₙⱼₘₐₓ is maximum expression of gene j in the plurality of control biological samples; (iv) yₙⱼₘᵢₙ is minimum expression of gene j in the plurality of control biological samples; (v) Q₁ₙⱼ is a first quartile of gene j expression in the plurality of control biological samples; (vi) Q₃ₙⱼ is a third quartile of gene j expression in the plurality of control biological samples; (vii) IQRₙⱼ is an interquartile range of gene j expression in the plurality of control biological samples; and (viii) rₙⱼ is a range of expression of gene j in the plurality of control biological samples and is calculated using equation 2.

Equation 1 can be:

$$f ( {y}_{ij} ) = \{ \begin{matrix}VERY HIGH , & if {y}_{ij} > {median}_{nj} + 2 * {r}_{nj} \\ HIGH , & if {y}_{ij} > min ( {y}_{{nj}_{max}} , {Q}_{{3}_{nj}} + 1.5 * {IQR}_{nj} ) \\ LOW , & if {y}_{ij} < max ( {y}_{{nj}_{min}} , {Q}_{{1}_{nj}} - 1.5 * {IQR}_{nj} ) \\ VERY LOW , & if {y}_{ij} < {median}_{nj} - 2 * {r}_{nj}\end{matrix}$$

Equation 2 can be:

rₙⱼ=min(yₙⱼ_{ₘₐₓ},Q₃_{ₙⱼ}+1.5*IQRₙⱼ)−max(yₙⱼ_{ₘᵢₙ},Q₁_{ₙⱼ}−1.5*IQRₙⱼ)

Methods disclosed herein that utilize RNA seq can allow a large number of genes to be concurrently evaluated for aberrant expression. Any suitable number of genes can be identified that are aberrantly expressed in the test biological sample relative to the plurality of control biological samples. In some embodiments, one aberrantly expressed gene is identified. In some embodiments, one or more aberrantly expressed genes is/are identified. In some embodiments, two or more, three or more, four or more, five or more, six or more, seven or more, eight or more, nine or more, 10 or more, 15 or more, 20 or more, 25 or more, 30 or more, 50 or more, 75 or more, or 100 or more aberrantly expressed genes are identified.

Multiple statistical parameters can be used to describe the spread of a data distribution.

In some embodiments, the reference range is computed for each gene using a fully empirical data model. Expression levels for many genes in biological samples, even samples from the same tissue, do not follow a normal distribution in some cases. For instance, genes that encode tumor specific antigens such as the MAGEA and MAGEB family of antigens are not expressed at detectable levels in many noncancerous tissues. However, many tumor samples express MAGE family genes at significant levels. These genes have a zero-inflated expression distribution such that the mean expression level and lower limit are both zero, but have a non-zero upper limit.

Diverse distributions are sometimes depicted in the scientific literature as boxplots. Boxplot statistics can comprise a mean or median, inter quartile range, and outer limits which are referred to as upper and lower whiskers. According to the Tukey method, the lower limit can be the lowest data point still within 1.5 IQR of the lower quartile (Q1), where IQR is the interquartile range calculated as the difference between the 3rd quartile (Q3) and 1st quartile (Q1) of the data. Similarly, the upper limit can be the highest datum still within 1.5 IQR of the upper quartile.

In some embodiments, the upper and lower limits for a control RNA transcription level of one or more genes is determined by the upper and lower whiskers of the Tukey boxplot for normalized gene expression values of the one or more genes in a group of control biological samples. In some embodiments, the upper and lower limits are the 98th percentile and 2nd percentile of the reference distribution, respectively. In some embodiments, the upper and lower limits are the 95th percentile and 5th percentile of the reference distribution, respectively.

In some embodiments, the thresholds that determine the normal and aberrant reference ranges are adjusted as additional information becomes available. In some embodiments, the control RNA transcription level of all genes measured in the expression profile of a biological sample are compared to the upper and lower limits that are determined using the same quantile or percentile across all genes. In some embodiments, the control (e.g., normal) RNA transcription levels of all genes measured in the expression profile of a biological sample are compared to upper and lower limits that are determined by unique quantiles or percentiles depending upon the behavior of the one or more genes in test biological sample and control biological samples respectively. Optionally, outcome data is factored into the determination.

In some embodiments, identifying an aberrantly expressed gene utilizes anon-parametric statistical method or test. In some embodiments, a non-parametric statistical method or test has a higher accuracy (e.g., a lower false discovery rate in a study), is less sensitive to outliers, or a combination thereof. In some embodiments, identifying an aberrantly expressed gene does not utilize a non-parametric statistical method or test. In some embodiments, identifying an aberrantly expressed gene utilizes a parametric statistical method or test. In some embodiments, identifying an aberrantly expressed gene does not utilize a parametric statistical method or test.

In some embodiments, identifying an aberrantly expressed gene does not include modelling expression to probability distributions, such as a negative binomial or Poisson distribution. In some embodiments, identifying an aberrantly expressed gene models expression to probability distributions, such as a negative binomial or Poisson distribution.

In some embodiments, a RNA transcription level of one or more genes in a test biological sample that are expressed at levels above the upper limit of a reference range of a control RNA transcription level is identified as being over-expressed, while a RNA transcription level of one or more genes in a test biological sample that are expressed at levels below the lower limit of the reference range of a control RNA transcription level is identified as being under-expressed. Accordingly, a RNA transcription level that falls in between the upper and lower limits can be categorized as being expressed at normal levels or within the normal range. In some embodiments, additional levels of expression can be assigned, e.g., low, very low, high, and very high, e.g., as disclosed herein.

An average or mean disclosed herein can be, for example, an arithmetic mean, a geometric mean, a harmonic mean, or a median. In some embodiments, an average or mean is an arithmetic mean. In some embodiments, an average or mean is a geometric mean. In some embodiments, an average or mean is a harmonic mean. In some embodiments, an average or mean is a median.

### Wellness Recommendation, Prognosis, and Diagnosis

Normalized gene expression values and aberrantly expressed genes identified as disclosed herein can be useful to identify associations and provide various recommendations and predictions. For example, a method of the present disclosure can comprise providing a wellness recommendation, treatment recommendation, prediction of response to therapeutic agent or regimen, diagnosis, prognosis, and/or outcome prediction.

A wellness recommendation can comprise a treatment recommendation. In some embodiments, a wellness recommendation does not include a treatment recommendation. In some embodiments, a wellness recommendation does not include administering a therapeutic agent. For example, in some embodiments, a wellness recommendation comprises a recommendation related to lifestyle, diet, nutrition, dietary supplementation, physical activity, exercise, alcohol consumption, early screening for a disease, or allergy or intolerance to a certain food, nutrient, or metabolite. In some embodiments, a wellness recommendation comprises a recommendation for an intervention that modulates expression or activity of a product encoded by a gene that is aberrantly expressed, for example, a recommendation related to lifestyle, diet, nutrition, dietary supplementation, physical activity, exercise, alcohol consumption, or allergy or intolerance to a certain food, nutrient, or metabolite.

A treatment recommendation can comprise a recommendation to administer a therapeutic agent to a subject. A treatment recommendation can comprise a recommendation not to administer a therapeutic agent to a subject. A treatment recommendation can comprise recommending participation of a subject in a clinical trial that the subject is a candidate for and may benefit from. A treatment recommendation can comprise recommending a treatment regimen, for example, a number of doses of a therapeutic agent, a dosing frequency of a therapeutic agent, and/or a duration of administration of a therapeutic agent. A treatment recommendation can comprise a combination therapy, for example, a combination of any two therapeutic agents, such as any two therapeutic agents disclosed herein.

The relationship between the gene expression states, disease, and clinical actionability can be complex. Methods of the disclosure can comprise providing a wellness recommendation, such as a treatment recommendation, based on a gene expression profile that comprises, for example, normalized gene expression values and/or genes identified as aberrantly expressed. The aberrantly expressed genes can be under-expressed, such as genes categorized in “LOW” and/or “VERY LOW” categories, over-expressed, such as genes categorized in “HIGH” and/or “VERY HIGH” categories, or a combination of under-expressed and over-expressed genes.

Aberrantly expressed genes can be identified as disclosed herein. For example, if a normalized gene expression value of a test biological sample (e.g., tumor sample) crosses one or more thresholds derived from the distribution of gene expression levels in a plurality of control (e.g., normal and/or healthy) biological samples, a gene can be identified as aberrantly expressed. This comparison can be used, e.g., rather than assigning significance to the magnitude of the change in RNA transcription level from a single reference level. In some embodiments, the expression levels of one or more genes in a test biological sample can be compared to the reference ranges for the same in a population of diseased tissues, bodily fluids, or other biological samples. Based on this comparison, a discrete state can be assigned to each gene based its relationship to one or more expression thresholds defined according to the methods described herein (e.g., VERY LOW, LOW, NORMAL, HIGH, or VERY HIGH).

In some embodiments, over-expression (e.g., categorized as “HIGH” or “VERY HIGH”) of a gene identified by methods of the disclosure can be used to identify a therapeutic agent, regimen, combination therapy, or clinical trial that could benefit a subject that the test biological sample is from. In some embodiments, under-expression (e.g., categorized as “LOW” or “VERY LOW”) of a gene identified my methods of the disclosure can be used to identify a therapeutic agent, regimen, combination therapy, or clinical trial that could benefit a subject that provided the test biological sample

Any gene or combination of genes can be used to identify the therapeutic agent, regimen, combination therapy, or clinical trial. For example, pembrolizumab is an approved immune checkpoint inhibitor that is approved in non-small cell lung cancer for tumors that have high PD-L1 expression. Accordingly, a treatment recommendation can comprise administering an anti-PD-L1 agent such as pembrolizumab where PD-L1 is detected as expressed (e.g., over-expressed, such as at HIGH or VERY HIGH level disclosed herein). A treatment recommendation can comprise not administering an anti-PD-L1 agent if low levels of PD-L1 are expressed, or if PD-L1 expression is not detected.

In another example, the proliferation marker Ki-67 (encoded by the gene MKI67) has been used as a prognostic marker for breast cancer, where higher levels can indicate more aggressive disease. A relatively more aggressive therapeutic agent or treatment regimen can be recommended when high expression of MK167 is detected.

Methods of the disclosure can comprise identifying a clinical trial (e.g., identifying a subject as a candidate for the clinical trial) based on normalized gene expression values and/or genes identified as aberrantly expressed. For example, immunotherapies to treat cancers that over-express carcinoembryonic antigen (CEA) are being tested in ongoing clinical trials, e.g., NCT02650713 and NCT02850536. In one example, such a clinical trial can be identified or a test subject identified as a candidate for such a clinical trial based on aberrant over-expression of CEA (e.g., at a HIGH or VERY HIGH level disclosed herein).

Any gene or combination of genes can be used to identify the clinical trial or identify a subject as a candidate for the clinical trial. For example, defects in DNA repair pathway genes, including BRCA 1/2, ATM and PTEN, can enhance tumor response to treatment with PARP inhibitors, and these defects can manifest as deletion or silencing of pathway genes. The utility of this approach can be illustrated by the TOPARP-A phase II trial of olaparib in prostate cancer, where all seven patients with BRCA2 silencing responded to the treatment. Similarly, under-expression of MGMT in glioblastoma can be associated with an enhanced likelihood of response to temozolimide.

Normalized gene expression values and/or aberrantly expressed genes (e.g., patterns thereof/gene signatures that comprise multiple gene expression values and/or aberrantly expressed genes) for specific cancers can correlate with prognoses for therapeutic agents and/or treatment regimens.

A gene that is aberrantly expressed can be associated with an increased likelihood of a favorable response to a therapeutic agent. A gene that is aberrantly expressed can be associated with a decreased likelihood of a favorable response to a therapeutic agent. A combination of aberrantly expressed genes can be associated with an increased likelihood of a favorable response to a therapeutic agent. A combination of aberrantly expressed genes can be associated with a decreased likelihood of a favorable response to a therapeutic agent.

A normalized gene expression value can be associated with an increased likelihood of a favorable response to a therapeutic agent. A normalized gene expression value can be associated with a decreased likelihood of a favorable response to a therapeutic agent. A combination of normalized gene expression values can be associated with an increased likelihood of a favorable response to a therapeutic agent. A combination of normalized gene expression values can be associated with a decreased likelihood of a favorable response to a therapeutic agent.

For example, a patient having triple-negative breast cancer, i.e., ER-/PR-/HER2-cancer, has a different prognosis for treatment with a drug that is capable of targeting ER and PR, e.g., tamoxifen, than does a comparable patient having a breast cancer with at least one positive signal between the ER and PR genes.

By matching the normalized gene expression values and/or aberrantly expressed genes (e.g., patterns thereof/gene signatures) of test biological sample from a subject to a potential therapeutic agent or treatment regimen, methods of the disclosure can provide a treatment recommendation and/or a clinical outcome predictor for the therapeutic agent or treatment regimen. In such cases, methods of the disclosure can identify therapeutic agents, regimens, combination therapies, clinical trials, etc., that a subject is most likely to respond to or not respond to.

Methods disclosed herein can comprise identification of therapeutic agents, and treatment recommendations for therapeutic agents, for example, based on one or more normalized gene expression values and/or aberrantly expressed genes. In some embodiments, methods of the disclosure comprise identifying a suitable therapeutic agent that can benefit a subject in need thereof (e.g., be administered to the subject). In some embodiments, methods of the disclosure comprise identifying a therapeutic agent that is unlikely to benefit a subject in need thereof (e.g., be administered to the subject). Methods can characterize administration of a therapeutic agent as unnecessary based on one or more normalized gene expression values and/or aberrantly expressed genes, for example, a recommendation to withhold chemotherapy can be made based on a risk profile associated with a gene expression profile.

Non-limiting examples of therapeutic agents include vaccines (e.g., mRNA vaccines), AKT inhibitors, alkylating agents, anti-angiogenic agents, antibiotic agents, antifolates, anti-hormone therapies, anti-inflammatory agents, antimetabolites, anti-VEGF agents, apoptosis promoting agents, aromatase inhibitors, ATM regulators, biologic agents, BRAF inhibitors, BTK inhibitors, CAR-T cells, CAR-NK cells, CDK inhibitors, cell growth arrest inducing-agents, cell therapies, chemotherapy, cytokine therapies, cytotoxic drugs, demethylating agents, differentiation-inducing agents, estrogen receptor antagonists, gene therapy agents, growth factor inhibitors, growth factor receptor inhibitors, HDAC inhibitors, heat shock protein inhibitors, hematopoietic stem cell transplantation (HSCT), hormones, hydrazine, immune checkpoint inhibitors, immumomodulators, immunosuppressants, kinase inhibitors, KRAS inhibitors, matrix metalloproteinase inhibitors, MEK inhibitors, mitotic inhibitors, mTOR inhibitors, multi-specific (e.g., bispecific) immune cell engagers, multi-specific (e.g., bispecific) killer cell engagers, multi-specific (e.g., bispecific) T cell engagers, nitrogen mustards, oncolytic viruses, oxazaphosphorines, p53 reactivating agents, plant alkaloids, platinum-based agents, proteasome inhibitors, purine analogs, purine antagonists, pyrimidine antagonists, radiation therapy, ribonucleotide reductase inhibitors, signal transduction inhibitors, RNA silencing (e.g., RNAi) agents, gene editing agents, a CRISPR/Cas systems or a component thereof, an RNA replacement therapy, a protein replacement therapy, a gene therapy, antibody drug conjugates, surgery, taxanes, therapeutic antibodies, topoisomerase inhibitors, transgenic T cells, tyrosine kinase inhibitors, and vinca alkaloids.

A therapeutic agent can be, for example, an anti-cancer therapeutic. Non-limiting examples of anti-cancer therapeutic agents include cancer vaccines (e.g., mRNA vaccines), AKT inhibitors, alkylating agents, anti-angiogenic agents, antibiotic agents, antifolates, anti-hormone therapies, anti-inflammatory agents, antimetabolites, anti-VEGF agents, apoptosis promoting agents, aromatase inhibitors, ATM regulators, biologic agents, BRAF inhibitors, BTK inhibitors, CAR-T cells, CAR-NK cells, CDK inhibitors, cell growth arrest inducing-agents, cell therapies, chemotherapy, cytokine therapies, cytotoxic drugs, demethylating agents, differentiation-inducing agents, estrogen receptor antagonists, gene therapy agents, growth factor inhibitors, growth factor receptor inhibitors, HDAC inhibitors, heat shock protein inhibitors, hematopoietic stem cell transplantation (HSCT), hormones, hydrazine, immune checkpoint inhibitors, immumomodulators, kinase inhibitor, KRAS inhibitors, matrix metalloproteinase inhibitors, MEK inhibitors, mitotic inhibitors, mTOR inhibitors, multi-specific (e.g., bispecific) immune cell engagers, multi-specific (e.g., bispecific) killer cell engagers, multi-specific (e.g., bispecific) T cell engagers, nitrogen mustards, oncolytic viruses, oxazaphosphorines, p53 reactivating agents, plant alkaloids, platinum-based agents, proteasome inhibitors, purine analogs, purine antagonists, pyrimidine antagonists, radiation therapy, ribonucleotide reductase inhibitors, signal transduction inhibitors, RNA silencing (e.g., RNAi) agents, gene editing agents, a CRISPR/Cas systems or a component thereof, an RNA replacement therapy, a protein replacement therapy, a gene therapy, antibody drug conjugates, surgery, taxanes, therapeutic antibodies, topoisomerase inhibitors, transgenic T cells, tyrosine kinase inhibitors, and vinca alkaloids.

A therapeutic agent can be a drug. A therapeutic agent can be a non-cancer therapeutic, for example, a therapeutic for a metabolic disease, autoimmune disease, neurological disease, or degenerative disease. A therapeutic agent can be, for example, a vaccine (e.g., cancer vaccine), a drug, an immunotherapy, an immune checkpoint inhibitor, a kinase inhibitor, a small molecule, a chemotherapeutic agent, a radiotherapy, a biologic, or any combination thereof.

A therapeutic agent can modulate (e.g., increase or decrease) activity of a target gene (e.g., an aberrantly expressed gene), or a product encoded by the target gene, such as a protein or RNA. A therapeutic agent can modulate (e.g., increase or decrease) expression of a target gene (e.g., an aberrantly expressed gene). A therapeutic agent can modulate (e.g., increase or decrease) activity of a ligand or receptor of a target gene (e.g., an aberrantly expressed gene). In some embodiments, a therapeutic agent can alter the gene product of an aberrantly-expressed gene, e.g., by targeting the gene product, the transcript of the gene, or epigenetic factors that influence a property of the gene (e.g., expression). Non-limiting examples include targeting the protein that the gene encodes, reducing expression levels of the gene using gene therapy or RNAi, and using RNA vaccines to establish an immune response.

Methods of the disclosure can be used to identify a therapeutic agent that can be used in the treatment of a disease or condition, such as a cancer.

In some embodiments, a method of aiding in a treatment of a cancer in a test subject includes: (a) quantifying a RNA transcription level of one or more genes in a test sample from test subject, (b) comparing the RNA transcription level of the one or more genes in the test subject to a control RNA transcription level (e.g., from a plurality of control biological subjects), and (c) providing a treatment recommendation for the cancer in the subject if the RNA transcription level is different from the control RNA transcription level. The treatment recommendation can comprise administering a therapeutic agent (e.g., drug) capable of modifying the RNA transcription level of the one or more genes, e.g., to be more similar to the control RNA transcription level. In some embodiments, the therapeutic agent (e.g., drug) is capable of directly or indirectly modifying the amount of the gene expressed at RNA and/or protein level. A therapeutic agent that is capable of modifying the RNA transcription level can be an agent that is designed to effect changes in a specific gene product, or an agent that possess the characteristic of having an effect of a RNA transcription level of one or more genes without explicit design for such purpose.

Certain therapeutic agents, such as anti-cancer drugs, e.g., tamoxifen, are known to reduce the RNA transcription level of the ER gene. Hence, an ER+ cancer can be responsive to tamoxifen. In some embodiments, a method of the present disclosure comprises identifying a biological sample having higher level of ER RNA expression than a control level, and reporting that the corresponding cancer can be responsive to tamoxifen.

In some embodiments, the therapeutic agent is capable of modulating the functional activity of the gene at RNA and/or protein level, e.g., promoting or inhibiting function of the gene or protein. In some embodiments, the drug can target the protein product encoded by the RNA, for example, an immune checkpoint inhibitor (e.g., nivolumab) can bind to and inhibit the activity of an immune checkpoint protein (e.g., PD-1), thereby increasing an anti-cancer immune response. In some embodiments, the therapeutic agent does not alter an expression level (e.g., an RNA expression level) of the gene that is identified as aberrantly expressed.

A treatment or regimen disclosed herein can comprise administering a therapeutic agent capable of modifying the RNA transcription level of the gene to the control RNA transcription level. The drug can be capable of directly or indirectly modifying the RNA transcription level and/or the protein translation level of the one or more genes to the control RNA transcription level. For example, the drug can target the protein product encoded by the RNA. In some embodiments, the method comprises providing a report identifying a drug capable of modifying the RNA transcription level of the gene to the control RNA transcription level. In some embodiments, the gene is ER, PR, or ESR1 and the drug is tamoxifen. In some embodiments, the gene is PD-1 and the drug is nivolumab or ipilumimab. The report can comprise any suitable therapeutic agent associated with an expression level of one or more genes.

A therapeutic agent can be an immune checkpoint modulator, such as an immune checkpoint inhibitor. Non-limiting examples of immune checkpoint modulators include PD-L1 inhibitors such as durvalumab (Imfinzi) from AstraZeneca, atezolizumab (MPDL3280A) from Genentech, avelumab from EMD Serono/Pfizer, CX-072 from CytomX Therapeutics, FAZ053 from Novartis Pharmaceuticals, KN035 from 3D Medicine/Alphamab, LY3300054 from Eli Lilly, or M7824 (anti-PD-L1/TGFbeta trap) from EMD Serono; PD-L2 inhibitors such as GlaxoSmithKline's AMP-224 (Amplimmune), and rHIgM12B7; PD-1 inhibitors such as nivolumab (Opdivo) from Bristol-Myers Squibb, pembrolizumab (Keytruda) from Merck, AGEN 2034 from Agenus, BGB-A317 from BeiGene, B1-754091 from Boehringer-Ingelheim Pharmaceuticals, CBT-501 (genolimzumab) from CBT Pharmaceuticals, INCSHR1210 from Incyte, JNJ-63723283 from Janssen Research & Development, MEDI0680 from MedImmune, MGA 012 from MacroGenics, PDR001 from Novartis Pharmaceuticals, PF-06801591 from Pfizer, REGN2810 (SAR439684) from Regeneron Pharmaceuticals/Sanofi, or TSR-042 from TESARO; CTLA-4 inhibitors such as ipilimumab (also known as Yervoy®, MDX-010, BMS-734016 and MDX-101) from Bristol Meyers Squibb, tremelimumab (CP-675,206, ticilimumab) from Pfizer, or AGEN 1884 from Agenus; LAG3 inhibitors such as BMS-986016 from Bristol-Myers Squibb, IMP701 from Novartis Pharmaceuticals, LAG525 from Novartis Pharmaceuticals, or REGN3767 from Regeneron Pharmaceuticals; B7-H3 inhibitors such as enoblituzumab (MGA271) from MacroGenics; KIR inhibitors such as Lirilumab (IPH2101; BMS-986015) from Innate Pharma; CD137 inhibitors such as urelumab (BMS-663513, Bristol-Myers Squibb), PF-05082566 (anti-4-1BB, PF-2566, Pfizer), or XmAb-5592 (Xencor); and PS inhibitors such as Bavituximab.

Methods disclosed herein can comprise identification of a combination of therapeutic agents, and treatment recommendations for the combination of therapeutic agents, for example, based on one or more normalized gene expression values and/or aberrantly expressed genes. In some embodiments, methods of the disclosure comprise identifying a suitable combination of therapeutic agents that can benefit a subject in need thereof (e.g., be administered to the subject). In some embodiments, methods of the disclosure comprise identifying a combination of therapeutic agents that is unlikely to benefit a subject in need thereof (e.g., be administered to the subject). Methods can characterize administration of a combination of therapeutic agents as unnecessary based on one or more normalized gene expression values and/or aberrantly expressed genes, for example, a recommendation to withhold a combination of chemotherapeutic agents can be made based on a risk profile associated with a gene expression profile.

The combination of therapeutic agents can comprise any two therapeutic agents disclosed herein. The combination of therapeutic agents can comprise, for example, or more of cancer vaccines, AKT inhibitors, alkylating agents, anti-angiogenic agents, antibiotics, antifolates, anti-hormone therapies, anti-inflammatory agents, antimetabolites, anti-VEGF agents, apoptosis promoting agents, aromatase inhibitors, ATM regulators, biologic agents, BRAF inhibitors, BTK inhibitors, CAR-T cells, CDK inhibitors, cell growth arrest inducing-agents, cell therapies, chemotherapy, cytokine therapies, cytotoxic drugs, demethylating agents, differentiation-inducing agents, estrogen receptor antagonists, gene therapy agents, growth factor inhibitors, growth factor receptor inhibitors, HDAC inhibitors, heat shock protein inhibitors, hematopoietic stem cell transplantation (HSCT), hormones, hydrazine, immune checkpoint modulators (e.g., inhibitors), immumomodulators, kinase inhibitor, KRAS inhibitors, matrix metalloproteinase inhibitors, MEK inhibitors, mitotic inhibitors, mTOR inhibitors, multi-specific (e.g., bispecific) immune cell engagers, multi-specific (e.g., bispecific) killer cell engagers, multi-specific (e.g., bispecific) T cell engagers, nitrogen mustards, oncolytic viruses, oxazaphosphorines, p53 reactivating agents, plant alkaloids, platinum-based agents, proteasome inhibitors, purine analogs, purine antagonists, pyrimidine antagonists, radiation therapy, ribonucleotide reductase inhibitors, signal transduction inhibitors, surgery, taxanes, therapeutic antibodies, topoisomerase inhibitors, transgenic T cells, tyrosine kinase inhibitors, and vinca alkaloids.

Methods disclosed herein can comprise identification of cancer vaccine, and treatment recommendations for the cancer vaccine, for example, based on one or more normalized gene expression values and/or aberrantly expressed genes. In some embodiments, methods of the disclosure comprise identifying a suitable cancer vaccine that can benefit a subject in need thereof. In some embodiments, methods of the disclosure comprise identifying a cancer vaccine that is unlikely to benefit a subject in need thereof.

In some embodiments, methods of the disclosure comprise identifying a cancer vaccine that can benefit a subject, and/or designing a cancer vaccine de novo that can benefit a subject. The cancer vaccine can be a mRNA vaccine. The cancer vaccine can be a protein vaccine. The cancer vaccine can utilize a viral vector. The cancer vaccine can utilize a virus like particle. The cancer vaccine can utilize an adjuvant. The cancer vaccine can utilize a liposome (e.g., a fusogenic liposome). The cancer vaccine can utilize a nanoparticle. The cancer vaccine can utilize mRNA with one or more stabilizing modifications to the RNA. The cancer vaccine can utilize cells, e.g., antigen presenting cells, such as professional antigen presenting cells, dendritic cells, myeloid cells, monocytes, macrophages, or B cells. The cells can be autologous or allogeneic to the subject. The cells can be HLA matched to the subject.

mRNA vaccines combine the potential of mRNA to encode almost any protein with an excellent safety profile and a flexible production process that can be rapidly adjusted to incorporate sequences of interest. Once administered and internalized by host cells, the mRNA transcripts can be translated directly in the cytoplasm of the cell. The resulting antigens are presented to the immune system cells to stimulate an immune response. Dendritic cells (DCs) can be utilized as a carrier by delivering antigen mRNAs or total tumor RNA to the cytoplasm. Then the mRNA-loaded DCs can be delivered to the host to elicit a specific immune response.

An mRNA vaccine disclosed herein can comprise mRNA encapsulated into a carrier to protect the mRNA from degradation and to stimulate cellular uptake and endosomal escape thereof. In some embodiments, the mRNA vaccine comprises lipid nanoparticles. The lipid nanoparticle can comprise pH-responsive lipids; neutral helper lipids, such as zwitterionic lipid and/or sterol lipid (e.g., cholesterol) to stabilize the lipid bilayer of the lipid nanoparticle; a PEG-lipid to improve the colloidal stability in biological environments, and any combination thereof. In some embodiments, the mRNA vaccine comprises lipoplexes.

In some embodiments, methods of the disclosure comprise identifying a suitable combination of a cancer vaccine and a second therapeutic agent that can be administered to a subject in need thereof. The second therapeutic agent can comprise any one or more therapeutic agents disclosed herein, for example, of AKT inhibitors, alkylating agents, anti-angiogenic agents, antibiotics, antifolates, anti-hormone therapies, anti-inflammatory agents, antimetabolites, anti-VEGF agents, apoptosis promoting agents, aromatase inhibitors, ATM regulators, biologic agents, BRAF inhibitors, BTK inhibitors, CAR-T cells, CDK inhibitors, cell growth arrest inducing-agents, cell therapies, chemotherapy, cytokine therapies, cytotoxic drugs, demethylating agents, differentiation-inducing agents, estrogen receptor antagonists, gene therapy agents, growth factor inhibitors, growth factor receptor inhibitors, HDAC inhibitors, heat shock protein inhibitors, hematopoietic stem cell transplantation (HSCT), hormones, hydrazine, immune checkpoint modulators (e.g., inhibitors), immumomodulators, kinase inhibitor, KRAS inhibitors, matrix metalloproteinase inhibitors, MEK inhibitors, mitotic inhibitors, mTOR inhibitors, multi-specific (e.g., bispecific) immune cell engagers, multi-specific (e.g., bispecific) killer cell engagers, multi-specific (e.g., bispecific) T cell engagers, nitrogen mustards, oncolytic viruses, oxazaphosphorines, p53 reactivating agents, plant alkaloids, platinum-based agents, proteasome inhibitors, purine analogues, purine antagonists, pyrimidine antagonists, radiation therapy, ribonucleotide reductase inhibitors, signal transduction inhibitors, surgery, taxanes, therapeutic antibodies, topoisomerase inhibitors, transgenic T cells, tyrosine kinase inhibitors, and vinca alkaloids. In some embodiments, the second therapeutic agent is an immune checkpoint inhibitor.

With analysis of a normalized gene expression values of a test biological sample derived from a test subject, the instant methods can be used to provide a diagnosis. A diagnosis can be based on a normalized gene expression value, e.g., one normalized gene expression value or combination of normalized gene expression values. A diagnosis can be based on an aberrantly expressed gene, e.g., one aberrantly expressed gene or a combination of aberrantly expressed genes. A diagnosis can be based on a combination of one or more aberrantly expressed genes and one or more normalized gene expression values. The normalized gene expression values can include, for example, genes that are expressed at normal levels or are not identified as aberrantly expressed.

A method disclosed herein can be used to detect or diagnose a disease or condition, such as a cancer, if an aberrant expression of the one or more genes is correlated to a specific disease or condition. An aberrantly expressed gene can be expressed at a higher or lower level compared to control biological samples. An aberrantly expressed gene can be, for example, a normalized gene expression value that is categorized as “VERY LOW” “LOW” “HIGH or “VERY HIGH” according to methods disclosed herein.

Methods disclosed herein can comprise diagnosing a subject as having a cancer. The method can also be used to predict the development of cancer or risk of cancer based on identification of pre-cancerous lesions that are different from normal tissue.

A method disclosed herein can be used to detect or diagnose a disease or condition that is not cancer, such as a metabolic, autoimmune, neurological, or degenerative disease.

Sequencing the RNA can occur from the 3′-end, the 5′-end, or a combination thereof, e.g., non-discriminately. In some embodiments, the method of diagnosing a cancer comprises: (a) quantifying a RNA transcription level of a gene in a subject comprising: (i) extracting RNA from a test biological sample from the test subject, (ii) measuring the RNA using an RNA sequencing kit comprising: (1) sequencing the RNA from the 3′-end, and (2) identifying the RNA, (b) comparing the RNA transcription level of the gene in the subject to a control RNA transcription level, and (c) diagnosing the cancer if the RNA transcription level is different from the control RNA transcription level.

Methods disclosed herein that comprise providing a wellness recommendation, treatment recommendation, prediction of response to therapeutic agent or regimen, diagnosis, prognosis, and/or outcome prediction can comprise determining the RNA transcription level of any gene using the methods of the present disclosure, for example, as a normalized gene expression value.

In some embodiments, methods of the disclosure are used to quantify a transcription level (e.g., normalized gene expression value) of a tumor associated antigen (TAA), such as a cancer testis antigen (CTA). In some embodiments, methods of the disclosure are used to quantify a transcription level (e.g., normalized gene expression value) of a neoantigen. In some embodiments, methods of the disclosure are used to quantify a transcription level (e.g., normalized gene expression value) of a tumor specific antigen (TSA). In some embodiments, methods of the disclosure are used to quantify a transcription level (e.g., normalized gene expression value) of two or more TAAs, two or more neoantigens, two or more TSAs, or a combination thereof.

Certain cancers can be caused by, or correlate with, infections by a microorganism, such as but not limited to a virus, a bacterium, or a fungus. For example, certain strains of human papilloma virus are correlated with specific types of cervical cancer. Accordingly, in some embodiments, the one or more genes comprises a gene derived from a microorganism. In some embodiments, RNA is isolated from a biological sample disclosed herein. In some embodiments, RNA is isolated from microorganisms in a tumor. In some embodiments, RNA is isolated from microorganisms living on the skin, in the gastro-intestinal tract, in/on the reproductive organs, in the kidney and/or bladder, and/or in secretions from the above.

Specific genes and gene products can be associated with cancer. The RNA transcription level of one or more of these genes or a mutated form thereof associated with cancer can be quantified in a method of the present disclosure (e.g., via calculation of a normalized gene expression value). The one or more genes can comprise any gene(s) and/or mutated form(s) thereof that are associated with cancer, e.g., with cancer in general or with a specific type of cancer disclosed herein.

In some embodiments, one or more genes that are measured by a method of the disclosure and used to provide a wellness recommendation, provide a treatment recommendation, predict a response to a therapeutic agent or regimen, provide a diagnosis, provide a prognosis, provide an outcome prediction, identify a suitable therapeutic agent (e.g., drug, cancer vaccine, or checkpoint inhibitor), design a therapeutic agent (e.g., cancer vaccine, such as incorporation of an antigen from the gene in a cancer vaccine), identify a suitable combination therapy, identify a suitable clinical trial, and/or that are output into a report, comprise PARP1, PARP2, BRCA1, BRCA2, PD1, PDL1, CTLA4, CD86, DNMT1, YES1, ALK, FGFR3, VEGFA, BTK, HER2, CDK4, CDK6, ESR1, ESR2, PGR, AR, MKI67, TOP2A, TIM3, GITR, GITRL, ICOS, ICOSL, IDO1, LAG-3, NY-ESO-1, TERT, MAGEA3, TROP2, CEACAM5, RB1, P16, MRE11, RAD50, RAD51C, ATM, ATR, EMSY, NBS1, PALB2, PTEN, or a combination thereof.

In some embodiments, one or more genes that are measured by a method of the disclosure and used to provide a wellness recommendation, provide a treatment recommendation, predict a response to a therapeutic agent or regimen, provide a diagnosis, provide a prognosis, provide an outcome prediction, identify a suitable therapeutic agent (e.g., drug, cancer vaccine, or checkpoint inhibitor), design a therapeutic agent (e.g., cancer vaccine, such as incorporation of an antigen from the gene in a cancer vaccine), identify a suitable combination therapy, identify a suitable clinical trial, and/or that are output into a report, comprise PD1, PDL1, PDL2, CTLA4, TIM3, ICOS, IDO1, LAG3, GITR, CD273, LGALS9 TNRSF9, CD80, or CD86. In some embodiments, the one or more genes comprises a gene encoding a kinase gene product, e.g., CDK4, CDK6, CCND1, BTK, RET, EGFR, FGFR, BRAF, EGFR, FLT3, NTRK, KIT, MET, MEK, mTOR, RAF1, PKCA, JAK, BCR, ALK, PDGFR, PIK3CA. In some embodiments, the one or more genes comprises a gene encoding a product implicated in angiogenesis, e.g., VEGFA, FGF, FGFR, TGF-β, TNF-α, GMP. In some embodiments, the one or more genes comprises the gene encoding a gene product implicated in the mismatch repair pathway, e.g., hMLH1, hMSH2, hPMS1, hPMS2, or GTBP/hMSH6. In some embodiments, the one or more genes comprises the gene encoding a heat shock protein, e.g., HSP90B1. In some embodiments, the one or more genes comprises the gene encoding a calcium channel, e.g., TRPV6. In some embodiments, the one or more genes comprises the gene encoding a fusion gene coding for part of ALK, NTRK1, NTRK2, NTRK3, RET, ROS, ABL1, BCL2, or FGFR3. In some embodiments, the one or more genes comprises the gene encoding for genes involved in the homologous repair mechanism, e.g., BRCA1, BRCA2, PARP1, PARP2, PTEN, or RAD50. In some embodiments, the one or more genes comprises the gene encoding KRAS, RAS, or HRAS. In some embodiments, the one or more genes comprises the gene encoding Her2/ERBB2.

In some embodiments, one or more genes that are measured by a method of the disclosure and used to provide a wellness recommendation, provide a treatment recommendation, predict a response to a therapeutic agent or regimen, provide a diagnosis, provide a prognosis, provide an outcome prediction, identify a suitable therapeutic agent (e.g., drug, cancer vaccine, or checkpoint inhibitor), design a therapeutic agent (e.g., cancer vaccine, such as incorporation of an antigen from the gene in a cancer vaccine), identify a suitable combination therapy, identify a suitable clinical trial, and/or that are output into a report, comprise ABL1, ACP3, ADRB1, ALK, AR, AXL, BCL2, BCR, BCR-ABL, BRAF, BRCA1, BRCA2, BTK, CCR4, CD22, CD274, CD33, CD38, CD52, CD80, CDK4, CDK6, COX2, CRBN, CSF1R, CTLA4, CXCL8, CYP17A1, CYP19A1, DDR2, EGFR, EPHA2, ERBB2, ERBB4, ESR1, ESR2, ESR2, FER, FES, FGF2, FGFR, FGFR1, FGFR2, FGFR3, FGFR4, FKBP1A, FLT1, FLT3, FLT4, FRK, FYN, B4GALNT1, GNRHR, HDAC1, HDAC10, HDAC11, HDAC2, HDAC3, HDAC4, HDAC5, HDAC6, HDAC7, HDAC8, HDAC9, HER, IDH1, IDH2, IFNA1, IFNA2, IFNA5, IFNA6, IFNA8, IFNAR1, IFNAR2, IFNB1, IFNG, IGF1R, IL10, IL1A, IL2RA, IL2RB, IL2RG, IL3RA, IL6, JAK1, JAK2, KDR, KIT, KRAS, LCK, LHCGR, LTK, MAP2K1, MAP2K2, MAPK1, MAPK11, MET, MPL, MS4A1, MST1R, MTOR, NR3C1, NTRK1, NTRK2, NTRK3, PARP1, PARP2, PARP3, PDCD1, PDCD1, PDCD1LG2, PDGFRA, PDGFRB, PDGFRB, PGR, PIGF, PIK3CA, PIK3CD, PRKCA, PSMB1, PSMB10, PSMB2, PSMB5, PSMB8, PSMB8, PSMB9, PTGS2, PTK2, PTK2B, RAF1, RET, ROS1, SHH, SMO, SRC, SSTR1, SSTR2, SSTR3, SSTR4, SSTR5, STAT3, SYK, TEK, TLR7, TNF, TNF, TNFRSF8, TNFSF11, TNK2, VEGF, VEGFA, VEGFC, VEGFD, YES1, or any combination thereof.

In some embodiments, one or more genes that are measured by a method of the disclosure and used to provide a wellness recommendation, provide a treatment recommendation, predict a response to a therapeutic agent or regimen, provide a diagnosis, provide a prognosis, provide an outcome prediction, identify a suitable therapeutic agent (e.g., drug, cancer vaccine, or checkpoint inhibitor), design a therapeutic agent (e.g., cancer vaccine, such as incorporation of an antigen from the gene in a cancer vaccine), identify a suitable combination therapy, identify a suitable clinical trial, and/or that are output into a report, comprise ALK, AR, AURKA, B3GAT1, BAG1, BCL2, BCL6, BIRC5, CALB2, CALCA, CCNB1, CCND1, CD19, CD1A, CD2, CD200, CD247, CD274, CD28, CD3D, CD3E, CD3E, CD3G, CD4, CD5, CD52, CD68, CD7, CD8A, CDX2, CDX2, CEACAM5, CGA, CGB3, CHGA, CKBE, CLDN4, CR2, CTSV, CXCL13, DNTT, EPCAM, ERBB2, ERBB2, ESR1, ESR1, ESR1, FCER2, FCGR3A, FCGR3B, FUT4, GRB7, GSTM1, GZMB, GZMM, ICOS, IGK, IGL, IL2RA, INHA, KLK3, KRT20, KRT5, KRT6A, KRT6B, KRT7, LEF1, MK167, MK167, MLH1, MME, MMP11, MS4A1, MSH2, MSH6, MUC1, MUC16, MYBL2, NAPSA, NAPSA, NCAM1, NKX2-1, NKX2-1, NKX3-1, PAX2, PAX5, PAX8, PAX8, PDCD1, PDPN, PGR, PGR, PGR, PIP, PMS2, POU2AF1, POU2F2, PRF1, PTPRC, SATB2, SCUBE2, SELL, SYP, TCL1A, TG, TIA1, TNFRSF8, TP63, TP63, TRA, TRB, TRD, TRG, TSHB, WT1, or any combination thereof.

In some embodiments, one or more genes that are measured by a method of the disclosure and used to provide a wellness recommendation, provide a treatment recommendation, predict a response to a therapeutic agent or regimen, provide a diagnosis, provide a prognosis, provide an outcome prediction, identify a suitable therapeutic agent (e.g., drug, cancer vaccine, or checkpoint inhibitor), design a therapeutic agent (e.g., cancer vaccine, such as incorporation of an antigen from the gene in a cancer vaccine), identify a suitable combination therapy, identify a suitable clinical trial, and/or that are output into a report, comprise ACRBP, ACTL8, ADAM2, ADAM29, AKAP3, AKAP4, ANKRD45, ARMC3, ARX, ATAD2, BAGE, BAGE2, BAGE3, BAGE4, BAGE5, BRDT, C15orf60, C21orf99, CABYR, CAGE1, CALR3, CASC5, CCDC110, CCDC33, CCDC36, CCDC62, CCDC83, CDCA1, CEP290, CEP55, COX6B2, CPXCR1, CRISP2, CSAG1, CSAG2, CSAG3B, CT16.2, CT45A1, CT45A2, CT45A3, CT45A4, CT45A5, CT45A6, CT47A1, CT47A10, CT47A11, CT47A2, CT47A3, CT47A4, CT47A5, CT47A6, CT47A7, CT47A8, CT47A9, CT47B1, CT66/AA884595, CT69/BC040308, CT70/B1I818097, CTAG1A, CTAG1B, CTAG2, CTAGE-2, CTAGE1, CTAGE5, CTCFL, CTNNA2, CXorf48, Cxorf6l, cyclin A1, DCAF12, DDX43, DDX53, DKKL1, DMRT1, DNAJB8, DPPA2, DSCR8, EDAG, NDR, ELOVL4, FAM133A, FAM46D, FATE1, FBXO39, FMR1NB, FTHL17, GAGE1, GAGE12B, GAGE12C, GAGE12D, GAGE12E, GAGE12F, GAGE12G, GAGE12H, GAGE12I, GAGE12J, GAGE13, GAGE2A, GAGE3, GAGE4, GAGE5, GAGE6, GAGE7, GAGE8, GOLGAGL2 FA, GPAT2, GPATCH2, HIWI, MIWI, PIWI, HORMAD1, HORMAD2, HSPB9, IGSF11, IL13RA2, IMP-3, JARID1B, KIAA0100, LAGE-1b, LDHC, LEMD1, LIPI, LOC130576, LOC196993, LOC348120, LOC440934, LOC647107, LOC728137, LUZP4, LY6K, MAEL, MAGEA1, MAGEA10, MAGEA11, MAGEA12, MAGEA2, MAGEA2B, MAGEA3, MAGEA4, MAGEA5, MAGEA6, MAGEA8, MAGEA9, MAGEA9B/LOC728269, MAGEB1, MAGEB2, MAGEB3, MAGEB4, MAGEB5, MAGEB6, MAGEC1, MAGEC2, MAGEC3, MCAK, MMA1b, MORC1, MPHOSPH1, NLRP4, NOL4, NR6A1, NXF2, NXF2B, NY-ESO-1, ODF1, ODF2, ODF3, ODF4, OIP5, OTOA, PAGE1, PAGE2, PAGE2B, PAGE3, PAGE4, PAGE5, PASD1, PBK, PEPP2, PIWIL2, PLAC1, POTEA, POTEB, POTEC, POTED, POTEE, POTEG, POTEH, PRAME, PRM1, PRM2, PRSS54, PRSS55, PTPN20A, RBM46, RGS22, ROPN1, RQCD1, SAGE1, SEMG1, SLCO6A1, SPA17, SPACA3, SPAG1, SPAG17, SPAG4, SPAG6, SPAG8, SPAG9, SPANXA1, SPANXA2, SPANXB1, SPANXB2, SPANXC, SPANXD, SPANXE, SPANXN1, SPANXN2, SPANXN3, SPANXN4, SPANXN5, SPATA19, SPEF2, SPINLW1, SPO11, SSX1, SSX2, SSX2b, SSX3, SSX4, SSX4B, SSX5, SSX6, SSX7, SSX9, SYCE1, SYCP1, TAF7L, TAG, TDRD1, TDRD4, TDRD6, TEKT5, TEX101, TEX14, TEX15, TFDP3, THEG, TMEFF1, TMEFF2, TMEM108, TMPRSS12, TPPP2, TPTE, TSGA10, TSP50, TSPY1D, TSPY1E, TSPY1F, TSPY1G, TSPY1H, TSPY1I, TSPY2, TSPY3, TSSK6, TTK, TULP2, VENTXP1, XAGE-3b, XAGE-4/RP11-167P23.2, XAGE1, XAGE1B, XAGE1C, XAGE1D, XAGE1E, XAGE2, XAGE2B/CTD-2267G17.3, XAGE3, XAGE5, ZNF165, ZNF645, or any combination thereof.

In some embodiments, one or more genes that are measured by a method of the disclosure and used to provide a wellness recommendation, provide a treatment recommendation, predict a response to a therapeutic agent or regimen, provide a diagnosis, provide a prognosis, provide an outcome prediction, identify a suitable therapeutic agent (e.g., drug, cancer vaccine, or checkpoint inhibitor), design a therapeutic agent (e.g., cancer vaccine, such as incorporation of an antigen from the gene in a cancer vaccine), identify a suitable combination therapy, identify a suitable clinical trial, and/or that are output into a report, comprise A1CF, ABI1, ABL1, ABL2, ACKR3, ACSL3, ACSL6, ACVR1, ACVR2A, AFDN, AFF1, AFF3, AFF4, AKAP9, AKT1, AKT2, AKT3, ALDH2, ALK, AMER1, ANK1, APC, APOBEC3B, AR, ARAF, ARHGAP26, ARHGAP5, ARHGEF10, ARHGEF10L, ARHGEF12, ARID1A, ARID1B, ARID2, ARNT, ASPSCR1, ASXL1, ASXL2, ATF1, AT1C, ATM, ATP1A1, ATP2B3, ATR, ATRX, AXIN1, AXIN2, B2M, BAP1, BARD1, BAX, BAZ1A, BCL10, BCL11A, BCL11B, BCL2, BCL2L12, BCL3, BCL6, BCL7A, BCL9, BCL9L, BCLAF1, BCOR, BCORL1, BCR, BIRC3, BIRC6, BLM, BMP5, BMPR1A, BRAF, BRCA1, BRCA2, BRD3, BRD4, BRIP1, BTG1, BTK, BUB1B, C15orf65, CACNA1D, CALR, CAMTA1, CANT1, CARD11, CARS, CASP3, CASP8, CASP9, CBFA2T3, CBFB, CBL, CBLB, CBLC, CCDC6, CCNB1IP1, CCNC, CCND1, CCND2, CCND3, CCNE1, CCR4, CCR7, CD209, CD274, CD28, CD74, CD79A, CD79B, CDC73, CDH1, CDH10, CDH11, CDH17, CDK12, CDK4, CDK6, CDKN1A, CDKN1B, CDKN2A, CDKN2C, CDX2, CEBPA, CEP89, CHCHD7, CHD2, CHD4, CHEK2, CHIC2, CHST11, CIC, CIITA, CLIP1, CLP1, CLTC, CLTCL1, CNBD1, CNBP, CNOT3, CNTNAP2, CNTRL, COL1A1, COL2A1, COL3A1, COX6C, CPEB3, CREB1, CREB3L1, CREB3L2, CREBBP, CRLF2, CRNKL1, CRTC1, CRTC3, CSF1R, CSF3R, CSMD3, CTCF, CTNNA2, CTNNB1, CTNND1, CTNND2, CUL3, CUX1, CXCR4, CYLD, CYP2C8, CYSLTR2, DAXX, DCAF12L2, DCC, DCTN1, DDB2, DDIT3, DDR2, DDX10, DDX3X, DDX5, DDX6, DEK, DGCR8, DICER1, DNAJB1, DNM2, DNMT3A, DROSHA, DUX4L1, EBF1, ECT2L, EED, EGFR, EIF1AX, EIF3E, EIF4A2, ELF3, ELF4, ELK4, ELL, ELN, EML4, EP300, EPAS1, EPHA3, EPHA7, EPS15, ERBB2, ERBB3, ERBB4, ERC1, ERCC2, ERCC3, ERCC4, ERCC5, ERG, ESR1, ETNK1, ETV1, ETV4, ETV5, ETV6, EWSR1, EXT1, EXT2, EZH2, EZR, FAM131B, FAM135B, FAM47C, FANCA, FANCC, FANCD2, FANCE, FANCF, FANCG, FAS, FAT1, FAT3, FAT4, FBLN2, FBXO11, FBXW7, FCGR2B, FCRL4, FEN1, FES, FEV, FGFR1, FGFR1OP, FGFR2, FGFR3, FGFR4, FH, FHIT, FIPIL1, FKBP9, FLCN, FLI1, FLNA, FLT3, FLT4, FNBP1, FOXA1, FOXL2, FOXO1, FOXO3, FOXO4, FOXP1, FOXR1, FSTL3, FUBP1, FUS, GAS7, GATA1, GATA2, GATA3, GLI1, GMPS, GNA11, GNAQ, GNAS, GOLGA5, GOPC, GPC3, GPC5, GPHN, GRIN2A, GRM3, H3F3A, H3F3B, HERPUD1, HEY1, HIF1A, HIP1, HIST1H3B, HIST1H4I, HLA-A, HLF, HMGA1, HMGA2, HMGN2P46, HNF1A, HNRNPA2B1, HOOK3, HOXA11, HOXA13, HOXA9, HOXC11, HOXC13, HOXD11, HOXD13, HRAS, HSP90AA1, HSP90AB1, ID3, IDH1, IDH2, IGF2BP2, IGH, IGK, IGL, IKBKB, IKZF1, IL2, IL21R, IL6ST, IL7R, IRF4, IRS4, ISX, ITGAV, ITK, JAK1, JAK2, JAK3, JAZF1, JUN, KAT6A, KAT6B, KAT7, KCNJ5, KDM5A, KDM5C, KDM6A, KDR, KDSR, KEAP1, KIAA1549, KIF5B, KIT, KLF4, KLF6, KLK2, KMT2A, KMT2C, KMT2D, KNL1, KNSTRN, KRAS, KTN1, LARP4B, LASP1, LATS1, LATS2, LCK, LCP1, LEF1, LEPROTL1, LHFPL6, LIFR, LMNA, LMO1, LMO2, LPP, LRIG3, LRP1B, LSM14A, LYL1, LZTR1, MACC1, MAF, MAFB, MALAT1, MALT1, MAML2, MAP2K1, MAP2K2, MAP2K4, MAP3K1, MAP3K13, MAPK1, MAX, MB21D2, MDM2, MDM4, MDS2, MECOM, MED12, MEN1, MET, MGMT, MITF, MLF1, MLH1, MLLT1, MLLT10, MLLT11, MLLT3, MLLT6, MN1, MNX1, MPL, MRTFA, MSH2, MSH6, MSI2, MSN, MTCP1, MTOR, MUC1, MUC16, MUC4, MUTYH, MYB, MYC, MYCL, MYCN, MYD88, MYH11, MYH9, MYO5A, MYOD1, N4BP2, NAB2, NACA, NBEA, NBN, NCKIPSD, NCOA1, NCOA2, NCOA4, NCOR1, NCOR2, NDRG1, NF1, NF2, NFATC2, NFE2L2, NFIB, NFKB2, NFKBIE, NIN, NKX2-1, NONO, NOTCH1, NOTCH2, NPM1, NR4A3, NRAS, NRG1, NSD1, NSD2, NSD3, NT5C2, NTHL1, NTRK1, NTRK3, NUMA1, NUP214, NUP98, NUTM1, NUTM2B, NUTM2D, OLIG2, OMD, P2RY8, PABPC1, PAFAH1B2, PALB2, PATZ1, PAX3, PAX5, PAX7, PAX8, PBRM1, PBX1, PCBP1, PCM1, PDCD1LG2, PDE4DIP, PDGFB, PDGFRA, PDGFRB, PER1, PHF6, PHOX2B, PICALM, PIK3CA, PIK3CB, PIK3R1, PIM1, PLAG1, PLCG1, PML, PMS1, PMS2, POLD1, POLE, POLG, POLQ, POT1, POU2AF1, POU5F1, PPARG, PPFIBP1, PPM1D, PPP2R1A, PPP6C, PRCC, PRDM1, PRDM16, PRDM2, PREX2, PRF1, PRKACA, PRKAR1A, PRKCB, PRPF40B, PRRX1, PSIP1, PTCH1, PTEN, PTK6, PTPN11, PTPN13, PTPN6, PTPRB, PTPRC, PTPRD, PTPRK, PTPRT, PWWP2A, QKI, RABEP1, RAC1, RAD17, RAD21, RAD51B, RAF1, RALGDS, RANBP2, RAP1GDS1, RARA, RB1, RBM10, RBM15, RECQL4, REL, RET, RFWD3, RGPD3, RGS7, RHOA, RHOH, RMI2, RNF213, RNF43, ROBO2, ROS1, RPL10, RPL22, RPL5, RPN1, RSPO2, RSPO3, RUNX1, RUNX1T1, S100A7, SALL4, SBDS, SDC4, SDHA, SDHAF2, SDHB, SDHC, SDHD, 44444, 44445, 44448, SET, SETBP1, SETD1B, SETD2, SETDB1, SF3B1, SFPQ, SFRP4, SGK1, SH2B3, SH3GL1, SHTN1, SIRPA, SIX1, SIX2, SKI, SLC34A2, SLC45A3, SMAD2, SMAD3, SMAD4, SMARCA4, SMARCB1, SMARCD1, SMARCE1, SMC1A, SMO, SND1, SNX29, SOCS1, SOX2, SOX21, SPECC1, SPEN, SPOP, SRC, SRGAP3, SRSF2, SRSF3, SS18, SS18L1, SSX1, SSX2, SSX4, STAG1, STAG2, STAT3, STAT5B, STAT6, STIL, STK11, STRN, SUFU, SUZ12, SYK, TAF15, TAL1, TAL2, TBL1XR1, TBX3, TCEA1, TCF12, TCF3, TCF7L2, TCL1A, TEC, TENT5C, TERT, TET1, TET2, TFE3, TFEB, TFG, TFPT, TFRC, TGFBR2, THRAP3, TLX1, TLX3, TMEM127, TMPRSS2, TNC, TNFAIP3, TNFRSF14, TNFRSF17, TOP1, TP53, TP63, TPM3, TPM4, TPR. TRA, TRAF7, TRB, TRD, TRIM24, TRIM27, TRIM33, TRIP11, TRRAP, TSC1, TSC2, TSHR, U2AF1, UBR5, USP44, USP6, USP8, VAV1, VHL, VTI1A, WAS, WDCP, WIF1, WNK2, WRN, WT1, WWTR1, XPA, XPC, XPO1, YWHAE, ZBTB16, ZCCHC8, ZEB1, ZFHX3, ZMYM2, ZMYM3, ZNF331, ZNF384, ZNF429, ZNF479, ZNF521, ZNRF3, ZRSR2, or any combination thereof.

In some embodiments, the one or more genes comprise at least 5, at least 10, at least 20, at least 30, at least 50, at least 100, at least 200, at least 500, at least 1,000, or at least 5,000 genes. In some embodiments, the one or more genes comprise no more than 5,000 genes.

In some embodiments, the one or more genes comprise at most 5, at most 10, at most 20, at most 30, at most 50, at most 100, at most 200, at most 500, at most 1,000, at most 5,000 genes, or at most 10,000 genes. In some embodiments, the one or more genes comprise about 5, about 10, about 20, about 30, about 50, about 100, about 200, about 500, about 1,000, about 5,000 genes, or about 10,000 genes.

In some embodiments, a method of the disclosure comprises identification of a gene fusion. In some embodiments, a method of the disclosure comprises measuring an expression level (e.g., calculating a normalized gene expression value) of a gene fusion product. In some embodiments, a method of the disclosure comprises measuring an expression level (e.g., calculating a normalized gene expression value) of a gene that is commonly found in gene fusions, such as BCR, ABL1, ATIC, ALK, EML4, KLC1, NPM, SQSTM1, TFG, TPM3, TPM4, BCL2, FGFR3, NTRK1, NTRK2, NTRK3, ROS1, or REM. A gene fusion, gene fusion product, or gene commonly found in gene fusions can be a gene that is identified as aberrantly expressed as disclosed herein.

A gene fusion can be a hybrid gene formed from two previously independent genes. Gene fusion can occur as a consequence of e.g., translocation, interstitial deletion, or chromosomal inversion. Fusion genes have been found to be prevalent in many types of human neoplasia. The identification of these fusion genes can play important diagnostic and prognostic roles in methods of the disclosure. In some embodiments, a gene fusion can be identified by analysis of RNA sequencing reads that comprise sequences from both fusion components. In some embodiments, a gene fusion can be identified by aberrant expression (e.g., over-expression) of at least one of the previously independent genes. In some embodiments, data relating to gene fusions is output into a report disclosed herein for clinical decision making.

In some embodiments, a method of the disclosure is used to search for, identify, or measure expression of a BCR-ABL1, ATIC-ALK, EML4-ALK, KLC1-ALK, NPM-ALK, SQSTM1-ALK, TFG-ALK, TPM3-ALK, or TPM4-ALK gene fusion. In some embodiments, RNA sequencing of a BCR-ABL1, ATIC-ALK, EML4-ALK, KLC1-ALK, NPM-ALK, SQSTM1-ALK, TFG-ALK, TPM3-ALK, or TPM4-ALK gene fusion is used to identify a suitable therapeutic agent (e.g., drug, cancer vaccine, or checkpoint inhibitor), design a therapeutic agent (e.g., cancer vaccine, such as incorporation of an antigen from the gene in a cancer vaccine), used to identify a suitable combination therapy, or used to identify a suitable clinical trial. The suitable therapeutic agent can be any therapeutic agent disclosed herein. In some embodiments a fusion gene can both be a target for a treatment and a diagnostic at the same time, or it can be only one of the two.

In some embodiments, upon identification of a gene fusion, a report is generated that comprises a treatment recommendation regarding therapeutic use of nilotinib, dasatinib, bosutinib, ponatinib, imatinib, nilotinib, crizotinib, ceritinib, larotrectinib, selpercatinib (LOXO-292), BLU-667, or a combination thereof.

In some embodiments, methods of the disclosure can be used to predict the efficacy of a therapeutic agent, combination therapy, or treatment regimen. The predicted efficacy can be utilized in a wellness recommendation or clinical outcome predictor. Methods disclosed herein can produce normalized gene expression values that have a superior ability to integrate and compare gene expression data from diverse sources, which can result in improved ability to predict outcomes and identify associations compared to data processed by alternate methods. For example, in some embodiments, data from multiple sequencing runs, studies, clinical centers and databases can be combined and used in an algorithm disclosed herein to identify an association of a gene expression profile with clinical benefit upon treatment with a therapeutic agent.

In addition to identification of therapeutic agents (e.g., drugs) that are capable of targeting certain gene products, such as ER/tamoxifen described above, the present methods can identify new associations of clinical outcomes with a gene expression profile (e.g., a combination of normalized gene expression values and/or aberrantly expressed genes), therapeutic agents, and combinations thereof. The association can be an expected efficacy for a certain therapeutic agent, combination therapy, or treatment regimen based on the gene expression profile of the cancer. The association can be determined by an algorithm.

A clinical outcome predictor produced by a method or algorithm can be positive, i.e., a given therapeutic agent or treatment regimen is expected to provide a therapeutic benefit, or negative, i.e., a given therapeutic agent or treatment regimen is not expected to provide a therapeutic benefit.

Information beyond the gene expression data can be analyzed and can contribute to a wellness recommendation or clinical outcome predictor, for example, subject age, weight, sex. clinical history, disease stage, findings from other pathology tests, etc. The stage of cancer and the prognosis can be used to tailor a patient's therapy to provide a better outcome, e.g., systemic therapy and surgery, surgery alone, or systemic therapy alone. Risk assessment can be divided as desired, e.g., at the median, in tertiary groups, quaternary groups, and so on. Identification of pre-cancerous lesions can result in active surveillance using liquid biopsy methods or scanning (e.g. CAT or PET) and lifestyle interventions such as recommended changes to exercise regime and diet. In some embodiments, methods disclosed herein can be used to improve the efficacy of a chosen therapeutic agent or treatment regimen, e.g., by suggesting a candidate second therapeutic agent to use in combination with the chosen therapeutic agent.

An algorithm can be used to identify a combination of normalized gene expression values and/or aberrantly expressed genes) that are associated with high or low efficacy of a therapeutic agent or treatment regimen. The algorithm can utilize machine learning. The algorithm can be trained on input data that comprises, for example, normalized gene expression values and aberrantly expressed genes for subjects or biological samples, details of therapeutic agents or treatment regimens administered to each subject, subject age, weight, sex, clinical history, disease stage, findings from other pathology tests, disease staging, lymph node involvement, and outcome data, e.g., survival, average survival, five year survival rate, progression free survival, remission, relapse, minimal residual disease, disease stage progression, or a combination thereof.

The clinical outcome predictor can include calculating a disease prognostic algorithm utilizing outcome data or calculating a treatment response algorithm, e.g., where the treatment response algorithm is utilizing quantitative transcript data from checkpoint modulators and the corresponding ligand, tumor antigens or tumor-infiltrating immune cells, or any combination thereof. In some embodiments, a prognostic algorithm is developed using machine learning. In some embodiments, the predicting of clinical outcome provides a 5-year mortality risk assessment.

In some embodiments, an algorithm based on the measured gene expression levels is used to produce a prognostic value that can be utilized in a wellness recommendation or clinical outcome predictor. The algorithm can comprise as inputs normalized gene expression values determined by a method disclosed herein, genes identified as aberrantly expressed, and/or categorization of gene expression levels determined by a method disclosed herein. The algorithm can comprise as inputs, for example, clinical information such as lymph node involvement, age, other parameters, or a combination thereof.

The wellness recommendation can be, for example, a treatment recommendation. The treatment recommendation can be provided for an early stage cancer. The treatment recommendation can be provided for a late stage cancer. The treatment recommendation can include administering a therapeutic. The treatment recommendation can include not administering a therapeutic, e.g., because the tumor is classified as non-aggressive. The treatment recommendation can comprise not administering a therapeutic due to a lack of expected benefit.

In some embodiments, a method disclosed herein is used to detect recurrence and/or MRD (Minimal Residual Disease) of a cancer based on a gene expression profile of a test biological sample (e.g., normalized gene expression values and/or aberrantly expressed genes). The method can comprise comparing normalized gene expression values of the test biological sample to a plurality of control biological samples, for example, normal control sample, cancer control samples, relapsed/recurrent cancer control samples, or a combination thereof. Cancer-specific markers indicating recurrence can be detected. The method can optionally include providing a treatment recommendation.

In some embodiments a method of the disclosure identifies at least one target for a bespoke individualized treatment that is relevant and effective or potentially effective for the test subject from whom the test biological sample was obtained. In some embodiments a method identifies at least one target for a treatment that is relevant and effective in a wider context than the individual test subject from whom the test biological sample was obtained.

In some embodiments a method of the disclosure is used to identify more than one targets for a therapy, where at least one target is relevant and effective in a wider context than the individual test subject from whom the test biological sample (e.g., putative aberrant sample) is obtained and at least one target is only or mostly relevant and effective in the context of that one subject from whom the test biological sample is obtained. For example, the method can facilitate treatment with a combination of one or more general therapies and a bespoke individualized treatment.

In some embodiments, multiple gene expression comparisons can be connected using logical operations to produce composite gene expression indicators of some clinical parameter. For example, an indicator to predict whether a tumor is likely to respond to a treatment could be formulated as

Response=(AT<Q1_{AN}) OR (BT<Q3_{BN}) AND (CT>(Q3_{Cd}+1.5IQR_{CD}))
Where,
AT is the expression of gene A in the tumor;
BT is the expression of gene B in the tumor;
CT is the expression of gene C in the tumor;
Q1_{AN} is the expression of 1st quartile for gene A in the normal reference distribution;
Q3_{BN} is the expression of 3rd quartile for gene B in the normal reference distribution;
Q3_{CD} is the expression of 3rd quartile for gene C in the diseased reference distribution;
Q1_{CD} is the expression of 1st quartile for gene C in the diseased reference distribution; and
IQR_{CD} is the interquartile range for gene C in the diseased reference distribution, IQR_{CD}=Q3_{CD}−Q1_{CD}

The output of such an indicator can be binary, i.e., TRUE or FALSE; however, the gene expression states can be combined in other ways to produce a numeric output. For example, a prognostic indicator could be derived that computes the number of growth factor genes that are over-expressed in the tumor.

Predictors like those disclosed herein can be developed using empirical or model-based approaches, provided, for example, expression data are available for a statistically meaningful number of samples and relevant clinical data (such as drug response, diagnosis, survival, etc.) for each sample. Normal reference gene expression profiles and, optionally, diseased reference gene expression profiles can also be required. The genes used to compute the indicator, the method of setting thresholds used to define each gene state, and the logical relationships between states can all be included variables in the model.

Clinical significance can be assigned to the RNA transcription level of one or more genes based on a relationship to the control RNA transcription level for the one or more genes in a control tissue, e.g., a healthy tissue of the same type. In some embodiments, if a gene's expression level is tightly controlled (e.g., falls within a narrow range) in healthy tissues, then a relatively small deviation in expression can impact the physiological state of that tissue compared with genes whose levels fluctuate widely in normal tissue.

A method of treating a cancer in a test subject as described herein can comprise providing a computer-generated report that contains a recommendation for administering one or more therapeutic agents capable of effecting a change in RNA transcription level of one or more genes. Sequencing the RNA can occur from the 3′-end, the 5′-end, or a combination thereof, e.g., non-discriminately. The method can include: (a) quantifying a RNA transcription level of a gene in a test biological sample of the test subject comprising: (i) extracting RNA from the test biological sample from the test subject, (ii) measuring the RNA using an RNA sequencing kit comprising (1) sequencing the RNA from the 3′-end, and (2) identifying the RNA, (b) comparing the RNA transcription level of the gene in the test biological sample to a control RNA transcription level, and (c) treating the cancer in the test subject if the gene is identified as aberrantly expressed in the test biological sample relative to the control RNA transcription level. The treating can comprise administering a therapeutic agent capable of modulating the RNA transcription level of the gene, the amount of protein encoded by the gene, or the functional activity of the RNA and/or protein. The drug can be capable of directly or indirectly modifying the RNA transcription level, the protein translation level, or the functional activity of the one or more genes. For example, the drug can target the protein product encoded by the RNA. The drug can be any suitable therapeutic agent associated with an expression level of one or more genes. In some embodiments, treating the cancer comprises providing a report identifying a drug capable of modifying the RNA transcription level of the gene to the control RNA transcription level. In some embodiments, the gene is ER, PR, or ESR1 and the drug is tamoxifen. In some embodiments, the gene is PD-1 and the drug is nivolumab or ipilumimab.

Methods disclosed herein can comprise generating or outputting a report.

A report can comprise a quantitative gene expression value, such as a normalized gene expression value. A report can comprise two or more quantitative gene expression values, (e.g., normalized gene expression values). A report can comprise at least 1, at least 2, at least 3, at least 4, at least 5, at least 6, at least 7, at least 8, at least 9, at least 10, at least 15, at least 20, at least 25, at least 30, at least 40, at least 50, at least 100, at least 150, or at least 200 quantitative gene expression values, (e.g., normalized gene expression values). A report can comprise at most 1, at most 2, at most 3, at most 4, at most 5, at most 6, at most 7, at most 8, at most 9, at most 10, at most 15, at most 20, at most 25, at most 30, at most 40, at most 50, at most 100, at most 150, at most 200, at most 500, or at most 1,000 quantitative gene expression values, (e.g., normalized gene expression values). A report can comprise about 1, about 2, about 3, about 4, about 5, about 6, about 7, about 8, about 9, about 10, about 15, about 20, about 25, about 30, about 40, about 50, about 100, about 150, about 200, about 500, or about 1,000 quantitative gene expression values, (e.g., normalized gene expression values). One or more of the quantitative gene expression values, (e.g., normalized gene expression values) can be plotted, e.g., relative to a reference range, such as a distribution of expression of the gene in control biological samples.

A report can comprise a gene identified as aberrantly expressed, e.g., in a test biological sample relative to a plurality of control biological samples. A report can comprise two or more genes identified as aberrantly expressed. A report can comprise at least 1, at least 2, at least 3, at least 4, at least 5, at least 6, at least 7, at least 8, at least 9, at least 10, at least 15, at least 20, at least 25, at least 30, at least 40, at least 50, at least 100, at least 150, or at least 200 genes identified as aberrantly expressed. A report can comprise at most 1, at most 2, at most 3, at most 4, at most 5, at most 6, at most 7, at most 8, at most 9, at most 10, at most 15, at most 20, at most 25, at most 30, at most 40, at most 50, at most 100, at most 150, at most 200, at most 500, or at most 1,000 genes identified as aberrantly expressed. A report can comprise about 1, about 2, about 3, about 4, about 5, about 6, about 7, about 8, about 9, about 10, about 15, about 20, about 25, about 30, about 40, about 50, about 100, about 150, about 200, about 500, or about 1,000 genes identified as aberrantly expressed. One or more of the genes identified as aberrantly expressed can be plotted, e.g., relative to a reference range, such as a distribution of expression of the gene in control samples.

A report can comprise a wellness recommendation. A report can comprise two or more wellness recommendations. A report can comprise at least 1, at least 2, at least 3, at least 4, at least 5, at least 6, at least 7, at least 8, at least 9, at least 10, at least 15, at least 20, at least 25, at least 30, at least 40, at least 50, at least 100, at least 150, or at least 200 wellness recommendations. A report can comprise at most 1, at most 2, at most 3, at most 4, at most 5, at most 6, at most 7, at most 8, at most 9, at most 10, at most 15, at most 20, at most 25, at most 30, at most 40, at most 50, at most 100, at most 150, at most 200, at most 500, or at most 1,000 wellness recommendations. A report can comprise about 1, about 2, about 3, about 4, about 5, about 6, about 7, about 8, about 9, about 10, about 15, about 20, about 25, about 30, about 40, about 50, about 100, about 150, about 200, about 500, or about 1,000 wellness recommendations. The report can be or can comprise, for example, treatment recommendations disclosed herein.

A wellness recommendation (e.g., treatment recommendation) in the report can be based on categorization of expression (e.g., VERY LOW, LOW, NORMAL, HIGH, or VERY HIGH) and/or total/absolute expression counts of one or more genes.

A report can identify a therapeutic agent, combination therapy, treatment regimen, predicted response to a therapeutic agent or regimen, clinical trial, predicted outcome, or a combination thereof. A report can identify two or more therapeutic agents, combination therapies, treatment regimens, predicted responses to therapeutic agents or regimens, clinical trials, and/or predicted outcomes. A report can comprise at least 1, at least 2, at least 3, at least 4, at least 5, at least 6, at least 7, at least 8, at least 9, at least 10, at least 15, at least 20, at least 25, at least 30, at least 40, at least 50, at least 100, at least 150, or at least 200 therapeutic agents, combination therapies, treatment regimens, predicted responses to therapeutic agents or regimens, clinical trials, and/or predicted outcomes. A report can comprise at most 1, at most 2, at most 3, at most 4, at most 5, at most 6, at most 7, at most 8, at most 9, at most 10, at most 15, at most 20, at most 25, at most 30, at most 40, at most 50, at most 100, at most 150, at most 200, at most 500, or at most 1,000 therapeutic agents, combination therapies, treatment regimens, predicted responses to therapeutic agents or regimens, clinical trials, and/or predicted outcomes. A report can comprise about 1, about 2, about 3, about 4, about 5, about 6, about 7, about 8, about 9, about 10, about 15, about 20, about 25, about 30, about 40, about 50, about 100, about 150, about 200, about 500, or about 1,000 therapeutic agents, combination therapies, treatment regimens, predicted responses to therapeutic agents or regimens, clinical trials, and/or predicted outcomes.

A report can comprise groups of normalized gene expression values and/or aberrantly expressed genes. The normalized gene expression values and/or aberrantly expressed genes can be grouped based on biological function. The normalized gene expression values and/or aberrantly expressed genes can be grouped based on a class of therapeutic agent disclosed herein that targets the gene or that is indicated based on the expression level of the gene. Non-limiting examples of groups of genes that can be included in a report include homologous repair pathway genes, kinase target genes, immune checkpoint genes, hormone receptor genes, and fusion partners for drugs targeting gene fusions.

A report can be on physical media or can be stored (e.g., or displayed) on a computer.

In some embodiments, the report can be used to develop a therapeutic product, e.g., a cancer vaccine that includes one or more antigens identified as expressed (e.g., highly expressed) in the biological sample (e.g., cancer). In some embodiments, the report can be used to develop a diagnostic product or strategy, e.g., in cases when the one or more genes have not yet been known to correlate with a given disease, such as a cancer disclosed herein.

Methods of the disclosure can comprise providing a report identifying a therapeutic agent, e.g., a drug capable of modifying an RNA transcription level of the gene to the control RNA transcription level. The report can comprise any suitable therapeutic agent associated with an expression level of one or more genes. The report can comprise any suitable therapeutic agent(s) and/or genes. In some embodiments, the gene is ALK and the drug is crizotinib. In some embodiments, the gene is ER, PR, or ESR1 and the drug is tamoxifen. In some embodiments, the gene is PD-1 and the drug is nivolumab or ipilumimab. In some embodiments, the gene is HER2 and the drug is trastuzumab.

In some embodiments, a method of the disclosure comprises: (a) quantifying an RNA transcription level of a gene in test biological sample of a test subject comprising: (i) extracting RNA from the test biological sample from the test subject, (ii) measuring the RNA using an RNA sequencing kit comprising (1) sequencing the RNA from the 3′-end, and (2) identifying the RNA, (b) comparing the RNA transcription level of the gene to a control RNA transcription level, and (c) identifying a suitable therapeutic agent, regimen, or clinical trial if the gene is identified as aberrantly expressed in the test biological sample relative to the control RNA transcription level. In some embodiments, a report is generated that lists one or more genes identified as aberrantly expressed in the test biological sample. In some embodiments, a report is generated that lists one or more therapeutic agents, regimens, or clinical trials identified by the method.

Databases can be utilized in the methods disclosed herein.

A database can comprise gene expression counts, for example, of control biological samples, for normalization and/or for calling aberrantly expressed genes.

A database can comprise data identifying associations between gene expression data and therapeutic agents, treatment regiments, combination therapies, therapeutic efficacy, expected disease outcome, disease diagnosis, disease prognosis, and combinations thereof. A database can comprise data identifying associations between gene expression and efficacy of therapeutic agents.

A database can comprise data that can be used to identify associations (e.g., previously unknown associations) between gene expression data and therapeutic agents, treatment regiments, combination therapies, therapeutic efficacy, expected disease outcome, disease diagnosis, disease prognosis, and combinations thereof. A database can comprise data that can be used to identify associations between gene expression data and therapeutic efficacy.

A database can comprise, for example, normalized gene expression values (e.g., from subjects with disease or conditions, from normal control subjects, or a combination thereof), aberrantly expressed gene data (e.g., from subjects with disease or conditions, from normal control subjects, or a combination thereof). A database can comprise details of therapeutic agents. A database can comprise details of therapeutic regimens. A database can comprise clinical data, e.g., subject age. weight sex, clinical history, disease stage, findings from pathology tests, disease staging, and/or lymph node involvement. The clinical data can be associated with outcome data in the database, e.g., survival, average survival, five year survival rate, progression free survival, remission, relapse, minimal residual disease, disease stage progression, or a combination thereof.

One or more sources of medical information, including practice guidelines, clinical study reports, drug labels clinical trial records, and combinations thereof can be evaluated and the information therein used for generating the database. One or more sources of scientific information can be evaluated and the information therein used for generating the database. A database can comprise information from drug labels. A database can comprise information regarding treatment selection biomarkers from a drug label. A database can comprise information from drugbank. A database can comprise information from the NCI thesaurus.

In some embodiments, the disclosure provides one or more databases (e.g., custom-designed databases) that connect RNA transcription levels (e.g., normalized gene expression values) to relevant wellness recommendations, treatment recommendations, diagnoses, prognoses, therapeutic agents, combination therapies, treatment regimens, predicted responses to therapeutic agents or regimens, outcome predictions, and/or clinical trials.

A database can be used in methods of the disclosure, for example, for generation of a report that can support clinical decision making, e.g., by providing details of a therapeutic agent, regimen, combination therapy, or clinical trial that could be beneficial for a subject. The database can be used to generate a wellness recommendation, such as a treatment recommendation. In some embodiments, the report supports clinical decision making in a drug treatment regimen.

In some embodiments, a method disclosed herein is used to generate normalized gene expression values and/or identify aberrantly expressed genes, and the database is analyzed to provide a wellness recommendation, such as providing a treatment recommendation of administering a therapeutic agent or not administering a therapeutic agent.

Methods disclosed herein can support or comprise development of a treatment plan. Accordingly, the present method provides a system for determining a treatment plan for a patient diagnosed with a cancer, e.g., ovarian cancer or breast cancer, e.g., triple-negative breast cancer, comprising: (a) a processor; and (b) a database. A database entry can capture knowledge regarding how a given disease impacts or is associated with the expression of one or more genes, and how the detection of a change in gene expression can be used in clinical decision making. In some embodiments, a database record includes: (a) a unique identifier for one or more genes, (b) the corresponding gene expression state, e.g., the RNA expression level, that is associated with the diagnosis, prognosis, or clinical action (e.g., HIGH, LOW, VERY HIGH, VERY LOW, or NORMAL expression), (c) the patient biological sample type, (d) the biological sample type used to define the reference range, (e) the relevance of the gene expression state to at least one clinical decision, and (f) a reference to at least one reputable source of information to support the clinical annotation.

In an illustrative example, a database entry can comprise the gene identifier “ERBB2” (the HGNC gene symbol for the HER2-neu receptor) the gene expression state “over-expressed” “HIGH” or “VERY HIGH”, the disease cohort “metastatic gastric adenocarcinoma,” the sample type “gastric tumor,” the reference sample type “normal gastric tissue,” the clinical annotation “addition of trastuzumab to chemotherapy is recommended by clinical oncology practice guidelines,” and the reference: “NCCN Guidelines. Gastric Cancer (Version 3.2016). www.nccn.org/professionals/physician_gls/pdf/gastric.pdf. Accessed Mar. 20, 2017.” This database entry can be summarized in the following statement: “The NCCN guidelines recommends the addition of trastuzumab to chemotherapy for HER2-neu over-expressing metastatic adenocarcinomas.”

In another example, a database entry can comprise the gene identifier “NRG1” (the HGNC gene symbol for heregulin), the expression state “over-expressed” “HIGH” or “VERY HIGH”, the disease cohort “locally advanced or metastatic non-small cell lung cancer”, the patient sample type “NSCLC tumor,” the reference sample type “normal lung tissue,” the clinical action “eligibility for enrollment in a study to determine whether the combination of MM-121 plus docetaxel or pemetrexed is more effective than docetaxel or pemetrexed alone in regards to OS in patients with heregulin-positive NSCLC,” and the reference: “A Study of MM-121 in Combination With Chemotherapy Versus Chemotherapy Alone in Heregulin Positive NSCLC. (2015) Retrieved from clinicaltrials.gov/ct2 (Identification No. NCT02387216).”

In another example, a database entry can comprise the gene identifier “BRCA2”, the aberration type “under-expression” “LOW” or “VERY LOW”, the patient sample type “prostate tumor”, the reference sample type “normal prostate tissue”, the clinical relevance “In the TOPARP-A phase II trial, prostate cancer patients with loss of BRCA2 expression and other DNA repair defects exhibited a high rate of response to treatment with PARP inhibitor olaparib”, and the reference “Mateo J, Carreira S, Sandhu S, et al:DNA-repair defects and olaparib in metastatic prostate cancer. N Engl J Med 373:1697-1708, 2015.”

In some embodiments the database captures relevant medical and scientific knowledge for RNA transcription levels or protein expression levels of one or more genes quantified using methods disclosed herein. Scientifically and medically reputable sources of information can be used to link expression levels and changes to diagnoses, prognoses, and treatments, including peer reviewed medical journals, pharmaceutical drug labels, published clinical practice guidelines, and descriptions of registered clinical trials available through Clinicaltrials.gov and other public trial databases. In some embodiments, a clinical annotation is supported by one or more references, and any dissenting evidence can also be noted in the database.

A database can be assembled through manual curation, e.g., by persons with expertise in clinical medicine and/or genomics, by computer-automated text mining, or by combinations thereof. A database can be implemented as an SQL database, a NoSQL database program such as MongoDB, an Oracle database, a text file, or any other suitable of database formats.

### Cancers

In some embodiments, the methods of the present disclosure are useful for diagnosing or aiding in the treatment of a cancer having an RNA transcription level of one or more genes that is different compared with a control RNA transcription level from corresponding normal tissue. The methods can be used in relation to any cancer, including solid tumors and liquid cancers, e.g., leukemia or lymphoma. In some embodiments, the cancer is a solid tumor.

In some embodiments, the cancer comprises bladder cancer, brain cancer (e.g., astrocytoma, glioblastoma, meningioma, or oligodendroglioma), breast cancer (e.g., ER+, PR+, HER2+, or triple-negative breast cancer), bone cancer, cervical cancer, colon cancer, colorectal cancer, esophageal cancer, head and neck cancer, kidney cancer, liver cancer, lung cancer, medullary thyroid cancer, mouth cancer, nose cancer, ovarian cancer (e.g., mucinous, endometrioid, clear cell, or undifferentiated), pancreatic cancer, renal cancer, skin cancer, stomach cancer, throat cancer, thyroid cancer, or uterus cancer. In some embodiments, the cancer comprises bladder cancer, brain cancer, breast cancer, colon cancer, colorectal cancer, lung cancer, or ovarian cancer. In some embodiments, the cancer is lung cancer. In some embodiments, the cancer is brain cancer. In some embodiments, the cancer is breast cancer, e.g., triple-negative breast cancer. In some embodiments, the cancer is ovarian cancer. In some embodiments, the cancer is bladder cancer. In some embodiments, the cancer is colon cancer or colorectal cancer.

In some embodiments, the cancer is a carcinoma. In some embodiments, the cancer is a sarcoma. In some embodiments, the cancer is an adenoma.

In some embodiments, the cancer is of unknown primary tissue. In some embodiments, a method disclosed herein is used to identify the primary tissue type.

### Kits

Some embodiments provide a kit that can be used in any of the herein-described methods, e.g., materials that are used for RNA sequencing, and one or more additional components.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but a way to obtain the instructions from a remote source (e.g. via the Internet), can be provided. An example of this embodiment is a kit that includes a web address where the instructions can be viewed and/or from which the instructions can be downloaded. As with the instructions, this method for obtaining the instructions can be recorded on a suitable substrate.

### Computer Architectures and Systems

Methods disclosed herein can utilize computational devices. Methods disclosed herein can utilize a computer program product comprising a non-transitory computer-readable medium having computer-executable code encoded therein. The computer-executable code can be adapted to be executed to implement a method.

Computational devices disclosed herein can include any suitable combination of computing devices, including servers, interfaces, systems, databases, agents, peers, engines, controllers, modules, or other types of computing devices operating individually or collectively. Computing devices can comprise a processor configured to execute software instructions stored on a tangible, non-transitory computer readable storage medium (e.g., hard drive, field programmable gate array (FPGA), programmable logic array (PLA), solid state drive, RAM, flash, ROM, etc.). The software instructions can configure or otherwise program the computing device to provide the roles, responsibilities, or other functionality as discussed herein with respect to the disclosed apparatus. Disclosed technologies can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, databases, or interfaces exchange data using standardized protocols or algorithms, for example, based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

An aspect of the disclosure provides a system that is programmed or otherwise configured to implement the methods described herein. The system can include a computer server that is operatively coupled to an electronic device.

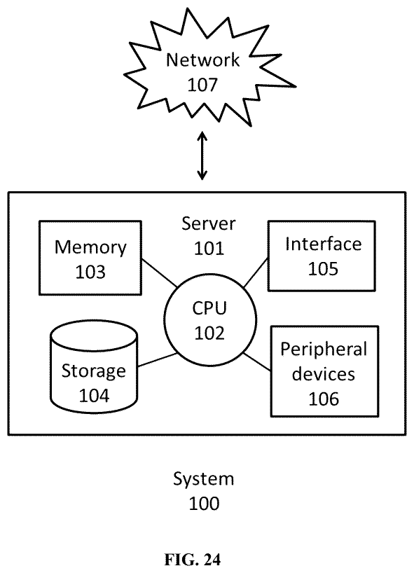

FIG. 24 illustrates a computer system 100 programmed or otherwise configured to allow implement methods disclosed herein. The system 100 includes a computer server (“server”) 101 that is programmed to implement methods disclosed herein. The server 101 includes a central processing unit (CPU) 102, which can be a single core or multi-core processor, or a plurality of processors for parallel processing. The server 101 also includes: a memory 103, such as random-access memory, read-only memory, and flash memory; electronic storage unit 104, such as a hard disk; communication interface 105, such as a network adapter, for communicating with one or more other systems; and peripheral devices 106, such as cache, other memory, data storage, and electronic display adapters. The memory 103, storage unit 104, interface 105, and peripheral devices 106 are in communication with the CPU 102 through a communication bus, such as a motherboard. The storage unit 104 can be a data storage unit or data repository for storing data. The server 101 can be operatively coupled to a computer network 107 with the aid of the communication interface 105. The network 107 can be the Internet, an internet or extranet, or an intranet or extranet that is in communication with the Internet. The network 107 in some cases is a telecommunications network or data network. The network 107 can include one or more computer servers, which can allow distributed computing, such as cloud computing. The network 107, in some cases with the aid of the server 101, can implement a peer-to-peer network, which can allow devices coupled to the server 101 to behave as a client or an independent server.

The storage unit 104 can store files, such as drivers, libraries, saved programs, files disclosed herein such as BCL files, FASTQ files, BAM files, SAM files, etc. The server 101, in some cases, can include one or more additional data storage units that are external to the server 101, such as located on a remote server that is in communication with the server 101 through an intranet or the Internet. The server 101 can communicate with one or more remote computer systems through the network 107.

In some embodiments, the system 100 includes a single server 101. In other situations, the system 100 includes multiple servers in communication with one another through an intranet or the Internet.

Methods as described herein can be implemented by way of a machine or computer executable code, modules, or software stored on an electronic storage location of the server 101, such as, for example, on the memory 103 or electronic storage unit 104. During use, the code can be executed by the processor 102. In some embodiments, the code can be retrieved from the storage unit 104 and stored on the memory 103 for ready access by the processor 102. In some embodiments, the electronic storage unit 104 can be precluded, and machine executable instructions are stored on memory 103. The code can be pre-compiled and configured for use with a processor adapted to execute the code, or can be compiled during runtime. The code can be supplied in a programming language that can be selected to allow the code to execute in a precompiled or as-compiled fashion.

All or portions of the software can at times be communicated through the Internet or various other telecommunications networks. Such communications can support loading of the software from one computer or processor into another, for example, from a management server or host computer into the computer platform of an application server. Another type of media that can bear the software elements includes optical, electrical, and electromagnetic waves, such as those used across physical interfaces between local devices, through wired and optical landline networks and over various air-links. The physical elements that carry such waves, such as wired or wireless links, or optical links, also can be considered as media bearing the software.

A machine readable medium, incorporating computer executable code, can take many forms, including a tangible storage medium, a carrier wave medium, and physical transmission medium. Non-limiting examples of non-volatile storage media include optical disks and magnetic disks, such as any of the storage devices in any computer. Volatile storage media include dynamic memory, such as a main memory of such a computer platform. Tangible transmission media include coaxial cables, copper wire, and fiber optics, including wires that comprise a bus within a computer system. Carrier wave transmission media can take the form of electric or electromagnetic signals, or acoustic or light waves such as those generated during radio frequency (RF) and infrared (IR) data communications.

Common forms of computer readable media include: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, and any other medium from which a computer can read programming code or data. Many of these forms of computer readable media can be involved in carrying one or more sequences of one or more instructions to a processor for execution.

The server 101 can be configured for: data mining; extract, transform, and load (ETL); or spidering operations, including Web Spidering. In Web Spidering, the system retrieves data from remote systems over a network and accesses an Application Programming Interface or parses the resulting markup. The process can permit the system to load information from a raw data source or mined data into a data warehouse.

Computer software can include computer programs, such as, for example executable files, libraries, and scripts. Software can include defined instructions that upon execution instruct computer hardware, for example, an electronic display to perform various tasks, such as display graphical elements on an electronic display. Software can be stored in computer memory.

Software can include machine executable code. Machine executable code can include machine language instructions specific to an individual computer processor, such as a CPU. Machine language can include groups of binary values signifying processor instructions that change the state of an electronic device, for example, a computer, from the preceding state. For example, an instruction can change the value stored in a particular storage location inside the computer. An instruction can also cause an output to be presented to a user, such as graphical elements to appear on an electronic display of a computer system. The processor can carry out the instructions in the order they are provided.

Software comprising one or more lines of code and output(s) therefrom can be presented to a user on a user interface (UI) of an electronic device of the user. Non-limiting examples of UIs include a graphical user interface (GUI) and web-based user interface. A GUI can allow a subject to access a display. The UI, such as GUI, can be provided on a display of an electronic device. Such displays can be used with other systems and methods of the disclosure.

Methods of the disclosure can be facilitated with the aid of applications, or apps, which can be installed on an electronic device of the user. An app can include a GUI on a display of the electronic device of the user. The app can be programmed or otherwise configured to perform various functions of the system. GUIs of apps can display on an electronic device. The electronic device can include, for example, a passive screen, a capacitive touch screen, or a resistive touch screen. The electronic device can include a network interface and a browser that allows that a user access various sites or locations, such as web sites, on an intranet or the Internet. The app is configured to allow the electronic device to communicate with a server, such as the server 101.

Any embodiment of the invention described herein can be, for example, produced and transmitted by a user within the same geographical location. Systems, products, or devices disclosed herein can be, for example, produced and/or transmitted from a geographic location in one country and a user of the invention can be present in a different country. In some embodiments, the data accessed by a system disclosed herein is a computer program product that can be transmitted from one of a plurality of geographic locations to a user. Data generated by a computer program product disclosed herein can be transmitted back and forth among a plurality of geographic locations, for example, by a network, a secure network, an insecure network, an internet, or an intranet. In some embodiments, data are encrypted. In some embodiments, a system herein is encoded on a physical and tangible product.

Further disclosed herein are computer systems that are programmed or otherwise configured to implement the methods described herein. Such computer systems can include a gene processing system having various components that execute the methods disclosed herein. Non-limiting examples of methods of the gene expression processing system include an expression count processing component; a gene identifying component; a recommendation component; an output component; and optionally a database of gene expression counts.

In some embodiments, a computer system includes a gene processing system comprises an expression count processing component; a gene identifying component; a recommendation component; an output component; a database of gene expression counts, or any combination thereof.

In some embodiments, a computer system includes a gene processing system comprises a database of gene expression counts, a subsampling component, a sorting component, a normalizing component, a deduplicating component, an output component, or any combination thereof,

### EMBODIMENTS

Embodiment 1. A method comprising: (a) processing gene expression counts of a test biological sample obtained from a test subject to obtain normalized gene expression values suitable for comparison to a database, wherein: the gene expression counts are generated by RNA sequencing of the test biological sample obtained from the test subject; the database comprises gene expression counts obtained from a plurality of control biological samples; and wherein each of the control biological samples is a sample type that is comparable to the test biological sample, and each of the control biological samples is independently obtained from a normal control subject; (b) identifying a gene that is aberrantly expressed in the test biological sample relative to the plurality of control biological samples; and (c) providing a wellness recommendation based on the gene that is aberrantly expressed in the test biological sample relative to the plurality of control biological samples.

Embodiment 2. The method of embodiment 1, further comprising identifying at least a second gene that is aberrantly expressed in the test biological sample relative to the plurality of control biological samples.

Embodiment 3. The method of embodiment 1 or embodiment 2, wherein the gene that is aberrantly expressed in the test biological sample relative to the plurality of control biological samples is a drug target.

Embodiment 4. The method of any one of embodiments 1-3, further comprising identifying a clinical trial in which the gene that is aberrantly expressed in the test biological sample relative to the plurality of control biological samples is a therapeutic target.

Embodiment 5. The method of any one of embodiments 1-4, wherein the gene that is aberrantly expressed in the test biological sample relative to the plurality of control biological samples encodes an immune modulatory protein.

Embodiment 6. The method of any one of embodiments 1-5, wherein the gene that is aberrantly expressed in the test biological sample relative to the plurality of control biological samples is an immune checkpoint gene.

Embodiment 7. The method of any one of embodiments 1-6, wherein the gene that is aberrantly expressed in the test biological sample relative to the plurality of control biological samples exhibits higher expression in the test biological sample than the plurality of control biological samples.

Embodiment 8. The method of any one of embodiments 1-7, wherein the gene that is aberrantly expressed in the test biological sample relative to the plurality of control biological samples exhibits lower expression in the test biological sample than the plurality of control biological samples.

Embodiment 9. The method of any one of embodiments 1-8, wherein a database containing a group of genes that are associated with treatment responses is used to determine whether the gene that is aberrantly expressed in the test biological sample relative to the plurality of control biological samples is associated with a treatment response for a disease.

10. The method of any one of embodiments 1-9, wherein the wellness recommendation comprises a treatment recommendation.

Embodiment 11. The method of any one of embodiments 1-10, further comprising generating a report, wherein the report identifies the gene that is aberrantly expressed in the test biological sample relative to the plurality of control biological samples.

Embodiment 12. The method of embodiment 11, wherein the report comprises the wellness recommendation.

Embodiment 13. The method of embodiment 11 or 12, wherein the report comprises quantitative gene expression values.

Embodiment 14. The method of any one of embodiments 1-13, wherein the wellness recommendation comprises a recommendation of administering a therapeutic agent to the test subject based on the gene that is aberrantly expressed in the test biological sample relative to the plurality of control biological samples.

Embodiment 15. The method of any one of embodiments 1-13, wherein the wellness recommendation comprises a recommendation of administering a therapeutic agent to the test subject based on an expression level of the gene that is aberrantly expressed in the test biological sample relative to the plurality of control biological samples.

Embodiment 16. The method of any one of embodiments 1-13, wherein the wellness recommendation comprises a recommendation of not administering a therapeutic agent to the test subject based on the gene that is aberrantly expressed in the test biological sample relative to the plurality of control biological samples.

Embodiment 17. The method of any one of embodiments 1-13, wherein the wellness recommendation comprises a recommendation of not administering a therapeutic agent to the test subject based on an expression level of the gene that is aberrantly expressed in the test biological sample relative to the plurality of control biological samples.

Embodiment 18. The method of any one of embodiments 1-17, further comprising identifying a therapeutic agent that modulates activity of the aberrantly expressed gene.

Embodiment 19. The method of any one of embodiments 1-18, further comprising identifying a therapeutic agent that modulates activity of a product encoded by the gene that is aberrantly expressed in the test biological sample relative to the plurality of control biological samples.

Embodiment 20. The method of any one of embodiments 1-19, wherein the gene that is aberrantly expressed in the test biological sample relative to the plurality of control biological samples is associated with an increased likelihood of a favorable response to a therapeutic agent.

Embodiment 21. The method of any one of embodiments 1-19, wherein the gene that is aberrantly expressed in the test biological sample relative to the plurality of control biological samples is associated with a reduced likelihood of a favorable response to a therapeutic agent.

Embodiment 22. The method of any one of embodiments 14-21, wherein the therapeutic agent comprises an immune checkpoint modulator.

Embodiment 23. The method of any one of embodiments 14-21, wherein the therapeutic agent comprises a kinase inhibitor.

Embodiment 24. The method of any one of embodiments 14-21, wherein the therapeutic agent comprises an anti-cancer chemotherapeutic.

Embodiment 25. The method of any one of embodiments 14-21, wherein the therapeutic agent comprises a cell therapy.

Embodiment 26. The method of any one of embodiments 14-21, wherein the therapeutic agent comprises a cancer vaccine.

Embodiment 27. The method of any one of embodiments 14-21, wherein the therapeutic agent comprises an mRNA vaccine.

Embodiment 28. The method of any one of embodiments 14-21, wherein the therapeutic agent comprises an RNA silencing (RNAi) agent.

Embodiment 29. The method of any one of embodiments 14-21, wherein the therapeutic agent comprises a gene editing agent.

Embodiment 30. The method of any one of embodiments 14-21, wherein the therapeutic agent comprises CRISPR/Cas system.

Embodiment 31. The method of any one of embodiments 14-21, wherein the therapeutic agent comprises an antibody.

Embodiment 32. The method of any one of embodiments 14-21, wherein the therapeutic agent comprises an RNA replacement therapy.

Embodiment 33. The method of any one of embodiments 14-21, wherein the therapeutic agent comprises a protein replacement therapy.

Embodiment 34. The method of any one of embodiments 1-33, further comprising making a diagnosis based on the gene that is aberrantly expressed in the test biological sample relative to the plurality of control biological samples.

Embodiment 35. The method of any one of embodiments 1-34, further comprising identifying a mutation in an expressed gene.

Embodiment 36. The method of any one of embodiments 1-35, wherein the database comprises gene expression counts obtained from at least 10 control biological samples.

Embodiment 37. The method of any one of embodiments 1-36, wherein the gene that is aberrantly expressed in the test biological sample relative to the plurality of control biological samples is identified by comparing the normalized gene expression values of the test biological sample to normalized gene expression values of the plurality of control biological samples.

Embodiment 38. The method embodiment 37, wherein the normalized gene expression values of the test biological sample and the normalized gene expression values of the plurality of control biological samples are normalized using a common normalization technique.

Embodiment 39. The method of embodiment 38, wherein the common normalization technique comprises quantile normalization.

Embodiment 40. The method of any one of embodiments 1-39, wherein the processing comprises subsampling the gene expression counts of the test biological sample obtained from the test subject, thereby generating subsampled gene expression counts from the test biological sample having a target number of assigned reads.

Embodiment 41. The method of embodiment 40, wherein the gene expression counts obtained from each control biological sample of the plurality are subsampled to the target number of assigned reads.

Embodiment 42. The method of any one of embodiments 1-41, wherein the identifying the gene that is aberrantly expressed in the test biological sample relative to the plurality of control biological samples comprises a non-parametric comparison of (i) a normalized gene expression value for a candidate gene from the test biological sample with (ii) a distribution of normalized gene expression values for the candidate gene obtained from the plurality of control biological samples.

Embodiment 43. The method of any one of embodiments 1-42, further comprising categorizing the normalized gene expression values of the test biological sample, wherein categories comprise VERY LOW, LOW, NORMAL, HIGH, and VERY HIGH categories, wherein: (i) the VERY HIGH category includes genes with a normalized gene expression value for the test biological sample that is greater than a threshold calculated based on distribution of a candidate gene's expression in the plurality of control biological samples and is lesser of: (i) a maximum normalized gene expression value for the candidate gene in the plurality of control biological samples; and (ii) a sum of third quartile (Q3) and 1.5 times interquartile range (IQR) of normalized gene expression values for the candidate gene in the plurality of control biological samples; (ii) the HIGH category includes genes not classified in the VERY HIGH category with a normalized gene expression value for the test biological sample that is greater than a sum of median plus two times IQR of the normalized gene expression values for the candidate gene in the plurality of control biological samples; (iii) the VERY LOW category includes genes with a normalized gene expression value for the test biological sample that is less than a threshold calculated based on distribution of the candidate gene's expression in the plurality of control biological samples and is lesser of: (i) minimum normalized gene expression value for the candidate gene in the plurality of control biological samples; and (ii) a difference of first quartile (Q1) and 1.5 times IQR of the normalized gene expression values for the candidate gene in the plurality of control biological samples; (iv) the LOW category includes genes not classified in the VERY LOW category with a normalized gene expression value for the test biological sample that is: (i) less than a difference of median and two times IQR of the normalized gene expression values for the candidate gene in the plurality of control biological samples; and (v) the NORMAL category is assigned to genes that are not categorized in the VERY LOW, LOW, HIGH, or VERY HIGH categories.

Embodiment 44. The method of any one of embodiments 1-42, further comprising categorizing the normalized gene expression values of the test biological sample, wherein categories comprise VERY LOW, LOW, NORMAL, HIGH, and VERY HIGH categories, wherein thresholds for the categories are calculated according to a non-parametric comparison of (a) a normalized gene expression value for a candidate gene in the test biological sample with (b) a distribution of normalized gene expression values for the candidate gene obtained from the plurality of control biological samples using equation 1, wherein: (i) yij represents expression of gene j in sample I; (ii) mediannj is a median expression level for gene j in the plurality of control biological samples; (iii) ynjmax is maximum expression of gene j in the plurality of control biological samples; (iv) ynjmin is minimum expression of gene j in the plurality of control biological samples; (v) Q1nj is a first quartile of gene j expression in the plurality of control biological samples; (vi) Q3nj is a third quartile of gene j expression in the plurality of control biological samples; (vii) IQRnj is an interquartile range of gene j expression in the plurality of control biological samples; and (viii) mj is a range of expression of gene j in the plurality of control biological samples and is calculated using equation 2, wherein equation 1 is:

$$f ( {y}_{ij} ) = \{ \begin{matrix}VERY HIGH , & if {y}_{ij} > {median}_{nj} + 2 * {r}_{nj} \\ HIGH , & if {y}_{ij} > min ( {y}_{{nj}_{max}} , {Q}_{{3}_{nj}} + 1.5 * {IQR}_{nj} ) \\ LOW , & if {y}_{ij} < max ( {y}_{{nj}_{min}} , {Q}_{{1}_{nj}} - 1.5 * {IQR}_{nj} ) \\ VERY LOW , & if {y}_{ij} < {median}_{nj} - 2 * {r}_{nj}\end{matrix}$$

wherein equation 2 is:

rₙⱼ=min(yₙⱼ_{ₘₐₓ},Q₃_{ₙⱼ}+1.5*IQRₙⱼ)−max(yₙⱼ_{ₘᵢₙ},Q₁_{ₙⱼ}−1.5*IQRₙⱼ)

Embodiment 45. The method of any one of embodiments 1-44, wherein the processing further comprises applying a scaling factor to the normalized gene expression values.

Embodiment 46. The method embodiment 45, wherein the scaling factor is calculated using a third quartile (Q3) value of the normalized gene expression values of the test biological sample.

Embodiment 47. The method of embodiment 46, wherein the normalized gene expression values are divided by the scaling factor, multiplied by a scalar, and log transformed.

Embodiment 48. The method of embodiment 46, wherein the normalized gene expression values are divided by the scaling factor, multiplied by 1,000, and log 2 transformed.

Embodiment 49. The method of any one of embodiments 1-48, wherein the test biological sample comprises tumor tissue.

Embodiment 50. The method of any one of embodiments 1-49, wherein the test biological sample comprises cancer cells.

Embodiment 51. The method of any one of embodiments 1-50, wherein the test biological sample is formalin-fixed and paraffin-embedded (FFPE).

Embodiment 52. The method of any one of embodiments 1-50, wherein the test biological sample is a fresh frozen sample.

Embodiment 53. The method of any one of embodiments 1-48, wherein the test biological sample is a saliva sample.

Embodiment 54. The method of any one of embodiments 1-50, wherein the test biological sample is a blood sample.

Embodiment 55. The method of any one of embodiments 1-48, wherein the test biological sample is a urine sample.

Embodiment 56. The method of any one of embodiments 1-55, wherein RNA extracted from the test biological sample has a DV200 value of less than about 30%.

Embodiment 57. The method of any one of embodiments 1-56, wherein the test subject has a disease.

Embodiment 58. The method of any one of embodiments 1-56, wherein the test subject is suspected of having a disease.

Embodiment 59. The method of any one of embodiments 57-58, wherein the disease is a cancer.

Embodiment 60. The method of any one of embodiments 57-58, wherein the disease is breast cancer.

Embodiment 61. The method of any one of embodiments 58-60, wherein the gene that is aberrantly expressed in the test biological sample relative to the plurality of control biological samples is identified without analyzing gene expression counts obtained from a biological sample of a second subject that has the disease.

Embodiment 62. The method of any one of embodiments 1-61, wherein the gene that is aberrantly expressed in the test biological sample relative to the plurality of control biological samples is identified without analyzing gene expression counts obtained from a second biological sample from a control tissue of the test subject.

Embodiment 63. The method of any one of embodiments 1-62, wherein the gene that is aberrantly expressed in the test biological sample relative to the plurality of control biological samples is identified without analyzing gene expression values obtained from a matched normal or adjacent normal biological sample from the test subject.

Embodiment 64. The method of any one of embodiments 1-63, wherein the test biological sample and each of the control biological samples comprise tissue samples of a same tissue type.

Embodiment 65. The method of any one of embodiments 1-63, wherein the test subject has a cancer that has metastasized to a metastatic site, wherein each of the control biological samples is of a same tissue type as a tissue type in the metastatic site.

Embodiment 66. The method of any one of embodiments 1-65, wherein the plurality of control biological samples are obtained from subjects that are matched to the test subject based on age.

Embodiment 67. The method of any one of embodiments 1-66, wherein the plurality of control biological samples are obtained from subjects that are matched to the test subject based on sex.

Embodiment 68. The method of any one of embodiments 1-67, wherein identifying the gene that is aberrantly expressed in the test biological sample relative to the plurality of control biological samples does not include comparing gene expression counts or normalized gene expression values from (i) a first cohort comprising the test subject and at least two additional subjects to (ii) a second cohort comprising at least three subjects.

Embodiment 69. The method of any one of embodiments 1-68, wherein the test subject is not part of a cohort study.

Embodiment 70. The method of any one of embodiments 1-69, wherein RNA extracted from the test biological sample is subjected to de-crosslinking at about 80° C. for at least 11 minutes.

Embodiment 71. The method of any one of embodiments 1-70, wherein the processing further comprises removing duplicate reads identified as originating from a same RNA molecule.

Embodiment 72. The method of any one of embodiments 1-70, wherein the processing further comprises removing duplicate reads identified as originating from a same RNA molecule based on a unique molecular identifier (UMI) appended to each RNA molecule.

Embodiment 73. The method of any one of embodiments 1-72, wherein the RNA sequencing of the test biological sample comprises dual indexing.

Embodiment 74. The method of any one of embodiments 1-73, wherein the RNA sequencing of the test biological sample comprises adding unique molecular identifiers (UMIs) and dual indexes to cDNA molecules.

Embodiment 75. The method of any one of embodiments 1-74, wherein the RNA sequencing of the test biological sample comprises 3′ end sequencing.

Embodiment 76. The method of any one of embodiments 1-75, wherein the RNA sequencing of the test biological sample comprises poly(T) priming.

Embodiment 77. The method of any one of embodiments 1-76, wherein the normalized gene expression values comprise data for mRNAs.

Embodiment 78. The method of any one of embodiments 1-77, wherein the normalized gene expression values comprise data for non-coding RNAs.

Embodiment 79. The method of any one of embodiments 1-78, wherein the normalized gene expression values comprise data for miRNAs.

Embodiment 80. The method of any one of embodiments 1-79, wherein the gene that is aberrantly expressed in the test biological sample relative to the plurality of control biological samples is suitable for inclusion in a cancer vaccine.

Embodiment 81. The method of embodiment 80, further comprising identifying at least a second gene that is aberrantly expressed in the test biological sample relative to the plurality of control biological samples that is suitable for inclusion in the cancer vaccine.

Embodiment 82. The method of any one of embodiments 1-81, wherein the gene that is aberrantly expressed in the test biological sample relative to the plurality of control biological samples is included in a cancer vaccine.

Embodiment 83. The method of any one of embodiments 1-81, wherein the gene that is aberrantly expressed in the test biological sample relative to the plurality of control biological samples is included in a cancer vaccine and a second gene that is aberrantly expressed in the test biological sample relative to the plurality of control biological samples is included in the cancer vaccine.

Embodiment 84. The method of any one of embodiments 1-83, wherein the gene that is aberrantly expressed in the test biological sample relative to the plurality of control biological samples comprises a tumor associated antigen.

Embodiment 85. The method of any one of embodiments 1-84, wherein the gene that is aberrantly expressed in the test biological sample relative to the plurality of control biological samples comprises a neoepitope.

Embodiment 86. The method of any one of embodiments 1-85, further comprising developing a therapeutic targeting the aberrantly expressed gene.

Embodiment 87. The method of any one of embodiments 1-86, further comprising developing a therapeutic targeting a product encoded by the aberrantly expressed gene.

Embodiment 88. A method comprising processing gene expression counts of a test biological sample to obtain normalized gene expression values suitable for comparison to a database, wherein the database comprises gene expression counts from a plurality of control biological samples, wherein: (a) the gene expression counts of the test biological sample are: (i) generated by RNA sequencing of the test biological sample; (ii) subsampled to a target number of assigned reads; and (iii) sorted by a total of gene expression counts assigned to each gene, thereby generating sorted gene expression counts of the test biological sample; (b) the gene expression counts of each control biological sample of the plurality are: (i) generated by RNA sequencing of the control biological sample; (ii) subsampled to the target number of assigned reads; and (iii) sorted by a total of gene expression counts assigned to each gene, thereby generating sorted gene expression counts of the control biological sample; and (c) the processing comprises, for each position of the sorted gene expression counts of the test biological sample, calculating a normalized gene expression value from an average of: (i) gene expression count at the position of the sorted gene expression counts of the test biological sample; and (ii) gene expression count for each of the plurality of control biological samples at a corresponding position of the sorted gene expression counts of the control biological sample; thereby generating the normalized gene expression values suitable for comparison to the database.

Embodiment 89. The method of embodiment 88, wherein the processing further comprises removing duplicate reads identified as originating from a same RNA molecule.

Embodiment 90. The method embodiment 88, wherein the processing further comprises removing duplicate reads identified as originating from a same RNA molecule based on a unique molecular identifier (UMI) appended to each RNA molecule.

Embodiment 91. The method of any one of embodiments 88-90, wherein the processing comprises quantile normalization.

Embodiment 92. The method of any one of embodiments 88-91, wherein the non-zero total gene expression counts assigned to each gene of the test biological sample are sorted from lowest count to highest count.

Embodiment 93. The method of any one of embodiments 88-91, wherein the non-zero total gene expression counts assigned to each gene of the test biological sample are sorted from highest count to lowest count.

Embodiment 94. The method of any one of embodiments 88-93, wherein the database comprises gene expression counts obtained from at least 10 control biological samples.

Embodiment 95. The method of any one of embodiments 88-94, wherein the database comprises normalized control gene expression values of each control biological sample of the plurality, wherein the normalized control gene expression values are calculated by a technique that comprises quantile normalization.

Embodiment 96. The method of any one of embodiments 88, wherein the normalized gene expression values of the test biological sample and normalized gene expression values from the plurality of control biological samples are normalized using a common normalization technique.

Embodiment 97. The method of any one of embodiments 88-96, wherein the normalization technique does not include analysis of spike-in controls.

Embodiment 98. The method of any one of embodiments 88-97, further comprising categorizing the normalized gene expression values of the test biological sample, wherein categories comprise VERY LOW, LOW, NORMAL, HIGH, and VERY HIGH categories, wherein: i. the VERY HIGH category includes genes with a normalized gene expression value for the test biological sample that is greater than a threshold calculated based on distribution of a candidate gene's expression in the plurality of control biological samples and is lesser of: (i) a maximum normalized gene expression value for the candidate gene in the plurality of control biological samples; and (ii) a sum of Q3 and 1.5 times IQR of normalized gene expression values for the candidate gene in the plurality of control biological samples; ii. the HIGH category includes genes not classified in the VERY HIGH category with a normalized gene expression value for the test biological sample that is greater than a sum of median plus two times IQR of the normalized gene expression values for the candidate gene in the plurality of control biological samples; iii. the VERY LOW category includes genes with a normalized gene expression value for the test biological sample that is less than a threshold calculated based on distribution of a candidate gene's expression in the plurality of control biological samples and is lesser of: (i) minimum normalized gene expression value for the candidate gene in the plurality of control biological samples; and (ii) a difference of Q1 and 1.5 times IQR of the normalized gene expression values for the candidate gene in the plurality of control biological samples; iv. the LOW category includes genes not classified in the VERY LOW category with a normalized gene expression value for the test biological sample that is: (i) less than a difference of median and two times IQR of the normalized gene expression values for the candidate gene in the plurality of control biological samples; and v. the NORMAL category is assigned to genes that are not categorized in the VERY LOW, LOW, HIGH, or VERY HIGH categories.

Embodiment 99. The method of any one of embodiments 88-97, further comprising categorizing the normalized gene expression values of the test biological sample, wherein categories comprise VERY LOW, LOW, NORMAL, HIGH, and VERY HIGH categories, wherein thresholds for the categories are calculated according to a non-parametric comparison of (a) a normalized gene expression value for a candidate gene in the test biological sample with (b) a distribution of normalized gene expression values for the candidate gene obtained from the plurality of control biological samples using equation 1, wherein: (i) yij represents expression of gene j in sample I; (ii) mediannj is a median expression level for gene j in the plurality of control biological samples; (iii) ynjmax is maximum expression of gene j in the plurality of control biological samples; (iv) ynjmin is minimum expression of gene j in the plurality of control biological samples; (v) Q1nj is a first quartile of gene j expression in the plurality of control biological samples; (vi) Q3nj is a third quartile of gene j expression in the plurality of control biological samples; (vii) IQRnj is an interquartile range of gene j expression in the plurality of control biological samples; and (viii) mj is a range of expression of gene j in the plurality of control biological samples and is calculated using equation 2; wherein equation 1 is:

$$f ( {y}_{ij} ) = \{ \begin{matrix}VERY HIGH , & if {y}_{ij} > {median}_{nj} + 2 * {r}_{nj} \\ HIGH , & if {y}_{ij} > min ( {y}_{{nj}_{max}} , {Q}_{{3}_{nj}} + 1.5 * {IQR}_{nj} ) \\ LOW , & if {y}_{ij} < max ( {y}_{{nj}_{min}} , {Q}_{{1}_{nj}} - 1.5 * {IQR}_{nj} ) \\ VERY LOW , & if {y}_{ij} < {median}_{nj} - 2 * {r}_{nj}\end{matrix}$$

wherein equation 2 is:

rₙⱼ=min(yₙⱼ_{ₘₐₓ},Q₃_{ₙⱼ}+1.5IQRₙⱼ)−max(yₙⱼ_{ₘᵢₙ},Q₁_{ₙⱼ}−1.5*IQRₙⱼ).

Embodiment 100. The method of any one of embodiments 88-100, wherein the processing further comprises applying a scaling factor to the normalized gene expression values

Embodiment 101. The method of embodiment 100, wherein the scaling factor is calculated using a third quartile (Q3) value of the normalized gene expression values of the test biological sample.

Embodiment 102. The method of any one of embodiments 101-101, wherein the normalized gene expression values are divided by the scaling factor, multiplied by a scalar, and log transformed.

Embodiment 103. The method of any one of embodiments 101-101, wherein the normalized gene expression values are divided by the scaling factor, multiplied by 1,000, and log 2 transformed.

Embodiment 104. The method of any one of embodiments 88-103, further comprising identifying a gene that is aberrantly expressed in the test biological sample relative to the plurality of control biological samples.

Embodiment 105. The method of embodiment 104, further comprising identifying at least a second gene that is aberrantly expressed in the test biological sample relative to the plurality of control biological samples.

Embodiment 106. The method embodiment 104 or embodiment 105, wherein the identifying the gene that is aberrantly expressed in the test biological sample relative to the plurality of control biological samples comprises a non-parametric comparison of (i) a normalized gene expression value for a candidate gene from the test biological sample with (ii) a distribution of normalized gene expression values for the candidate gene obtained from the plurality of control biological samples.

Embodiment 107. The method of any one of embodiments 104-106, wherein the gene that is aberrantly expressed in the test biological sample relative to the plurality of control biological samples is a drug target.

Embodiment 108. The method of any one of embodiments 104-107, further comprising identifying a clinical trial in which the gene that is aberrantly expressed in the test biological sample relative to the plurality of control biological samples is a therapeutic target.

Embodiment 109. The method of any one of embodiments 104-108, wherein the gene that is aberrantly expressed in the test biological sample relative to the plurality of control biological samples encodes an immune modulatory protein.

Embodiment 110. The method of any one of embodiments 104-109, wherein the gene that is aberrantly expressed in the test biological sample relative to the plurality of control biological samples is an immune checkpoint gene.

Embodiment 111. The method of any one of embodiments 104-110, wherein the gene that is aberrantly expressed in the test biological sample relative to the plurality of control biological samples exhibits higher expression in the test biological sample than the plurality of control biological samples.

Embodiment 112. The method of any one of embodiments 104-110, wherein the gene that is aberrantly expressed in the test biological sample relative to the plurality of control biological samples exhibits lower expression in the test biological sample than the plurality of control biological samples.

Embodiment 113. The method of any one of embodiments 104-112, wherein a database containing a group of genes that are associated with treatment responses is used to determine whether the gene that is aberrantly expressed in the test biological sample relative to the plurality of control biological samples is associated with a treatment response for a disease.

Embodiment 114. The method of any one of embodiments 88-113, further comprising providing a wellness recommendation.

Embodiment 115. The method of embodiment 114, wherein the wellness recommendation comprises a treatment recommendation.

Embodiment 116. The method of any one of embodiments 104-113, further comprising generating a report, wherein the report identifies the gene that is aberrantly expressed in the test biological sample relative to the plurality of control biological samples.

Embodiment 117. The method of embodiment 116, wherein the report comprises a wellness recommendation.

Embodiment 118. The method of any one of embodiments 116-117, wherein the report comprises quantitative gene expression values.

Embodiment 119. The method of any one of embodiments 114-115 and 117-118, wherein the test biological sample is from a subject, wherein the wellness recommendation comprises a recommendation of administering a therapeutic agent to the subject based on the gene that is aberrantly expressed in the test biological sample relative to the plurality of control biological samples.

Embodiment 120. The method of any one of embodiments 114-115 and 117-119, wherein the test biological sample is from a subject, wherein the wellness recommendation comprises a recommendation of administering a therapeutic agent to the subject based on an expression level of the gene that is aberrantly expressed in the test biological sample relative to the plurality of control biological samples.

Embodiment 121. The method of any one of embodiments 114-115 and 117-120, wherein the test biological sample is from a subject, wherein the wellness recommendation comprises a recommendation of not administering a therapeutic agent to the subject based on the gene that is aberrantly expressed in the test biological sample relative to the plurality of control biological samples.

Embodiment 122. The method of any one of embodiments 114-115 and 117-120, wherein the test biological sample is from a subject, wherein the wellness recommendation comprises a recommendation of not administering a therapeutic agent to the subject based on an expression level of the gene that is aberrantly expressed in the test biological sample relative to the plurality of control biological samples.

Embodiment 123. The method of any one of embodiments 104-122, further comprising identifying a therapeutic agent that modulates activity of the aberrantly expressed gene.

Embodiment 124. The method of any one of embodiments 104-123, further comprising identifying a therapeutic agent that modulates activity of a product encoded by the gene that is aberrantly expressed in the test biological sample relative to the plurality of control biological samples.

Embodiment 125. The method of any one of embodiments 104-124, wherein the gene that is aberrantly expressed in the test biological sample relative to the plurality of control biological samples is associated with an increased likelihood of a favorable response to a therapeutic agent.

Embodiment 126. The method of any one of embodiments 104-124, wherein the gene that is aberrantly expressed in the test biological sample relative to the plurality of control biological samples is associated with a reduced likelihood of a favorable response to a therapeutic agent.

Embodiment 127. The method of any one of embodiments 119-126, wherein the therapeutic agent comprises an immune checkpoint modulator.

Embodiment 128. The method of any one of embodiments 119-126, wherein the therapeutic agent comprises a kinase inhibitor.

Embodiment 129. The method of any one of embodiments 119-126, wherein the therapeutic agent comprises an anti-cancer chemotherapeutic.

Embodiment 130. The method of any one of embodiments 119-126, wherein the therapeutic agent comprises a cell therapy.

Embodiment 131. The method of any one of embodiments 119-126, wherein the therapeutic agent comprises a cancer vaccine.

Embodiment 132. The method of any one of embodiments 119-126, wherein the therapeutic agent comprises an mRNA vaccine.

Embodiment 133. The method of any one of embodiments 119-126, wherein the therapeutic agent comprises an RNA silencing (RNAi) agent.

Embodiment 134. The method of any one of embodiments 119-126, wherein the therapeutic agent comprises a gene editing agent.

Embodiment 135. The method of any one of embodiments 119-126, wherein the therapeutic agent comprises CRISPR/Cas system.

Embodiment 136. The method of any one of embodiments 119-126, wherein the therapeutic agent comprises an antibody.

Embodiment 137. The method of any one of embodiments 119-126, wherein the therapeutic agent comprises an RNA replacement therapy.

Embodiment 138. The method of any one of embodiments 119-126, wherein the therapeutic agent comprises a protein replacement therapy.

Embodiment 139. The method of any one of embodiments 104-138, further comprising making a diagnosis based on the gene that is aberrantly expressed in the test biological sample relative to the plurality of control biological samples.

Embodiment 140. The method of any one of embodiments 88-139, further comprising identifying a mutation in an expressed gene.

Embodiment 141. The method of any one of embodiments 88-140, wherein the test biological sample comprises tumor tissue.

Embodiment 142. The method of any one of embodiments 88-141, wherein the test biological sample comprises cancer cells.

Embodiment 143. The method of any one of embodiments 88-142, wherein the test biological sample is formalin-fixed and paraffin-embedded (FFPE).

Embodiment 144. The method of any one of embodiments 88-142, wherein the test biological sample is a fresh frozen sample.

Embodiment 145. The method of any one of embodiments 88-140, wherein the test biological sample is a saliva sample.

Embodiment 146. The method of any one of embodiments 88-142, wherein the test biological sample is a blood sample.

Embodiment 147. The method of any one of embodiments 88-140, wherein the test biological sample is a urine sample.

Embodiment 148. The method of any one of embodiments 88-147, wherein RNA extracted from the test biological sample has a DV200 value of less than about 30%.

Embodiment 149. The method of any one of embodiments 119-148, wherein the subject has a disease.

Embodiment 150. The method of any one of embodiments 119-148, wherein the subject is suspected of having a disease.

Embodiment 151. The method of any one of embodiments 149-150, wherein the disease is a cancer.

Embodiment 152. The method of any one of embodiments 149-150, wherein the disease is breast cancer.

Embodiment 153. The method of any one of embodiments 104-148, wherein the test biological sample is from a first subject that has a disease, wherein the gene that is aberrantly expressed in the test biological sample relative to the plurality of control biological samples is identified without analyzing gene expression counts obtained from a biological sample of a second subject that has or is suspected of having the disease.

Embodiment 154. The method of any one of embodiments 104-148, wherein the test biological sample is from a subject that has a disease, wherein the gene that is aberrantly expressed in the test biological sample relative to the plurality of control biological samples is identified without analyzing gene expression values obtained from a second biological sample from a control tissue of the subject.

Embodiment 155. The method of any one of embodiments 104-148, wherein the test biological sample is from a first subject that has a cancer, wherein the gene that is aberrantly expressed in the test biological sample relative to the plurality of control biological samples is identified without analyzing gene expression values obtained from a matched normal or adjacent normal biological sample from the subject.

Embodiment 156. The method of any one of embodiments 88-155, wherein the test biological sample and each of the control biological samples comprise tissue samples of a same tissue type.

Embodiment 157. The method of any one of embodiments 88-155, wherein the test biological sample is from a subject, wherein the subject has a cancer that has metastasized to a metastatic site, wherein each of the control biological samples is of a same tissue type as a tissue type in the metastatic site.

Embodiment 158. The method of any one of embodiments 88-157, wherein the test biological sample is from a test subject, wherein the plurality of control biological samples are obtained from subjects that are matched to the test subject based on age.

Embodiment 159. The method of any one of embodiments 88-157, wherein the test biological sample is from a test subject, wherein the plurality of control biological samples are obtained from subjects that are matched to the test subject based on sex.

Embodiment 160. The method of any one of embodiments 88-157, wherein the test biological sample is from a test subject, wherein the plurality of control biological samples are obtained from subjects that are matched to the test subject based on disease.

Embodiment 161. The method of any one of embodiments 104-156, wherein the test biological sample is from a first subject, wherein identifying the gene that is aberrantly expressed in the test biological sample relative to the plurality of control biological samples does not include comparing gene expression counts or normalized gene expression values from (i) a first cohort comprising the first subject and at least two additional subjects to (ii) a second cohort comprising at least three control subjects.

Embodiment 162. The method of any one of embodiments 88-156, wherein the test biological sample is from a subject, wherein the subject is not part of a cohort study.

Embodiment 163. The method of any one of embodiments 88-162, wherein RNA extracted from the test biological sample is subjected to de-crosslinking at about 80° C. for at least 11 minutes.

Embodiment 164. The method of any one of embodiments 88-163, wherein the RNA sequencing of the test biological sample comprises dual indexing.

Embodiment 165. The method of any one of embodiments 88-164, wherein the RNA sequencing of the test biological sample comprises adding unique molecular identifiers (UMIs) and dual indexes to cDNA molecules.

Embodiment 166. The method of any one of embodiments 88-165, wherein the RNA sequencing of the test biological sample comprises 3′ end sequencing.

Embodiment 167. The method of any one of embodiments 88-166, wherein the RNA sequencing of the test biological sample comprises poly(T) priming.

Embodiment 168. The method of any one of embodiments 88-167, wherein the normalized gene expression values comprise data for mRNAs.

Embodiment 169. The method of any one of embodiments 88-168, wherein the normalized gene expression values comprise data for non-coding RNAs.

Embodiment 170. The method of any one of embodiments 88-169, wherein the normalized gene expression values comprise data for miRNAs.

Embodiment 171. The method of any one of embodiments 104-170, wherein the gene that is aberrantly expressed in the test biological sample relative to the plurality of control biological samples is suitable for inclusion in a cancer vaccine.

Embodiment 172. The method of embodiment 171, further comprising identifying at least a second gene that is aberrantly expressed in the test biological sample relative to the plurality of control biological samples that is suitable for inclusion in the cancer vaccine.

Embodiment 173. The method of any one of embodiments 104-170, wherein the gene that is aberrantly expressed in the test biological sample relative to the plurality of control biological samples is included in a cancer vaccine.

Embodiment 174. The method of any one of embodiments 104-170, wherein the gene that is aberrantly expressed in the test biological sample relative to the plurality of control biological samples is included in a cancer vaccine and a second gene that is aberrantly expressed in the test biological sample relative to the plurality of control biological samples is included in the cancer vaccine.

Embodiment 175. The method of any one of embodiments 104-174, wherein the gene that is aberrantly expressed in the test biological sample relative to the plurality of control biological samples comprises a tumor associated antigen.

Embodiment 176. The method of any one of embodiments 104-175, wherein the gene that is aberrantly expressed in the test biological sample relative to the plurality of control biological samples comprises a neoepitope.

Embodiment 177. The method of any one of embodiments 104-176, further comprising developing a therapeutic targeting the gene that is aberrantly expressed in the test biological sample relative to the plurality of control biological samples.

Embodiment 178. The method of any one of embodiments 104-177, further comprising developing a therapeutic targeting a product encoded by the gene that is aberrantly expressed in the test biological sample relative to the plurality of control biological samples.

Embodiment 179. A computer program product comprising a non-transitory computer-readable medium having computer-executable code encoded therein, the computer-executable code adapted to be executed to implement a method, the method comprising: a) running a gene processing system, wherein the gene processing system comprises: i) an expression count processing component; ii) a gene identifying component; iii) a recommendation component; iv) a database of gene expression counts obtained from a plurality of control biological samples, wherein each of the control biological samples is a sample type that is comparable to a test biological sample, and each of the control biological samples is independently obtained from a normal control subject; and v) an output component; b) processing, by the expression count processing component, gene expression counts of RNA sequencing of the test biological sample obtained from a test subject to obtain gene expression values suitable for comparison to the database; c) identifying, by the gene identifying component, a gene that is aberrantly expressed in the test biological sample relative to the plurality of control biological samples; d) providing a wellness recommendation, by the recommendation component, based on the gene that is aberrantly expressed in the test biological sample relative to the plurality of control biological samples; and e) outputting, by the output component, a report that comprises the wellness recommendation.

Embodiment 180. The computer program product of embodiment 179, wherein the method further comprises identifying, by the gene identifying component, at least a second gene that is aberrantly expressed in the test biological sample relative to the plurality of control biological samples.

Embodiment 181. The computer program product of any one of embodiments 179-180, wherein the gene that is aberrantly expressed in the test biological sample relative to the plurality of control biological samples is a drug target.

Embodiment 182. The computer program product of any one of embodiments 179-181, wherein the gene that is aberrantly expressed in the test biological sample relative to the plurality of control biological samples encodes an immune modulatory protein.

Embodiment 183. The computer program product of any one of embodiments 179-182, wherein the gene that is aberrantly expressed in the test biological sample relative to the plurality of control biological samples is an immune checkpoint gene.

Embodiment 184. The computer program product of any one of embodiments 179-183, wherein providing the wellness recommendation, by the recommendation component, comprises using a database containing a group of genes that are associated with treatment responses to determine whether the gene that is aberrantly expressed in the test biological sample relative to the plurality of control biological samples is associated with a treatment response for a disease.

Embodiment 185. The computer program product of any one of embodiments 179-184, wherein the wellness recommendation comprises a treatment recommendation.

Embodiment 186. The computer program product of any one of embodiments 179-185, wherein the report identifies the gene that is aberrantly expressed in the test biological sample relative to the plurality of control biological samples.

Embodiment 187. The computer program product of any one of embodiments 179-186, wherein the report comprises quantitative gene expression values.

Embodiment 188. The computer program product of any one of embodiments 179-187, wherein the wellness recommendation comprises a recommendation of administering a therapeutic agent to the test subject based on the gene that is aberrantly expressed in the test biological sample relative to the plurality of control biological samples.

Embodiment 189. The computer program product of any one of embodiments 179-187, wherein the wellness recommendation comprises a recommendation of administering a therapeutic agent to the test subject based on an expression level of the gene that is aberrantly expressed in the test biological sample relative to the plurality of control biological samples.

Embodiment 190. The computer program product of any one of embodiments 179-187, wherein the wellness recommendation comprises a recommendation of not administering a therapeutic agent to the test subject based on the gene that is aberrantly expressed in the test biological sample relative to the plurality of control biological samples.

Embodiment 191. The computer program product of any one of embodiments 179-187, wherein the wellness recommendation comprises a recommendation of not administering a therapeutic agent to the test subject based on an expression level of the gene that is aberrantly expressed in the test biological sample relative to the plurality of control biological samples.

Embodiment 192. The computer program product of any one of embodiments 179-191, wherein the method further comprises identifying, by the recommendation component, a therapeutic agent that modulates activity of the gene that is aberrantly expressed in the test biological sample relative to the plurality of control biological samples.

Embodiment 193. The computer program product of any one of embodiments 179-192, wherein the method further comprises identifying, by the recommendation component, a therapeutic agent that modulates activity of a product encoded by the gene that is aberrantly expressed in the test biological sample relative to the plurality of control biological samples.

Embodiment 194. The computer program product of any one of embodiments 188-193, wherein the therapeutic agent comprises an immune checkpoint modulator.

Embodiment 195. The computer program product of any one of embodiments 188-193, wherein the therapeutic agent comprises a kinase inhibitor.

Embodiment 196. The computer program product of any one of embodiments 188-193, wherein the therapeutic agent comprises an anti-cancer chemotherapeutic.

Embodiment 197. The computer program product of any one of embodiments 188-193, wherein the therapeutic agent comprises a cell therapy.

Embodiment 198. The computer program product of any one of embodiments 188-193, wherein the therapeutic agent comprises a cancer vaccine.

Embodiment 199. The computer program product of any one of embodiments 188-193, wherein the therapeutic agent comprises an mRNA vaccine.

Embodiment 200. The computer program product of any one of embodiments 188-193, wherein the therapeutic agent comprises an RNA silencing (RNAi) agent.

Embodiment 201. The computer program product of any one of embodiments 188-193, wherein the therapeutic agent comprises a gene editing agent.

Embodiment 202. The computer program product of any one of embodiments 188-193, wherein the therapeutic agent comprises CRISPR/Cas system.

Embodiment 203. The computer program product of any one of embodiments 188-193, wherein the therapeutic agent comprises an antibody.

Embodiment 204. The computer program product of any one of embodiments 188-193, wherein the therapeutic agent comprises an RNA replacement therapy.

Embodiment 205. The computer program product of any one of embodiments 188-193, wherein the therapeutic agent comprises a protein replacement therapy.

Embodiment 206. The computer program product of any one of embodiments 179-205, wherein the database comprises gene expression counts obtained from at least 10 control biological samples.

Embodiment 207. The computer program product of any one of embodiments 179-206, wherein the identifying, by the identifying component, comprises comparing the gene expression values of the test biological sample to gene expression values of the plurality of control biological samples.

Embodiment 208. The computer program product of embodiment 207, wherein the gene expression values of the test biological sample and the gene expression values of the plurality of control biological samples are normalized using a common normalization technique.

Embodiment 209. The computer program product of embodiment 208, wherein the common normalization technique comprises quantile normalization.

Embodiment 210. The computer program product of any one of embodiments 179-209, wherein the processing, by the expression count processing component, comprises subsampling the gene expression counts of the test biological sample obtained from the test subject, thereby generating subsampled gene expression counts from the test biological sample having a target number of assigned reads.

Embodiment 211. The computer program product of embodiment 210, wherein the gene expression counts obtained from each control biological sample of the plurality are subsampled to the target number of assigned reads.

Embodiment 212. The computer program product of any one of embodiments 179-211, wherein the identifying, by the gene identifying component, the gene that is aberrantly expressed in the test biological sample relative to the plurality of control biological samples comprises a non-parametric comparison of (i) a normalized gene expression value for a candidate gene from the test biological sample with (ii) a distribution of normalized gene expression values for the candidate gene obtained from the plurality of control biological samples.

Embodiment 213. The computer program product of any one of embodiments 179-212, wherein the method further comprises categorizing, by the gene identifying component, the gene expression values of the test biological sample, wherein categories comprise VERY LOW, LOW, NORMAL, HIGH, and VERY HIGH categories, wherein: i. the VERY HIGH category includes genes with a gene expression value for the test biological sample that is greater than a threshold calculated based on distribution of a candidate gene's expression in the plurality of control biological samples and is lesser of: (i) a maximum gene expression value for the candidate gene in the plurality of control biological samples; and (ii) a sum of Q3 and 1.5 times IQR of gene expression values for the candidate gene in the plurality of control biological samples; ii. the HIGH category includes genes not classified in the VERY HIGH category with a gene expression value for the test biological sample that is greater than a sum of median plus two times IQR of the gene expression values for the candidate gene in the plurality of control biological samples; iii. the VERY LOW category includes genes with a gene expression value for the test biological sample that is less than a threshold calculated based on distribution of the candidate gene's expression in the plurality of control biological samples and is lesser of: (i) minimum gene expression value for the candidate gene in the plurality of control biological samples; and (ii) a difference of Q1 and 1.5 times IQR of the gene expression values for the candidate gene in the plurality of control biological samples; iv. the LOW category includes genes not classified in the VERY LOW category with a gene expression value for the test biological sample that is: (i) less than a difference of median and two times IQR of the gene expression values for the candidate gene in the plurality of control biological samples; and v. the NORMAL category is assigned to genes that are not categorized in the VERY LOW, LOW, HIGH, or VERY HIGH categories.

Embodiment 214. The computer program product of any one of embodiments 179, wherein the method further comprises categorizing, by the gene identifying component, the gene expression values of the test biological sample, wherein categories comprise VERY LOW, LOW, NORMAL, HIGH, and VERY HIGH categories, wherein thresholds for the categories are calculated according to a non-parametric comparison of (a) a gene expression value for a candidate gene in the test biological sample with (b) a distribution of gene expression values for the candidate gene obtained from the plurality of control biological samples using equation 1, wherein: (i) yij represents expression of gene j in sample I; (ii) mediannj is a median expression level for gene j in the plurality of control biological samples; (iii) ynjmax is maximum expression of gene j in the plurality of control biological samples; (iv) ynjmin is minimum expression of gene j in the plurality of control biological samples; (v) Q1nj is a first quartile of gene j expression in the plurality of control biological samples; (vi) Q3nj is a third quartile of gene j expression in the plurality of control biological samples; (vii) IQRnj is an interquartile range of gene j expression in the plurality of control biological samples; and (viii) mj is a range of expression of gene j in the plurality of control biological samples and is calculated using equation 2; wherein equation 1 is:

$$f ( {y}_{ij} ) = \{ \begin{matrix}VERY HIGH , & if {y}_{ij} > {median}_{nj} + 2 * {r}_{nj} \\ HIGH , & if {y}_{ij} > min ( {y}_{{nj}_{max}} , {Q}_{{3}_{nj}} + 1.5 * {IQR}_{nj} ) \\ LOW , & if {y}_{ij} < max ( {y}_{{nj}_{min}} , {Q}_{{1}_{nj}} - 1.5 * {IQR}_{nj} ) \\ VERY LOW , & if {y}_{ij} < {median}_{nj} - 2 * {r}_{nj}\end{matrix}$$

wherein equation 2 is:

rₙⱼ=min(yₙⱼ_{ₘₐₓ},Q₃_{ₙⱼ}+1.5*IQRₙⱼ)−max(yₙⱼ_{ₘᵢₙ},Q₁_{ₙⱼ}−1.5*IQRₙⱼ).

Embodiment 215. The computer program product of any one of embodiments 179-214, wherein the processing, by the expression count processing component, further comprises applying a scaling factor to the gene expression values.

Embodiment 216. The computer program product of embodiment 215, wherein the scaling factor is calculated using a third quartile (Q3) value of the normalized gene expression values of the test biological sample.

Embodiment 217. The method of embodiment 216, wherein the normalized gene expression values are divided by the scaling factor, multiplied by a scalar, and log transformed.

Embodiment 218. The method of embodiment 216, wherein the normalized gene expression values are divided by the scaling factor, multiplied by 1,000, and log 2 transformed

Embodiment 219. The computer program product of any one of embodiments 179-218, wherein the test subject has a disease.

Embodiment 220. The computer program product of any one of embodiments 179-219, wherein the test subject is suspected of having a disease.

Embodiment 221. The computer program product of any one of embodiments 219-220, wherein the disease is a cancer.

Embodiment 222. The computer program product of any one of embodiments 219-220, wherein the disease is breast cancer.

Embodiment 223. The computer program product of any one of embodiments 179-222, wherein identifying, by the gene identifying component, the gene that is aberrantly expressed in the test biological sample relative to the plurality of control biological samples does not include comparing gene expression counts or normalized gene expression values from (i) a first cohort comprising the test subject and at least two additional subjects to (ii) a second cohort comprising at least three control subjects.

Embodiment 224. The computer program product of any one of embodiments 179-223, wherein the processing, by the expression count processing component, further comprises removing duplicate reads identified as originating from a same RNA molecule.

Embodiment 225. The computer program product of any one of embodiments 179-223, wherein the processing, by the expression count processing component, further comprises removing duplicate reads identified as originating from a same RNA molecule based on a unique molecular identifier (UMI) appended to each RNA molecule.

Embodiment 226. The computer program product of any one of embodiments 179-225, wherein the gene expression values comprise data for mRNAs.

Embodiment 227. The computer program product of any one of embodiments 179-226, wherein the gene expression values comprise data for non-coding RNAs.

Embodiment 228. The computer program product of any one of embodiments 179-227, wherein the gene expression values comprise data for miRNAs.

Embodiment 229. The computer program product of any one of embodiments 179-228, wherein the gene that is aberrantly expressed in the test biological sample relative to the plurality of control biological samples comprises a tumor associated antigen.

Embodiment 230. The computer program product of any one of embodiments 179-229, wherein the gene that is aberrantly expressed in the test biological sample relative to the plurality of control biological samples comprises a neoepitiope.

Embodiment 231. A computer program product comprising a non-transitory computer-readable medium having computer-executable code encoded therein, the computer-executable code adapted to be executed to implement a method, the method comprising: a) running a gene processing system, wherein the gene processing system comprises: i) a database of gene expression counts obtained from a plurality of control biological samples; ii) a subsampling component; iii) a sorting component; iv) a normalizing component; and v) an output component; b) subsampling, by the subsampling component, gene expression counts of RNA sequencing of a test biological sample obtained from a test subject to a target number of assigned reads, thereby generating subsampled gene expression counts of the test biological sample; c) sorting, by the sorting component, a total of gene expression counts of the subsampled gene expression counts of the test biological sample to obtain sorted gene expression counts of the test biological sample; d) subsampling, by the subsampling component, gene expression counts of RNA sequencing of each control biological sample of the plurality to the target number of assigned reads, thereby generating subsampled gene expression counts of each of the control biological samples; e) sorting, by the sorting component, a total of gene expression counts of the subsampled gene expression counts of each of the control biological samples to obtain sorted gene expression counts of each of the control biological samples; f) normalizing, by the normalizing component, the sorted gene expression counts of the test biological sample to obtain normalized gene expression values of the test biological sample, wherein the normalizing comprises, for each position of the sorted gene expression counts of the test biological sample, calculating a normalized gene expression value from an average of: (i) gene expression count at the position of the sorted gene expression counts of the test biological sample; and (ii) gene expression count for each of the plurality of control biological samples at a corresponding position of the sorted gene expression counts of the control biological sample; and g) outputting, by the output component, the normalized gene expression values of the test biological sample.

Embodiment 232. The computer program product of embodiment 231, wherein the gene processing system further comprises a gene identifying component, wherein the method further comprises identifying, by the gene identifying component, a gene that is aberrantly expressed in the test biological sample relative to the plurality of control biological samples.

Embodiment 233. The computer program product of embodiment 232, wherein the method further comprises identifying, by the gene identifying component, at least a second gene that is aberrantly expressed in the test biological sample relative to the plurality of control biological samples, wherein the gene and the second gene are different.

Embodiment 234. The computer program product of any one of embodiments 232-233, wherein the gene that is aberrantly expressed in the test biological sample relative to the plurality of control biological samples is a drug target.

Embodiment 235. The computer program product of any one of embodiments 232-234, wherein the gene that is aberrantly expressed in the test biological sample relative to the plurality of control biological samples encodes an immune modulatory protein.

Embodiment 236. The computer program product of any one of embodiments 232-235, wherein the gene that is aberrantly expressed in the test biological sample relative to the plurality of control biological samples is an immune checkpoint gene.

Embodiment 237. The computer program product of any one of embodiments 232-236, wherein the gene processing system further comprises a recommendation component, wherein the method further comprises providing a wellness recommendation, by the recommendation component, based on the gene that is aberrantly expressed in the test biological sample relative to the plurality of control biological samples.

Embodiment 238. The computer program product of embodiment 237, wherein the providing the wellness recommendation, by the recommendation component, comprises using a database containing a group of genes that are associated with treatment responses to determine whether the gene that is aberrantly expressed in the test biological sample relative to the plurality of control biological samples is associated with a treatment response for a disease.

Embodiment 239. The computer program product of any one of embodiments 237-238, wherein the wellness recommendation comprises a treatment recommendation.

Embodiment 240. The computer program product of any one of embodiments 232-239, wherein the method further comprises outputting, by the output component, a report identifying the gene that is aberrantly expressed in the test biological sample relative to the plurality of control biological samples.

Embodiment 241. The computer program product of embodiment 240, wherein the report comprises quantitative gene expression values.

Embodiment 242. The computer program product of any one of embodiments 237-241, wherein the method further comprises outputting, by the output component, a report comprising the wellness recommendation based on the gene that is aberrantly expressed in the test biological sample relative to the plurality of control biological samples.

Embodiment 243. The computer program product of any one of embodiments 237-242, wherein the wellness recommendation comprises a recommendation of administering a therapeutic agent to the test subject based on the gene that is aberrantly expressed in the test biological sample relative to the plurality of control biological samples.

Embodiment 244. The computer program product of any one of embodiments 237-242, wherein the wellness recommendation comprises a recommendation of administering a therapeutic agent to the test subject based on an expression level of the gene that is aberrantly expressed in the test biological sample relative to the plurality of control biological samples.

Embodiment 245. The computer program product of any one of embodiments 237-242, wherein the wellness recommendation comprises a recommendation of not administering a therapeutic agent to the test subject based on the gene that is aberrantly expressed in the test biological sample relative to the plurality of control biological samples.

Embodiment 246. The computer program product of any one of embodiments 237-242, wherein the wellness recommendation comprises a recommendation of not administering a therapeutic agent to the test subject based on an expression level of the gene that is aberrantly expressed in the test biological sample relative to the plurality of control biological samples.

Embodiment 247. The computer program product of any one of embodiments 237-246, wherein the method further comprises identifying, by the recommendation component, a therapeutic agent that modulates activity of the gene that is aberrantly expressed in the test biological sample relative to the plurality of control biological samples.

Embodiment 248. The computer program product of any one of embodiments 237-247, wherein the method further comprises identifying, by the recommendation component, a therapeutic agent that modulates activity of a product encoded by the gene that is aberrantly expressed in the test biological sample relative to the plurality of control biological samples.

Embodiment 249. The computer program product of any one of embodiments 243-248, wherein the therapeutic agent comprises an immune checkpoint modulator.

Embodiment 250. The computer program product of any one of embodiments 243-248, wherein the therapeutic agent comprises a kinase inhibitor.

Embodiment 251. The computer program product of any one of embodiments 243-248, wherein the therapeutic agent comprises an anti-cancer chemotherapeutic.

Embodiment 252. The computer program product of any one of embodiments 243-248, wherein the therapeutic agent comprises a cell therapy.

Embodiment 253. The computer program product of any one of embodiments 243-248, wherein the therapeutic agent comprises a cancer vaccine.

Embodiment 254. The computer program product of any one of embodiments 243-248, wherein the therapeutic agent comprises an mRNA vaccine.

Embodiment 255. The computer program product of any one of embodiments 243-248, wherein the therapeutic agent comprises an RNA silencing (RNAi) agent.

Embodiment 256. The computer program product of any one of embodiments 243-248, wherein the therapeutic agent comprises a gene editing agent.

Embodiment 257. The computer program product of any one of embodiments 243-248, wherein the therapeutic agent comprises CRISPR/Cas system.

Embodiment 258. The computer program product of any one of embodiments 243-248, wherein the therapeutic agent comprises an antibody.

Embodiment 259. The computer program product of any one of embodiments 243-248, wherein the therapeutic agent comprises an RNA replacement therapy.

Embodiment 260. The computer program product of any one of embodiments 243-248, wherein the therapeutic agent comprises a protein replacement therapy.

Embodiment 261. The computer program product of any one of embodiments 231-260, wherein the database comprises normalized control gene expression values of each control biological sample of the plurality, wherein the normalized control gene expression values are calculated by a technique that comprises quantile normalization.

Embodiment 262. The computer program product of any one of embodiments 231-261, wherein the database comprises gene expression counts obtained from at least 10 control biological samples.

Embodiment 263. The computer program product of any one of embodiments 232-262, wherein the identifying, by the identifying component, comprises comparing the gene expression values of the test biological sample to gene expression values of the plurality of control biological samples.

Embodiment 264. The computer program product of any one of embodiments 232-263, wherein the gene expression values of the test biological sample and the gene expression values of the plurality of control biological samples are normalized using a common normalization technique.

Embodiment 265. The computer program product of any one of embodiments 232-264, wherein the identifying, by the identifying component, the gene that is aberrantly expressed in the test biological sample relative to the plurality of control biological samples comprises a non-parametric comparison of (i) a normalized gene expression value for a candidate gene from the test biological sample with (ii) a distribution of normalized gene expression values for the candidate gene obtained from the plurality of control biological samples.

Embodiment 266. The computer program product of any one of embodiments 232-265, wherein the method further comprises categorizing, by the gene identifying component, the gene expression values of the test biological sample, wherein categories comprise VERY LOW, LOW, NORMAL, HIGH, and VERY HIGH categories, wherein: vi. the VERY HIGH category includes genes with a gene expression value for the test biological sample that is greater than a threshold calculated based on distribution of a candidate gene's expression in the plurality of control biological samples and is lesser of: (i) a maximum gene expression value for the candidate gene in the plurality of control biological samples; and (ii) a sum of Q3 and 1.5 times IQR of gene expression values for the candidate gene in the plurality of control biological samples; vii. the HIGH category includes genes not classified in the VERY HIGH category with a gene expression value for the test biological sample that is greater than a sum of median plus two times IQR of the gene expression values for the candidate gene in the plurality of control biological samples; viii. the VERY LOW category includes genes with a gene expression value for the test biological sample that is less than a threshold calculated based on distribution of the candidate gene's expression in the plurality of control biological samples and is lesser of: (i) minimum gene expression value for the candidate gene in the plurality of control biological samples; and (ii) a difference of Q1 and 1.5 times IQR of the gene expression values for the candidate gene in the plurality of control biological samples; ix. the LOW category includes genes not classified in the VERY LOW category with a gene expression value for the test biological sample that is: (i) less than a difference of median and two times IQR of the gene expression values for the candidate gene in the plurality of control biological samples; and x. the NORMAL category is assigned to genes that are not categorized in the VERY LOW, LOW, HIGH, or VERY HIGH categories.

Embodiment 267. The computer program product of any one of embodiments 232-265, wherein the method further comprises categorizing, by the gene identifying component, the gene expression values of the test biological sample, wherein categories comprise VERY LOW, LOW, NORMAL, HIGH, and VERY HIGH categories, wherein thresholds for the categories are calculated according to a non-parametric comparison of (a) a gene expression value for a candidate gene in the test biological sample with (b) a distribution of gene expression values for the candidate gene obtained from the plurality of control biological samples using equation 1, wherein: (i) yij represents expression of gene j in sample I; (ii) mediannj is a median expression level for gene j in the plurality of control biological samples; (iii) ynjmax is maximum expression of gene j in the plurality of control biological samples; (iv) ynjmin is minimum expression of gene j in the plurality of control biological samples; (v) Q1nj is a first quartile of gene j expression in the plurality of control biological samples; (vi) Q3nj is a third quartile of gene j expression in the plurality of control biological samples; (vii) IQRnj is an interquartile range of gene j expression in the plurality of control biological samples; and (viii) mj is a range of expression of gene j in the plurality of control biological samples and is calculated using equation 2; wherein equation 1 is:

$$f ( {y}_{ij} ) = \{ \begin{matrix}VERY HIGH , & if {y}_{ij} > {median}_{nj} + 2 * {r}_{nj} \\ HIGH , & if {y}_{ij} > min ( {y}_{{nj}_{max}} , {Q}_{{3}_{nj}} + 1.5 * {IQR}_{nj} ) \\ LOW , & if {y}_{ij} < max ( {y}_{{nj}_{min}} , {Q}_{{1}_{nj}} - 1.5 * {IQR}_{nj} ) \\ VERY LOW , & if {y}_{ij} < {median}_{nj} - 2 * {r}_{nj}\end{matrix}$$

wherein equation 2 is:

rₙⱼ=min(yₙⱼ_{ₘₐₓ},Q₃_{ₙⱼ}+1.5*IQRₙⱼ)−max(yₙⱼ_{ₘᵢₙ},Q₁_{ₙⱼ}−1.5*IQRₙⱼ).

Embodiment 268. The computer program product of any one of embodiments 231-267, wherein the normalizing, by the normalizing component, further comprises applying a scaling factor to the gene expression values.

Embodiment 269. The computer program product of embodiment 268, wherein the scaling factor is calculated using a third quartile (Q3) value of the normalized gene expression values of the test biological sample.

Embodiment 270. The computer program product of embodiment 269, wherein the normalized gene expression values are divided by the scaling factor, multiplied by a scalar, and log transformed.

Embodiment 271. The computer program product of embodiment 269, wherein the normalized gene expression values are divided by the scaling factor, multiplied by 1,000, and log 2 transformed

Embodiment 272. The computer program product of any one of embodiments 231-271, wherein the test subject has a disease.

Embodiment 273. The computer program product of any one of embodiments 231-271, wherein the test subject is suspected of having a disease.

Embodiment 274. The computer program product of any one of embodiments 272-273, wherein the disease is a cancer.

Embodiment 275. The computer program product of any one of embodiments 272-273, wherein the disease is breast cancer.

Embodiment 276. The computer program product of any one of embodiments 232-275, wherein identifying, by the gene identifying component, the gene that is aberrantly expressed in the test biological sample relative to the plurality of control biological samples does not include comparing gene expression counts or normalized gene expression values from (i) a first cohort comprising the test subject and at least two additional subjects to (ii) a second cohort comprising at least three control subjects.

Embodiment 277. The computer program product of any one of embodiments 231-276, wherein the gene processing system further comprises a deduplicating component, wherein the method further comprises deduplicating, by the deduplicating component, duplicate reads identified as originating from a same RNA molecule.

Embodiment 278. The computer program product of embodiment 277, wherein the duplicate reads identified as originating from a same RNA molecule are identified based on a unique molecular identifier (UMI) appended to each RNA molecule.

Embodiment 279. The computer program product of any one of embodiments 231-278, wherein the normalized gene expression values comprise data for mRNAs.

Embodiment 280. The computer program product of any one of embodiments 231-279, wherein the normalized gene expression values comprise data for non-coding RNAs.

Embodiment 281. The computer program product of any one of embodiments 231-280, wherein the normalized gene expression values comprise data for miRNAs.

Embodiment 282. The computer program product of any one of embodiments 232-281, wherein the gene that is aberrantly expressed in the test biological sample relative to the plurality of control biological samples comprises a tumor associated antigen.

Embodiment 283. The computer program product of any one of embodiments 232-282, wherein the gene that is aberrantly expressed in the test biological sample relative to the plurality of control biological samples comprises a neoepitope.

Embodiment 284. The method of any one of embodiments 1-178, further comprising using an algorithm to identify an association between one or more of the normalized gene expression values and a clinical outcome associated with a administering a therapeutic agent.

Clause 1. A method of quantifying an RNA transcription level of one or more genes in a subject comprising extracting RNA from a biological sample from the subject, and measuring the RNA using an RNA sequencing kit comprising sequencing the RNA from the 3′-end, and identifying the RNA, thereby quantifying the RNA transcription level of the one or more genes.

Clause 2. A method of diagnosing a cancer comprising: quantifying a RNA transcription level of one or more genes in a subject comprising: extracting RNA from a biological sample from the subject, measuring the RNA using an RNA sequencing kit comprising sequencing the RNA at the 3′-end, and identifying the RNA, comparing the RNA transcription level of the one or more genes in the subject to a control RNA transcription level, and diagnosing the cancer if the RNA transcription level is different from the control RNA transcription level.

Clause 3. A method of aiding in a treatment of a cancer in a subject comprising: quantifying a RNA transcription level of one or more genes in the subject comprising: extracting RNA from a biological sample from the subject, measuring the RNA using an RNA sequencing kit comprising sequencing the RNA from the 3′-end, and identifying the RNA, comparing the RNA transcription level of the one or more genes in the subject to a control RNA transcription level, and aiding in the treatment of the cancer in the subject if the RNA transcription level is different from the control RNA transcription level, the treatment comprising administering a drug capable of modifying the RNA transcription level of the one or more genes to the control RNA transcription level.

Clause 4. The method of any one of the preceding clauses, wherein the biological sample is a saliva sample, a urine sample, a blood sample, or a tissue sample.

Clause 5. The method of any one of the preceding clauses, wherein the biological sample is formalin-fixed paraffin embedded tissue sample.

Clause 6. The method of any one of the preceding clauses, wherein the sequencing the RNA comprises a reverse transcriptase enzyme.

Clause 7. The method of any one of the preceding clauses, wherein the reverse transcriptase enzyme does not have a GC bias.

Clause 8. The method of any one of the preceding clauses, wherein the identifying the RNA comprises a unique molecular identifier (UMI).

Clause 9. The method of any one of the preceding clauses, wherein the UMI comprises Unique Molecular Identifier (UMI) Second Strand Synthesis Module for QuantiSeq FW.

Clause 10. A method of aiding in a treatment of a cancer in a subject comprising:
quantifying an RNA transcription level of one or more genes in the subject,
comparing the RNA transcription level of the one or more genes in the subject to a control RNA transcription level, and
aiding in the treatment of the cancer in the subject if the RNA transcription level is different from the control RNA transcription level, the treatment comprising administering a drug capable of modifying the RNA transcription level of the one or more genes to the control RNA transcription level.

Clause 11. The method of any one of the preceding clauses, wherein the cancer is a solid tumor.

Clause 12. The method of any one of the preceding clauses, wherein the cancer comprises lung cancer, brain cancer, breast cancer, ovarian cancer, bladder cancer, or colon cancer.

Clause 13. The method of any one of the preceding clauses, wherein the cancer is breast cancer.

Clause 14. The method of any one of the preceding clauses, wherein the breast cancer is triple-negative breast cancer.

Clause 15. The method of any one of the preceding clauses, wherein the cancer is ovarian cancer.

Clause 16. The method of any one of the preceding clauses, wherein the one or more genes comprises PARP1, PARP2, BRCA1, BRCA2, PD1, PDL1, CTLA4, CD86, DNMT1, YES1, ALK, FGFR3, VEGFA, BTK, HER2, CDK4, CDK6, ESR1, ESR2, PGR, AR, MK167, TOP2A, TIM3, GITR, GITRL, ICOS, ICOSL, IDO1, LAG-3, NY-ESO-1, TERT, MAGEA3, TROP2, CEACAM5, RB1, P16, MRE11, RAD50, RAD51C, ATM, ATR, EMSY, NBS1, PALB2, or PTEN.

Clause 17. The method of any one of the preceding clauses, wherein the one or more genes comprise at least 5, 10, 20, 30, 50, 100, 500, 1,000, or 5,000 genes.

Clause 18. The method of any one of the preceding clauses, wherein AI continuously updates the algorithm.

Clause 19. The method of any one of the preceding clauses, further comprising identifying a cancer vaccine that can benefit the subject.

Clause 20. The method of any one of the preceding clauses, further comprising designing a de novo cancer vaccine that can benefit the subject.

### EXAMPLES

### Example 1: RNA Extraction, Library Preparation, and Sequencing

### Samples

Samples of fresh frozen (FF) or formalin-fixed paraffin-embedded (FFPE) cancer tissue (e.g., breast cancer tissue, such as triple negative breast cancer tissue) and normal controls were obtained from various clinical centers. Sex, age, and sample histology information were obtained from pathology reports. For breast cancer samples, ER, PR and HER2 status was also obtained (e.g., via IHC). Select samples were subjected to IHC testing for markers AR (with AR441 clone) and CD274/PDL1 (with 28-8 clone). Fresh frozen tissue from donors with no pathologically diagnosed diseases (e.g., breast tissue from female subjects) was obtained from biobanks.

### RNA Isolation

FFPE samples: FFPE blocks and curls were stored at 4° C. in a desiccator with dry silica gel. Prior to total RNA extraction several 20 μm curls were cut from each FFPE block and placed in sterile 1.5 mL centrifuge tubes. Total RNA extraction of FFPE tumor samples was performed on two 20 μm curls using the Formapure XC Total FFPE kit (Beckman Coulter) using the manufacturer's protocol with modifications, including addition of an extra de-crosslinking step to reduce the crosslinking introduced by the formalin during the fixation process. The manufacturer's protocol included two 5-minute incubations at 80° C. prior to Proteinase K treatment for 120 minutes at 60° C. The addition of a 15-minute incubation at 80° C. for de-crosslinking after the 120-minute Proteinase K treatment led to significant improvements in the quality of sequencing data obtained from FFPE samples.

Fresh frozen samples: fresh frozen (FF) tissue samples were stored at −80° C. until total RNA extraction. Prior to total RNA extraction the samples were cut into pieces of 50-100 mg. Tissue was cryo-pulverized using the CP01 cryoPREP Manual Dry Pulverizer (PN 500230, Covaris). To capture the fresh frozen tissue fragments the sample was placed into tissueTUBE TT1 Extra Thick (XT) (SKU 520007, Covaris). The pulverized sample was mixed with 0.99 ml of RTL buffer (Qiagen) pre-mixed with 10 μL β-Mercaptoethanol (BME) and transferred to a 1 ml milliTUBE from Covaris. The pulverized sample in RTL/BME was homogenized on a Covaris M220 focused ultrasonicator using a Covaris protocol. The homogenized sample in RTL/BME was mixed with 1 ml of Trizol using the Covaris M220 focused ultrasonicator using the extraction protocol setting provided by Covaris. Trizol extraction completed the total RNA extraction from FF samples.

### DNase Treatment

RNA quantity was measured using the Qubit™ RNA HS Assay Kit on the Qubit 3 fluorometer. All RNA samples were subject to an extra DNase Treatment using Baseline Zero DNase for 30 minutes at 37° C. 2.5 μL Baseline-ZERO DNase (Luci-gen/Epicentre) was used for every 2 μg of total RNA in 50 μL reaction. Stop Solution was not added after incubation for 30 minutes and no heat-inactivation of the DNase was performed. Following the DNase treatment, the RNA was purified and concentrated using Zymo RNA Clean & Concentrator-5 RNA spin columns to provide sufficiently high RNA concentration for library generation. Total RNA was eluted in 10-12 μL DNase/RNase-free water.

### Library Preparation

The quality and quantity of RNA was evaluated prior to library preparation. Qubit chemistry was used for RNA quantification. For evaluation of RNA quality, fragment analysis was conducted using either High Sensitivity RNA ScreenTape Analysis on a Tapestation (Agilent) or the HS RNA Kit on the 5200 Fragment Analyzer System (Agilent). Fragment analyzer or bioanalyzer traces were used to calculate DV200 (DV200=[fragments >200 bases/(fragments >200 bases+fragments <200 bases)]) or DX200 (DX200=[fragments >200 bases/(fragments >200 bases+fragments <200 bases*10)]). In some embodiments, good downstream data are obtained by methods of the disclosure even if RNA with DV200 less than 30%, or DX200 less than 5%, is used as input. In some embodiments, good downstream data are obtained if DV200 is at least 30%, or DX200 is at least 4% or at least 5%.

Libraries were prepared using a method that converted mRNA to cDNA and modified the libraries to comprise a unique universal molecular identifier sequence (UMI) at the beginning of read 1 of every individual cDNA molecule, and universal dual indexes (UDI) for de-multiplexing of a pool of libraries compatible with the Illumina NGS platforms. The workflow can be adapted to other platforms/technologies including future iterations of Illumina platforms.

The amount of input material and number of PCR cycles was adjusted depending on sample quality and source. For FFPE samples, RNA input was approximately 1 μg, and the samples were subjected to 3 additional PCR cycles and an extended reverse transcription (RT) reaction. For Fresh frozen samples, RNA input was approximately 500 ng and the manufacturer's protocol was followed. All quantifications were done by Qubit chemistry.

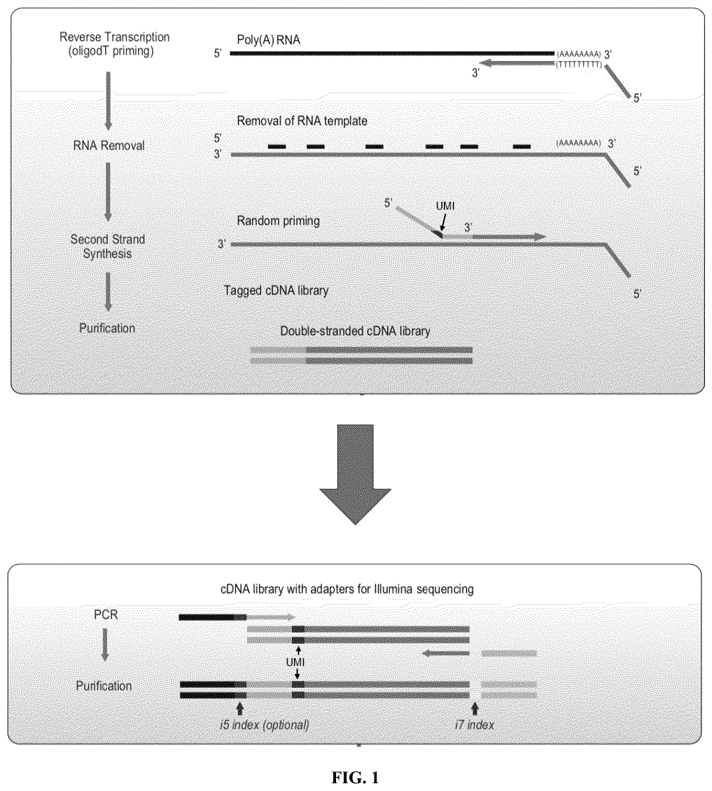

FIG. 1 illustrates generation of a cDNA library from RNA. First strand synthesis utilized oligo d(T) priming to specifically bind to poly(A) tails of mRNA transcripts. RNA template was degraded following first strand synthesis, allowing random primers to be used for second strand synthesis. During the second strand synthesis, a Unique Molecule Identifier (UMI) was incorporated to help identify PCR bias and duplicate PCR clones and reduce the impact of these on downstream analysis. The cDNA library was amplified by PCR with sequencing adapters introduced that contain unique dual indexes (UDI) that can be utilized in sequencing QC (for example, demultiplexing or filtering index-hopped reads). Samples comprising intact RNA were prepared and sequenced in separate batches from samples comprising FFPE-derived/degraded RNA.

### Sequencing

Libraries were quantified, pooled, and sequenced on the Illumina Platform (75 cycles), utilizing the sequencing-by-synthesis approach with fluorescently labeled reversible-terminator nucleotides. The platform allows samples to be multiplexed, for example, 16 samples can be multiplexed on the NextSeq 550 System to obtain a sufficient read depth for gene expression analysis. Using a MiSeq Nano sequencing kit the sequencing libraries were pooled and QC performed using equal volumes to assess the cluster efficiency of the individual sample relative to other samples in the same pool. Then this cluster efficiency measurement was used to pool the samples for a NextSeq (75 base read length) run aiming for 20 million raw reads per sample. Samples that did not reach that threshold were re-sequenced and the reads were pooled post-sequencing prior to final analysis.

As illustrated in FIG. 2, sequencing primers were utilized to generate reads in a direction equivalent to 5′ to 3′ of the original mRNA transcript such that if the sequencing read is long enough, the read would comprise the poly(A) tail in the end of read 1. Reads were also generated containing the index (e.g., universal dual index) sequences. Reads in a direction equivalent to 3′ to 5′ of the original mRNA (“read 2”) and beginning with poly(dT) (complementary to the original poly(A) tail) were not sequenced.

Replicates from each sample were sequenced on multiple sequencing runs to obtain >1 million assigned reads. Assigned reads were defined as reads obtained after alignment and removal of PCR duplicates and low-quality reads. Results from replicates that did not achieve at least 1 million assigned reads were discarded.

### Example 2: Determining Gene Expression Counts Based on Expression Data

RNA sequencing data (e.g., produced as in EXAMPLE 1) were processed using a bioinformatics pipeline. A bioinformatics pipeline is a set of software processing steps used to transform or analyze raw data. The RNA-sequencing bioinformatics analysis pipeline comprised the following steps: quality control, alignment, and transcript quantification.

### Initial Processing

The bioinformatics pipeline utilized a shell script for initial processing. The shell script utilized multiple software tools and interfaces, including BCL2FASTQ (Illumina), BaseSpace Command Line Interface (Illumina), SevenBridges Python API, and AWS command line interface.

Raw sequencing files and the sample sheet (which contained, e.g., a list of samples from a sequencing run, their index sequences, and the sequencing workflow) and run ID associated with the sequencing run were acquired and from BaseSpace Sequence Hub and input into the shell script. Sequencing (e.g., as in EXAMPLE 1) produced raw data files in binary base call (BCL) format, that were converted to FASTQ format. The shell script downloaded BCL files from BaseSpace, converted them to FASTQ, stored a copy of all sequencing files to a cloud storage service, and sent the files to a bioinformatic cloud-computing infrastructure host for further processing.

### FASTQ to Gene Expression Count

An alignment pipeline was used that comprised the following steps and software tools: de-duplication (UMI-tools), adapter sequence and quality trimming (BBduk), alignment (STAR), alignment sorting and indexing (SAMtools), and transcript quantification (HTSeq-count). FASTQC was used to collect quality control metrics prior to and after de-duplication (UMI-tools).

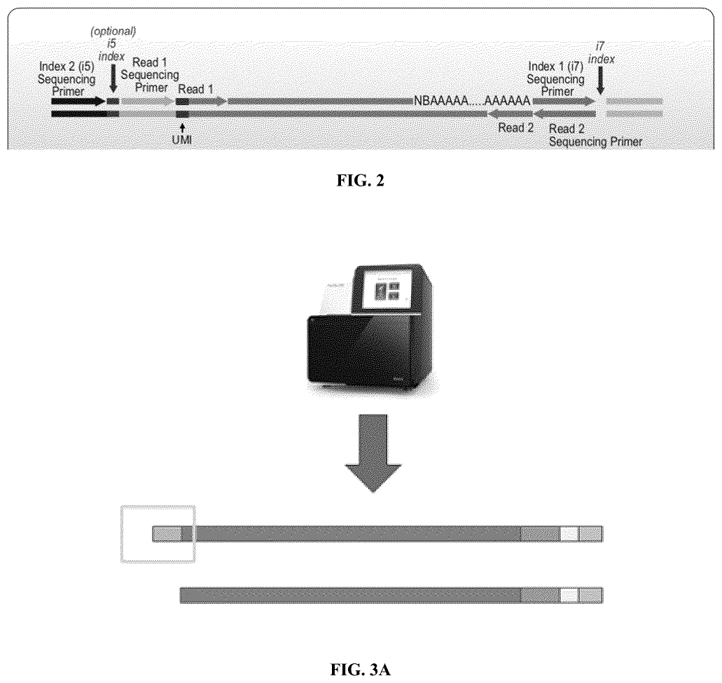

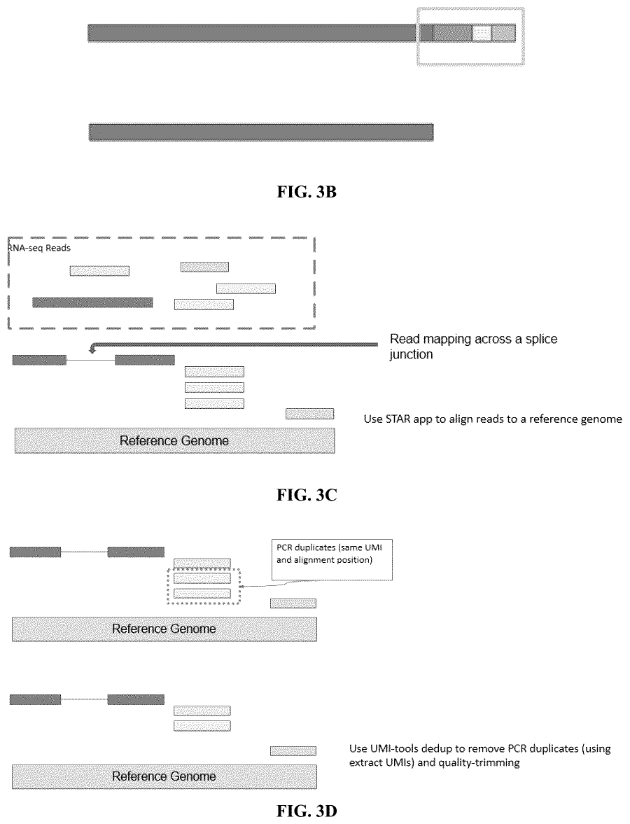

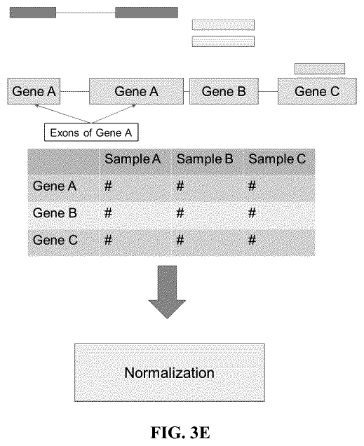

De-duplication reduces errors from PCR-introduced duplicates. UMI-tools is a tool to deduplicate sequencing reads using Unique Molecular Identifiers. UMI tools 0.5.4 was used to extract the UMIs from reads and add them to read names for a subsequent PCR de-duplication step (FIG. 3A).

Adapter sequence and quality trimming increases alignment quality by removing low quality reads and adapter sequences introduced through the library preparation steps. BBduk is an adapter trimming tool used to decrease the effect of adapter contamination on alignment of reads to a reference genome. Bbduk 38.22 was used for data-quality related trimming, filtering and masking, e.g., to trim adapters on the 3′ end and perform quality-trimming to facilitate better alignment to the reference genome (FIG. 3B).

Alignment allows for sequencing reads to be mapped to the human reference genome. STAR 2.6.0c was used to align reads from FASTQ files processed as described herein to the Genome Reference Consortium Human Build version 38 Human Genome (GRCh38) (FIG. 3C). Read alignment information was written to a BAM file format, which is a binary file format that contains sequence alignment information. SAMtools was used to sort and create an index for BAM files.

PCR duplicates containing the same UMI and alignment position were removed using UMI-tools (FIG. 3D).

Transcript quantification used the output of STAR to count how many reads map to individual genes. The result of these steps was gene expression counts for each sample. HTSeq 0.6.1 was used to quantify how many aligned sequencing reads were assigned to transcripts (FIG. 3E), resulting in gene expression count tables for each sample. Gene expression counts for samples that were biological and technical replicates were pooled to obtain a target of at least 1 million assigned reads.

### Example 3: Normalization and Identification of Aberrantly Expressed Genes

Gene expression counts (e.g., determined as in EXAMPLE 2) were further processed to identify aberrantly expressed genes (e.g., over-expressed or under-expressed genes). Aberrant expression was determined by comparing to gene expression counts obtained from RNA sequencing of corresponding normal tissue samples (control biological samples) from normal control subjects (e.g., from healthy subjects without cancer or without any known disease diagnosis). In some embodiments, the normal control subjects are matched to the test subject(s), for example, normal healthy subjects matched to test subjects with cancer based on age and/or sex.

This approach facilitates comparison of a test biological sample (e.g., a single sample) from a test subject (e.g., a single test subject) to a “reference range” established from a control group. In some embodiments, the approach also facilitates use of control data from different data sources and platforms. This method can be advantageous over many alternative methods that require paired data to be obtained from the same subject using the same platform, e.g., a cancer sample and a matched normal sample (such as PBMCs), and/or that only allow comparison between cohorts with multiple members (e.g., at least two or at least three members per cohort).

Gene expression counts were compiled in a data frame containing both tumor gene expression counts (test biological sample(s) from test subject(s) with cancer) and normal tissue gene expression counts (control biological samples from the same tissue in healthy control subjects). The data frame was normalized using the following steps and methods: (i) subsampling, (ii) normalization, and (iii) scaling using a calculated scaling factor and log 2 transformation. The normalized and scaled gene expression values from the control samples were then used to establish thresholds to identify aberrant expression for each gene of interest.
(i) Subsampling comprised use of an R package (subSeq) to subsample to a target number of assigned reads (read depth) per sample, for example 1-6 million assigned reads per sample, by utilizing binomial sampling. A target of 6 million assigned reads was used for breast tissue.
(ii) Gene expression counts were normalized in the following manner: 1) data for each sample was sorted to rank the non-zero gene expression counts assigned to each gene of the test biological sample from lowest count to highest count. This was done for all samples. 2) For each position of the sorted gene expression counts of the sample, an average gene expression value was calculated for all samples as the avg_position_x=sum_counts_x/count_samples (i.e., a mean was calculated for the lowest gene expression count in all samples, a mean was then calculated for the 2nd lowest gene expression count in all samples, etc.). The output was a list of ordered averages calculated from all samples. The list was then used to update gene expression counts in each sample with the ordered average value with the same rank (i.e., the lowest gene expression count in a sample was replaced by the lowest ordered average, the second lowest gene expression count was replaced by the second lowest ordered average, etc.).

TABLE 1 provides an example and illustrates that total gene expression count for each sample is the same after normalization. The unique values for gene expression counts within each sample are the same after normalization.

TABLE 1

| | Sample1_{—} | Sample1_{—} | Sample2_{—} | Sample2_{—} | | |
|---|---|---|---|---|---|---|
| | count | pos_asc | count | pos_asc | Sample1_norm | Sample2_norm |
| Gene1 | 0 | 1 | 50 | 2 | 10 [=(0 + 20)/2] | 27.5 [=(5 + 50)/2] |
| Gene2 | 5 | 2 | 20 | 1 | 27.5 [=(5 + 50)/2] | 10 [=(0 + 20)/2] |
| Gene3 | 10 | 3 | 70 | 4 | 25 [=(10 + 40)/2] | 45 [=(20 + 70)/2] |
| Gene4 | 20 | 4 | 40 | 3 | 45 [=(20 + 70)/2] | 25 [=(10 + 40)/2] |

(iii) Scaling and transformation of gene expression comprised use of an R-script to scale normalized gene expression values by a scaling factor. The scaling factor was calculated by ranking gene expression for each sample. The 75^{th} percentile/third quartile (Q3) for each sample was then used to calculate a mean (Q3 mean) of all the samples. The scaling factor was then calculated using the following equation:

f_s=(Q3_mean*1,000)+1.

All normalized gene expression values were divided by the scaling factor f_s, and resulting values were then log 2 transformed. After log 2 transformation, the majority of normalized gene expression values fall within a 0 to 20 point scale.

Aberrant gene expression was detected using thresholds set by gene expression in healthy tissue for all genes. For each gene, expression in the test biological sample was compared to the distribution of expression in normal tissue (control biological samples). The distribution of expression in normal tissue for each gene was described by the median, first quartile (Q1), third quartile (Q3), and interquartile range (IQR) of the normalized gene expression values of the given gene. The IQR was calculated as the difference of the first quartile (Q1) and third quartile (Q3) expression values of the given gene.

Once the descriptive values of distribution were determined for the normal tissue samples, thresholds were calculated for VERY LOW, LOW, NORMAL, HIGH, and VERY HIGH expression calls. For each tumor sample and each gene of interest, the normalized expression levels were compared to the threshold values and then categorized as VERY LOW, LOW, NORMAL, HIGH, or VERY HIGH according to Equation 1 and Equation 2.

The VERY HIGH label was given to a gene expression value greater than (i) the maximum expression value of the gene in normal tissue (control samples); or (ii) the sum of the Q3 of the gene and 1.5×IQR of the gene in normal tissue (control samples). The threshold used was whichever of (i) and (ii) was the minimum value.

The HIGH label was given to a gene expression value that was (i) greater than the sum of the median and twice the IQR of the gene in normal tissue (control samples); and (ii) not categorized as VERY HIGH.

The VERY LOW label was given to a gene expression value less than (i) the minimum expression value of the gene in normal tissue (control samples); or (ii) the difference of the Q1 of the gene and 1.5×IQR of the gene in normal tissue (control samples). The threshold used was whichever of (i) and (ii) was the minimum value.

The LOW label was given to a gene expression value that was (i) less than the difference of the median and twice the IQR of the gene in normal tissue (control samples); and (ii) not categorized as VERY LOW.

A gene in a given sample was labelled as NORMAL if the expression fell between the LOW and HIGH thresholds (i.e., it was not categorized as VERY HIGH, HIGH, LOW, or VERY LOW).

Categorization of a gene as VERY HIGH, VERY LOW, HIGH, or LOW can further be described by the following equations:

$$\begin{matrix}f ( {y}_{ij} ) = \{ \begin{matrix}VERY HIGH , & if {y}_{ij} > {median}_{nj} + 2 * {r}_{nj} \\ HIGH , & if {y}_{ij} > min ( {y}_{{nj}_{max}} , {Q}_{{3}_{nj}} + 1.5 * {IQR}_{nj} ) \\ LOW , & if {y}_{ij} < max ( {y}_{{nj}_{min}} , {Q}_{{1}_{nj}} - 1.5 * {IQR}_{nj} ) \\ VERY LOW , & if {y}_{ij} < {median}_{nj} - 2 * {r}_{nj}\end{matrix} & Equation 1 \\ {r}_{nj} = min ( {y}_{{nj}_{max}} , {Q}_{{3}_{nj}} + 1.5 * {IQR}_{nj} ) - max ( {y}_{{nj}_{min}} , {Q}_{{1}_{nj}} - 1.5 * {IQR}_{nj} ) & Equation 2\end{matrix}$$

wherein:
(i) yᵢⱼ represents expression of gene j in sample i;
(ii) medianₙⱼ is a median expression level for gene j in the plurality of control biological samples;
(iii) yₙⱼₘₐₓ is maximum expression of gene j in the plurality of control biological samples;
(iv) yₙⱼₘᵢₙ is minimum expression of gene j in the plurality of control biological samples;
(v) Q₁ₙⱼ is a first quartile of gene j expression in the plurality of control biological samples;
(vi) Q₃ₙⱼ is a third quartile of gene j expression in the plurality of control biological samples;
(vii) IQRₙⱼ is an interquartile range of gene j expression in the plurality of control biological samples; and
(viii) rₙⱼ is a range of expression of gene j in the plurality of control biological samples and is calculated using equation 2.

### Example 4: Sequencing and Bioinformatics of Fresh Frozen Samples by a Control Method

Fresh frozen (FF) samples processed in EXAMPLE 1 were also processed and analyzed by a separate control method for comparison and validation of methods disclosed herein. RNA extraction and library preparation were done using an Illumina TruSeq protocol used in the Genotype-Tissue Expression (GTEx project). This technique sequences total RNA, is non-stranded, uses polyA+ selection, and like many control/alternative methods to those disclosed herein, is not FFPE compatible. Sequencing was done on the Illumina MiSeq Platform. Samples were sequenced to obtain >25 million assigned reads (i.e., reads mapped to genomic features).

After sequencing, the raw data files were downloaded and used as inputs to the GTEx alignment pipeline. The GTEx pipeline includes the following steps and software tools: input of FASTQ files, alignment (STAR v2.5.3), identification of duplicates (Picard markduplicates), quality control (RNA-seQC v.1.1.9) and transcript quantification (RSEM v1.3.0). RSEM gene expression estimates were used for downstream steps. Dockerfile for the GTEx RNA-seq pipeline was obtained from https://hub.docker.com/r/broadinstitute/gtex_maseq/. GRCh38/hg38 reference genome was used to define transcripts. The control data sets were normalized and scaled using the methods disclosed in EXAMPLE 3.

To call aberrant expressed genes for TruSeq-FF samples, RNA-seq data for 168 normal breast samples from the Genotype-Tissue Expression project obtained from the NCI Genomic Data Commons Data Portal was used as the healthy control dataset to set thresholds. Samples were filtered for samples from breast tissue, female subjects, and samples included in the GTEx Analysis Freeze. The GTEx Analysis Freeze subset are true normal samples excluding samples from donors considered “biological outliers” e.g. samples that did not pass quality-control, donors with pathological disease diagnoses, etc. The resulting true normal samples were used to set expression thresholds for the analysis to compare tumor expression to normal tissue expression.

### Example 5: Correlation of Gene Expression Results Obtained from FFPE Samples and Fresh Frozen Samples

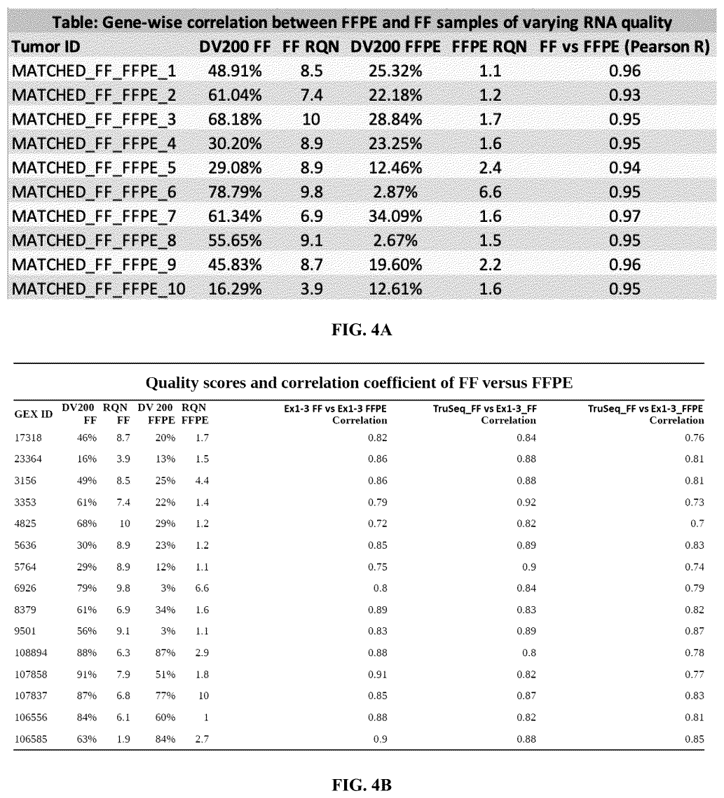

Matched FFPE and fresh frozen (FF) breast cancer samples were processed according to the methods of EXAMPLES 1-3. Gene-wise Pearson correlation (Pearson R) was calculated between data originating from the FF and FFPE breast cancer samples. As shown in FIG. 4A, FFPE and FF samples processed by these methods exhibited a high correlation (>=0.93) regardless of RNA-quality (RQN, DV200), demonstrating that these methods produce high quality results from FFPE samples as well as FF. In contrast, many alternative workflows do not produce high quality results from samples (e.g., FFPE) with a DV200<30%.

In an additional experiment, matched FFPE and fresh frozen (FF) breast cancer samples from 15 donors were processed according to the methods of EXAMPLES 1-3. Gene-wise Pearson correlation (Pearson R) was calculated between data originating from the FF and FFPE breast cancer samples. As shown in FIG. 4B, sixth column, FFPE and FF samples processed by these methods exhibited a high correlation even for samples with low RNA-quality (RQN, DV200), demonstrating that these methods produce high quality results from FFPE samples as well as FF.

### Example 6: Correlation of Gene Expression Results Obtained Using a Method of the Disclosure to Gene Expression Results Obtained Using a Control Method

The ability of a method of the disclosure to yield results comparable to a control gene expression technique was evaluated. Data generated from FF or FFPE samples according to EXAMPLES 1-3 was compared to data generated from matched pair FF samples according to the methods of EXAMPLE 4.

Pearson correlation coefficient was calculated between the two methods. Positive correlation coefficients were observed for data generated from either FF or FFPE sources using a method of the disclosure compared to the control method (FIG. 4B, rightmost two columns). The matched pairs data achieved an overall median Pearson correlation coefficient value of 0.86, representing a strong positive correlation.

Heat maps were generated showing gene expression valued determined by each method for a panel of genes identified as relevant to cancer therapeutics (e.g., genes that are markers or targets as described in EXAMPLE 11). It can be visually observed that gene expression profiles are similar in the dataset generated from FFPE samples by a method disclosed herein compared to the dataset generated from FF samples by TruSeq (FIG. 15).

These results indicate that a method disclosed herein can generate comparable gene expression data as a control method, even when the data originate from inferior quality RNA (e.g., from FFPE samples rather than FF samples).

### Example 7: Correlation of Gene Expression Results Obtained from FFPE to Immunohistochemistry Data

Immunohistochemistry (IHC) is clinically used to measure expression of key biomarkers in FFPE samples from tumor biopsies to guide treatment decisions, although the method has a number of limitations (e.g., requires specific antibodies for each target, and few data points can be obtained from any sample/section).

IHC results were collected for breast cancer samples evaluated for ER (n=10), PR (n=10), and HER2 (n=9). The samples were scored by the pathologist as positive, weakly positive, or equivocal. Select samples also had IHC done using the antibody clones AR441 and 28-8 for AR (n=4) and PDL1 (n=6), respectively. Samples were considered positive for AR or PDL1 if percent cell positivity was greater than 95%. Samples from the same donors were processed to obtain RNA sequencing data and normalized gene expression values according to the methods of EXAMPLES 1-3. Samples were considered positive for the biomarkers if the gene corresponding to the protein of interest was categorized as HIGH or VERY HIGH according to the criteria in EXAMPLE 3.

Expression data was compared to IHC data from the same samples to determine whether the RNA seq methods could predict expression of biomarkers according to IHC.

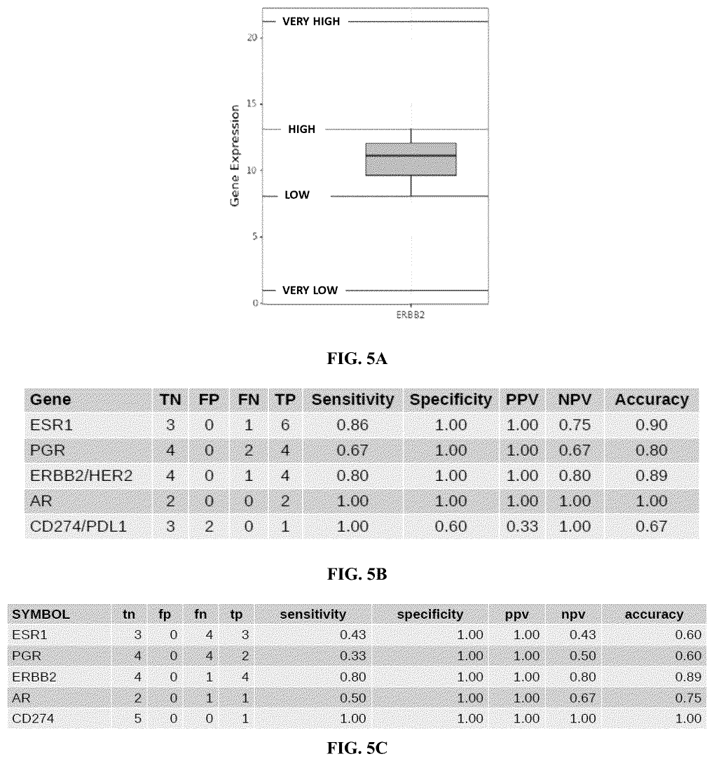

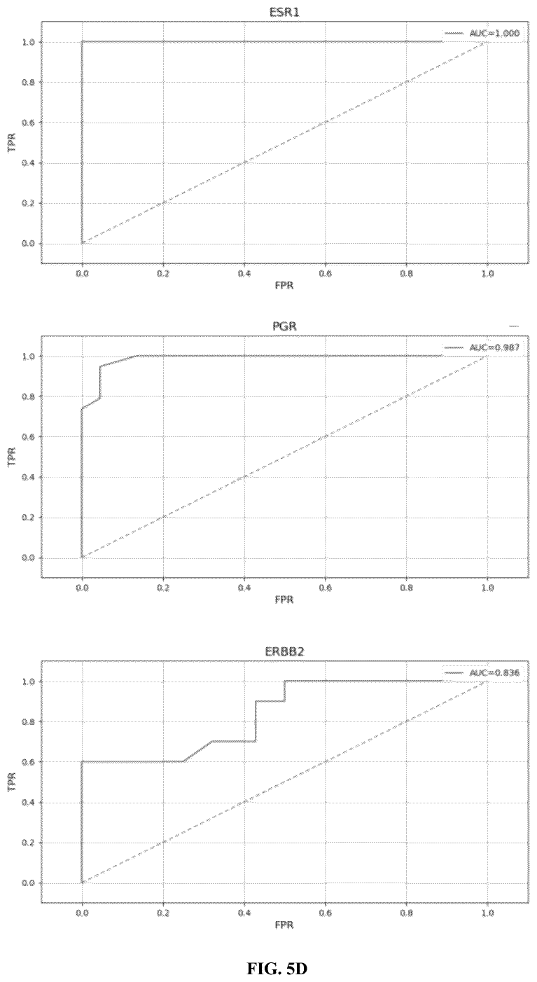

RNA expression data generated by a method of the disclosure predicted IHC status with moderate to high sensitivity and specificity (FIG. 5B). PDL1 displayed lower specificity, likely due to the small number of samples with IHC performed for this marker (n=6). In some embodiments, specificity is increased by performing PDL1 IHC on more samples.

Receiver operator characteristic (ROC) curves were generated and the area under the curve (AUC) was also calculated for ER, PR and HER2. AUC scores of 0.5 can denote a poor classifier and a score of 1 can denote a perfect classifier. AUC for ER, PR, and HER2 were about 0.85 or greater, which indicates that a method disclosed herein has a high ability to accurately predict and discriminate between negative and positive IHC results for ER, PR, and HER2 (FIG. 5D, top panel: ESR (ESR1), AUC=1; middle panel: PR (progesterone receptor/PGR), AUC=0.987; lower panel: HER2 (ERBB2), AUC=0.836). These results indicate that a method disclosed herein can reliably determine status of established clinical biomarkers. In addition, the nature of RNA sequencing allows for expression status of numerous other genes to be concurrently determined, and the expression status of such genes can have implications for diagnosis, prognosis, and treatment selection beyond the classic biomarkers.

As a control, the analysis was repeated using control biological samples that were normal adjacent tissue (NAT) from the same (test) subjects, rather than normal tissue from normal control subjects. Use of the NAT control data set to set thresholds for aberrant expression resulted in reduced accuracy and sensitivity (FIG. 5C) compared to the normal tissue from normal control subjects (FIG. 5B).

### Example 8: Cancer-Testis Antigen Expression in FFPE Breast Cancer Samples

RNA seq methods of the disclosure can detect differential expression of a diverse range of potential therapeutic targets, including, for example, neoepitopes, which are mutated antigens produced by gene mutations specific to individual tumors; tumor-specific antigens (TSA), which are uniquely expressed in tumor cells; and tumor associated antigens (TAA), which have elevated expression on tumor cells and lower expression in healthy tissues.

Cancer-Testis Antigens (CTA) are a category of TAA that have potential as therapeutic targets due to their restricted expression in normal tissue and high immunogenicity. Thus, CTA are promising targets for the development of cancer vaccines, and potentially other therapeutics.

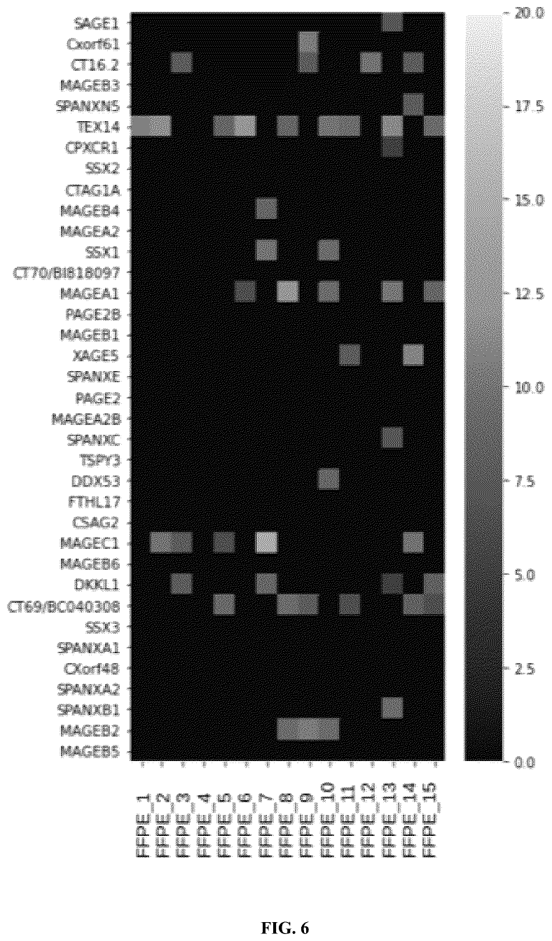

Expression of CTA genes in breast cancer samples was evaluated. CTA genes were obtained from CTDatabase, a curated database of testis-cancer antigens, and CTAs were identified by filtering the data set for testis-restricted antigens. Normalized CTA gene expression in from FFPE samples processed according to EXAMPLES 1-3 was used to determine expression of CTAs. Expression of MAGE genes was detected in 73% samples (FIG. 6). MAGE expression has been associated with tumor progression in primary breast tumors. The results of such an analysis that identifies neoepitopes, TSA, and/or TAA (e.g., CTA) in a cancer biopsy can be output into a report to suggest potential clinical courses of action (e.g., relevant therapies or therapeutic targets can be included in a treatment recommendation).

The results of such an analysis that matches identified neoepitopes, TSA, and/or TAA (e.g., CTA) in a cancer biopsy to clinical trials can be output to a report to suggest potential suitable clinical trials a subject could benefit from.

### Example 9: Therapeutic Options Based on RNA Sequencing Data

Approximately 20% of breast cancers are triple negative (TNBC), an aggressive form of breast cancer with an overall survival rate of 63%. Treatment options are limited for these patients, with no effective specific targeted treatment available for TNBC. Cancer vaccines could be used to activate and recruit the host immune system to induce anti-tumor activity by introducing cancer-specific molecules to a patient, but there remain substantial challenges for cancer vaccines to be implemented in clinical practice, for example, identification of suitable tumor antigens that are expressed in a given tumor.

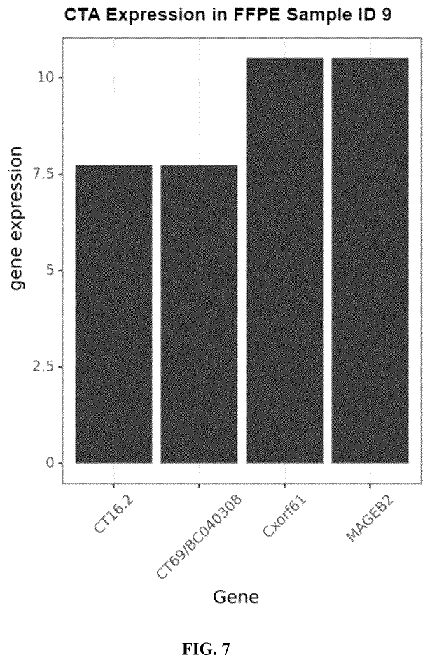

In a TNBC FFPE sample, 4 cancer testis antigens were detected using methods disclosed herein (CT16.2, CT69, CXorf69, MAGEB2; FIG. 7). CXorf61 and MAGEB2 are promising targets for cancer vaccines. CXorf61 has been identified in the basal subtype of breast cancer in TCGA RNA-seq datasets and has also been found to be expressed on the protein level, and displays immunogenic properties. A study has also demonstrated that a MAGEB1/2 DNA vaccine was effective in controlling metastasis in a mouse breast tumor model. CT16.2 and CT69 have been identified as cancer-testis associated transcripts. CT16 has been suggested to promote cell survival in melanoma cells.

These data suggest that RNA seq analysis according to methods of the disclosure (e.g., from FFPE tumor samples) can be used to identify target antigens expressed in a subject's cancer that could be administered as part of a cancer vaccine (e.g., an existing cancer vaccine, a cancer vaccine that is being tested in a clinical trial, or a de-novo generated personalized cancer vaccine, such as an mRNA vaccine). Because of the ability to rapidly develop and manufacture an mRNA vaccine (e.g., a customized/personalized vaccine), such mRNA cancer vaccines based on RNA sequencing data of tumor samples could provide effective therapies for patients with otherwise few or no remaining clinical options. Identified neoepitopes, cancer specific antigens, or tumor associated antigens could also serve as a basis for the design of novel cancer vaccines applicable to multiple patients. The results of such an analysis can be output into a report that identifies (e.g., lists or ranks), for example, potential therapeutic targets or options for a subject, including cancer vaccines that have previously been developed, or antigens that could be utilized in a de novo generated cancer vaccine.

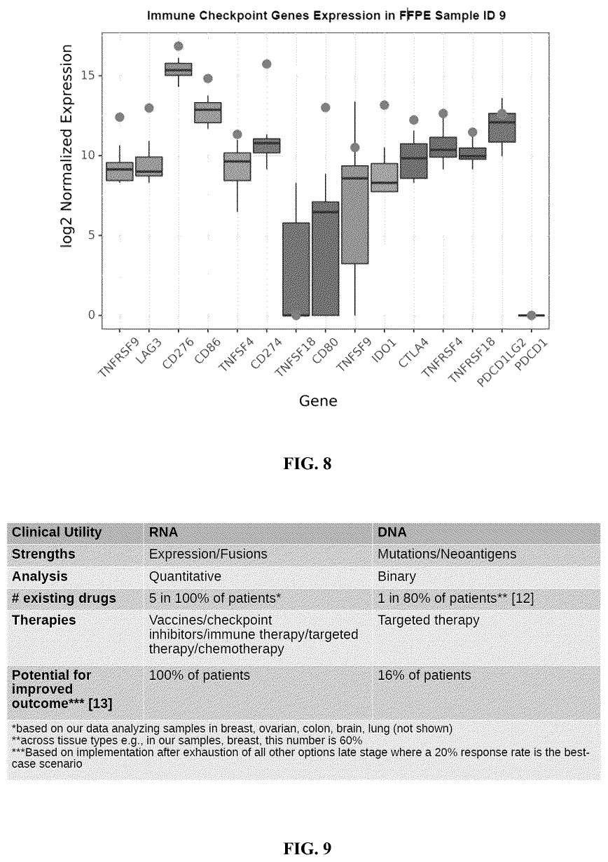

The TNBC FFPE sample also showed very high or high expression of genes involved with immune checkpoints (FIG. 8) according to a classification scheme disclosed herein (for example, as illustrated in FIG. 5A). Notably, PDL1 (CD274) was significantly over-expressed in the RNA seq data, and in IHC was found to exhibit 98% cell positivity. This indicates that anti-PD-1 therapy—such as Atezolizumab—could exert anti-tumor activity on this tumor, and that methods disclosed herein can be used to match candidate therapeutics to subjects.

The combination of immune checkpoint inhibitors and cancer vaccines has been suggested to benefit TNBC patients, and early-stage clinical trials are underway (e.g., NCT04024800 and NCT03362060). The results of an analysis such as this can be output into a report that identifies (e.g., lists or ranks), for example, potential therapeutic targets, options, or combination therapies for a subject (including, e.g., clinical trials the subject could benefit from).

These data suggest that RNA analysis according to methods of the disclosure (e.g., from FFPE tumor samples) can be used to design an effective clinical strategy incorporating two or more therapies for a given subject, e.g., by combining a cancer vaccine incorporating an antigen expressed by the cancer with a checkpoint inhibitor targeting an immune checkpoint protein expressed by the cancer, and/or other drugs.

These data further suggest that actionable insights can be generated from RNA seq data generated by methods of the disclosure from a single biopsy, e.g., without a matched normal control.

Compared to DNA sequencing based methods, the RNA sequencing based methods disclosed herein can provide insights for a broader range of potential therapeutic targets, for example, by identifying aberrantly expressed tumor associated antigens (e.g., CTA), cancer specific antigens, neoepitopes, immune targets, and immune checkpoint genes, and targets for traditional targeted therapies, many of which cannot be identified (or expression or lack thereof identified) by DNA sequencing. Furthermore, combinations of identified candidate therapeutic agents for a given subject could lead to improved likelihood of a positive outcome compared to monotherapies. Non-limiting examples of advantages of methods disclosed herein compared to DNA-based methods are provided in FIG. 9.

### Example 10: Database of Therapeutic Targets, Therapeutics, and Clinical Trials

A curated database of mRNA transcripts that are associated with particular cancer treatments, drug targets, and clinical trials is generated. The database can include individual mutations, over/under-expressed genes, tumor associated antigens (TAA, e.g., cancer testis antigens (CTA)), neoepitopes, tumor specific antigens (TSA), and/or gene expression signatures, that are associated with specific cancer therapies and clinical trials. Transcripts of interest identified by methods of the disclosure, for example, TAA (e.g., CTA), neoepitopes, or TSA, can be queried against the database that contains information about potentially suitable therapeutics and/or clinical trials. Potential therapies, combination therapies, and clinical trials that could benefit a subject can be identified, and the results can be output into a report.

### Example 11: Database of Therapeutic Targets, Therapeutics, and Clinical Trials

A curated database of cancer therapeutics and genes encoding markers and targets associated with the cancer therapeutics was generated. The database was designed to be suitable for use with methods of the disclosure to provide wellness recommendations, e.g., that comprise additional insights and treatment recommendations compared those that rely on the small number of conventional biomarkers in clinical use.

The database was created through the manual curation of cancer therapeutics from the National Cancer Institute (NCI) and DrugBank for gene markers and targets. Cancer treatments and therapeutics were imported from the NCI and pharmacological information was imported from DrugBank. Curators with backgrounds in genetics and biology determined targets and markers for each therapeutic. For the purposes of the database, targets were molecules in the body associated with a disease indication that can be targeted by a therapeutic. For the purposes of the database, markers were molecules that are part of an inclusion or exclusion criterion for a particular treatment. Curators used information from DrugBank to categorize therapeutics (e.g., immunotherapy, hormone therapy, etc.). Information submitted by the curators was subject to a review process.

Additional standard of care biomarkers were obtained from the 2019 National Comprehensive Cancer Network (NCCN) Biomarker Compendium®, that contains expression-based molecular abnormalities related to prognosis or treatment for various cancer types such as breast, ovarian, lymphoma etc.

159 genes were identified that encode targets and markers for approved cancer treatments. This was greater than the number of biomarkers available through the NCCN biomarker Compendium® (108), and little overlap was observed between the two datasets (12 genes).

### Example 12: Identification of Over-Expressed Tumor Antigens Targeted by Existing Therapies and Use of Cohort Data to Design Clinical Trials

RNA seq data for triple negative breast cancer (n=123) and normal breast tissue controls (n=67) were obtained from the Cancer Genome Atlas Breast Invasive Carcinoma (TCGA-BRCA) data collection. The RNA seq data was processed according to the methods in EXAMPLE 3.

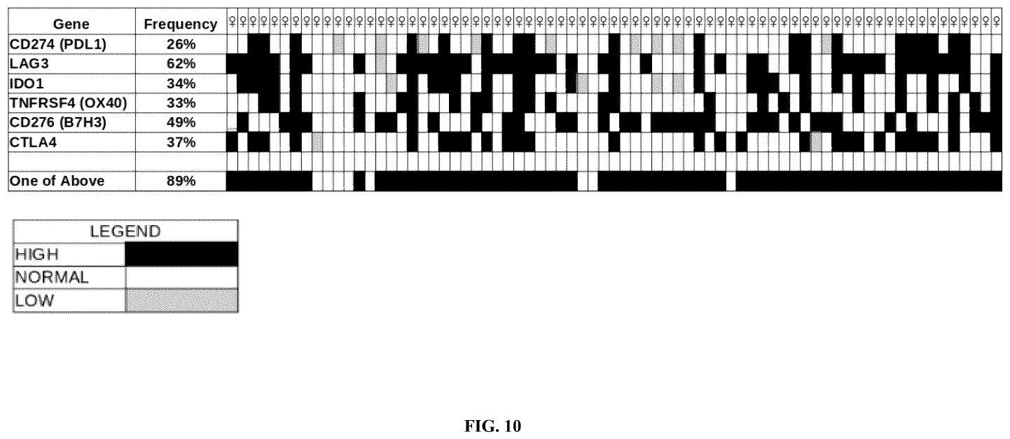

Most samples over-expressed several tumor antigens targeted by emerging immune therapies (FIG. 10), e.g., PDL1, LAG3, IDO1, OX40, B7H3, and/or CTLA4. Over-expressed immune checkpoint gene(s) were identified in >80% of TNBC samples. This suggested profiling CTA and checkpoint genes could benefit TNBC patients, for example, by identifying patients that would benefit the most from certain therapies, such as integrative treatments of cancer vaccine and checkpoint inhibitors. These data could also be used to connect patients to suitable clinical trials. The results of analyses can be output to a report.

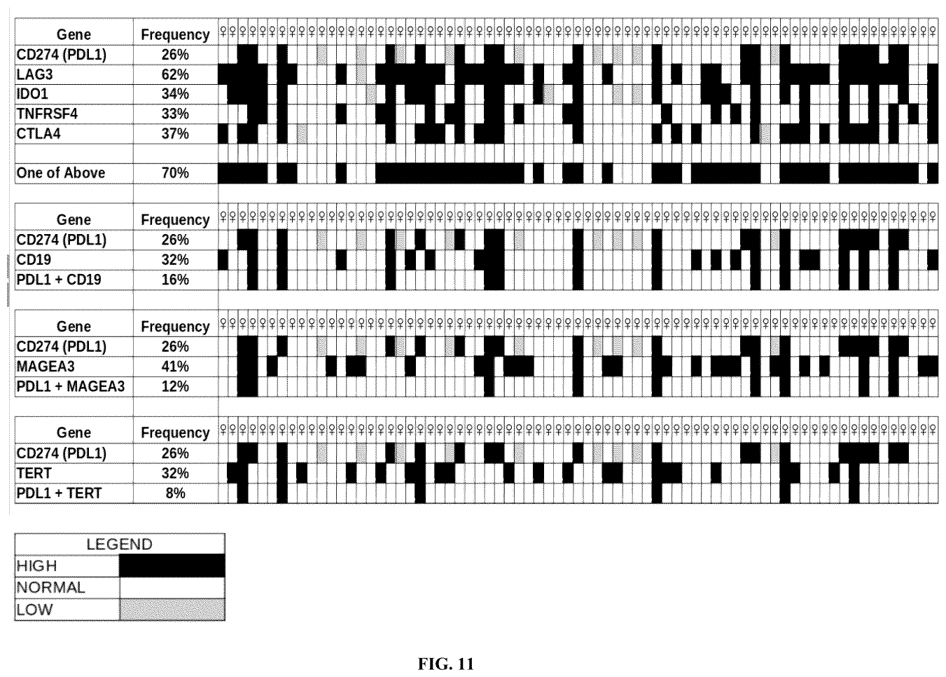

The results were also used to design a hypothetical combinatorial study with 3 immune therapy targets and 1 checkpoint inhibitor (anti-PDL1). Design was able to “enroll” 30% of the TNBC population based on the frequency of altered expression (FIG. 11). This outcome suggests that effective clinical trial design and/or enrollment can be achieved using methods of the disclosure, whereas enrollment based on mutations identified by DNA sequencing can be difficult due to a low population penetrance of a given mutation.

These results also show that methods of the disclosure can be applied to raw data generated from various sources and platforms, e.g., including use of normal control data and/or cancer sample data from existing RNA-seq datasets.

### Example 13: RNA Transcription Level of EGFR in a Breast Tumor

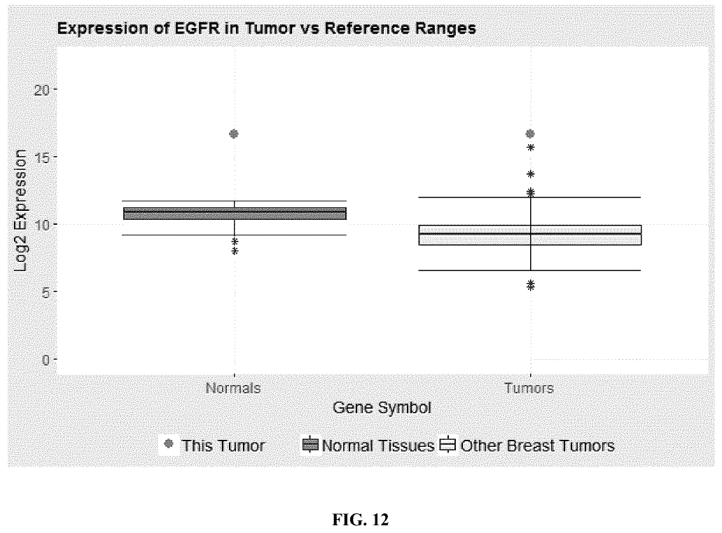

FIG. 12 shows the log 2 RNA expression of EGFR in breast cancer tissue samples and normal controls processed by methods of the disclosure. As compared to control RNA transcription in normal tissue (left), the RNA transcription level is outside of the expected range for EGFR expression in normal tissue for some of the tumor samples, including the one labeled by the symbol for “this tumor”. As compared with RNA transcription in other reference tumor tissue (right), the RNA transcription level of the sample labeled “this tumor” is comparable to a high sample in the reference data set and outside of the expected RNA expression level of EGFR in breast cancer.

### Example 14: RNA Transcription Level of a Panel of Genes in Cancerous and Normal Breast Tissue

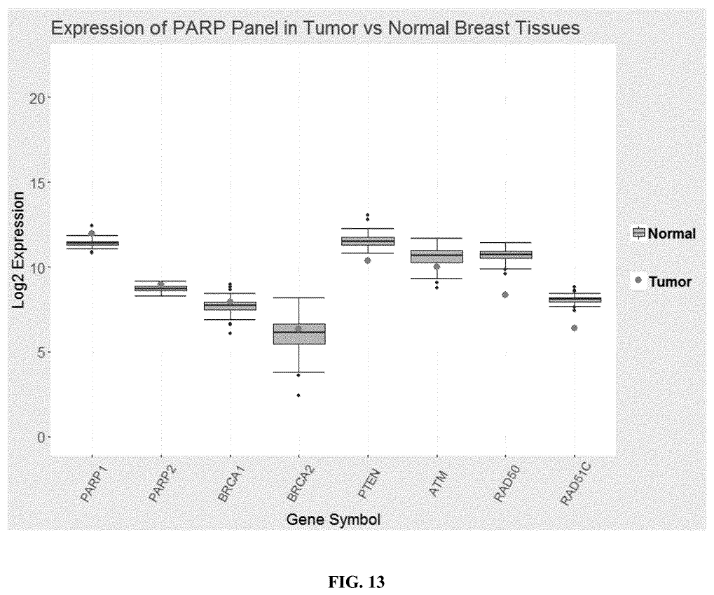

FIG. 13 shows the log 2 RNA expression level of a panel of genes, including PARP1, PARP2, BRCA1, BRCA2, PTEN, ATM, RAD50, and RAD51C, in a breast cancer tissue sample as compared to the range shown for normal breast tissue, processed by methods of the disclosure. Based on the results for this tissue sample, the RNA expression levels are high for PARP1; and low for PTEN, RAD50, and RAD51D. The results were queried in a curated database of mRNA transcripts that are associated with particular cancer treatments, drug targets, and clinical trials, and a report generated listing tumor expression state, clinical relevance, and matched clinical trials the subject could benefit from.

The results were output into a report comprising the information shown in in Table 2.

TABLE 2

| | Tumor | | |
|---|---|---|---|
| | Expression | | Matched |
| Gene(s) | State | Clinical Relevance | Trials |
| PARP1 | High | PARP1 expression levels are being evaluated as a | NCT01351909 |
| | | biomarker for response to the PARP inhibitor | |
| | | veliparib. | |
| PTEN | Low | PTEN deficiencies can be a sensitizing factor for | NCT02401347, |
| | | PARP inhibitors (Dillon 2014, Mendez-Pereira | NCT01884285 |
| | | 2009). Ongoing clinical trials for PARP inhibitors | |
| | | select patient with known PTEN deficiencies and | |
| | | mutations. | |
| RAD50, | Low | Deficiencies in RAD50, RAD51D are being | NCT02401347 |
| RAD51D | | explored as sensitizing factors for PARP inhibitors | |
| | | in triple negative breast cancer | |

### Example 15: Concordance of RNA Expression Results with Immunohistochemistry

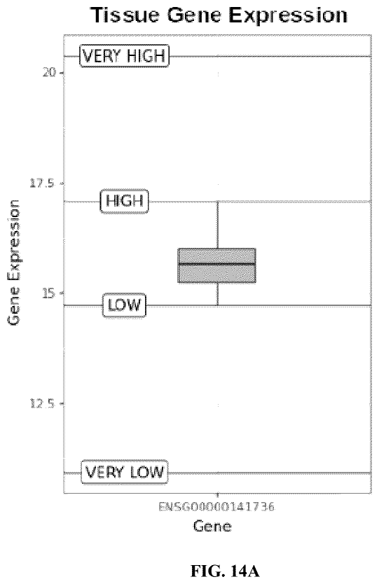

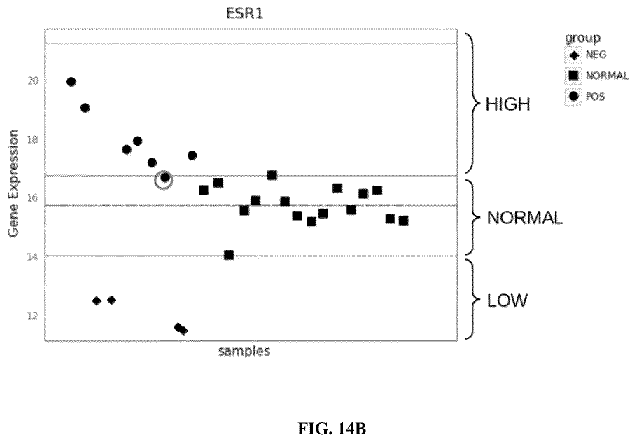

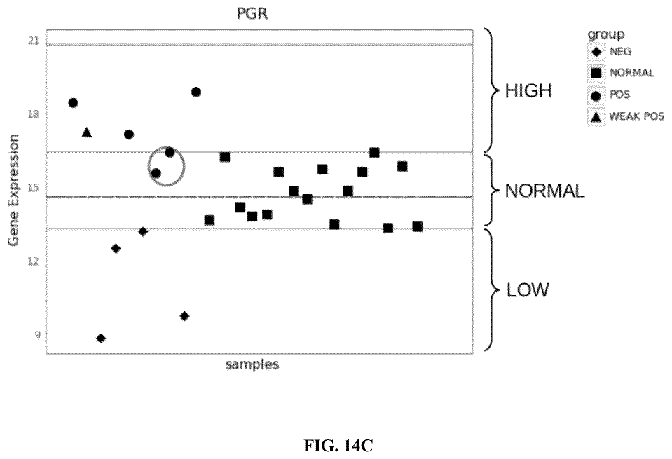

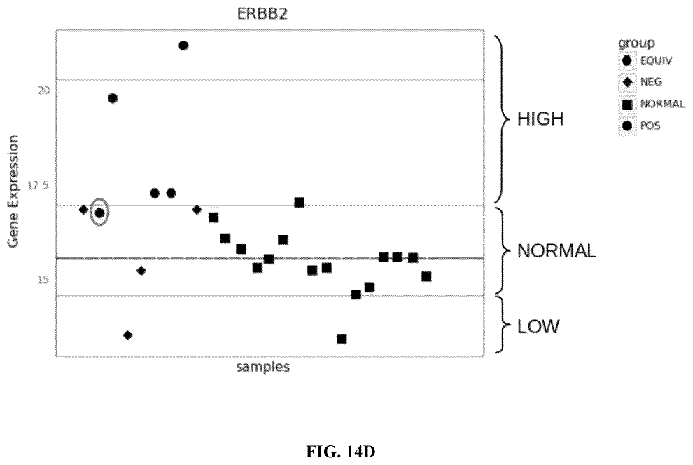

16 normal breast tissue samples were used for a healthy control dataset generated according to the methods of EXAMPLES 1-3. 15 samples of breast cancer tissue were processed according to the methods of EXAMPLES 1-3, and normalized gene expression values were categorized as VERY LOW, LOW, NORMAL, HIGH, or VERY HIGH according to Equation 1 and Equation 2, with the 16 normal healthy breast tissue samples used as the control biological samples to set the categorization thresholds. An illustrative plot showing thresholds relative to normal tissue gene expression for HER2 is provided in FIG. 14A. Samples were considered positive for the biomarkers if the gene corresponding to the protein of interest was categorized as HIGH or VERY HIGH according to the criteria in EXAMPLE 3. Paired IHC samples were scored by the pathologist as positive, weakly positive, normal, negative, or equivocal.

Data for ER (ESR1), PR (PGR), and HER2 (ERBB2) are shown in FIGS. 14B, 14C, and 14D, respectively, with the “group” legend indicating IHC status of the sample.

Nine of the samples showed perfect concordance among replicates for categorizing ER, PR, and HER2, as shown in TABLE 3.

TABLE 3: reproducibility of replicates for categorizing expression levels of ER, PR, and HER2 in replicates of breast cancer samples. The denominator is the number of replicates and the numerator is the number of replicates that are in agreement.

TABLE 3

| Sample | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| ER | 2/2 | 2/2 | 2/2 | 2/2 | 2/2 | 1/2 | 3/3 | 2/2 | 2/2 | 3/3 | 3/3 | 3/3 | 3/3 | 3/3 | 3/3 |
| PR | 2/2 | 2/2 | 2/2 | 2/2 | 2/2 | 1/2 | 3/3 | 2/2 | 1/2 | 3/3 | 3/3 | 3/3 | 3/3 | 3/3 | 3/3 |
| HER2 | 2/2 | 2/2 | 2/2 | 1/2 | 2/2 | | 3/3 | 1/2 | 1/2 | 3/3 | 3/3 | 3/3 | 3/3 | 3/3 | 3/3 |

Samples with discordant results were samples where gene expression for a particular gene fell on the border of a categorization threshold (e.g., the circled values in FIGS. 14B, 14C, and 14D).

It was noted that high quality samples (DV200>50%) show perfect concordance for ER, PR and HER2, however concordance was also achieved for samples with low DV200 samples.

### Example 16: Algorithm Combining Normalized Gene Expression Values with Clinical Data

Normalized gene expression values determined by methods disclosed herein are compiled into a database. The database also includes clinical characteristics, such as age, sex, diagnosis (e.g., cancer type, cancer lymph node involvement), biomarker status, and other parameters. The database includes data regarding clinical outcome, e.g., whether a given subject is a responder or non-responder to a treatment that was administered.

An algorithm is used to associate the gene expression values with the clinical data and responder status. The algorithm uses machine learning to associate gene expression values and combinations thereof to clinical outcome data (e.g., responder vs non-responder status for a given treatment). The algorithm can be updated as new data become available, e.g., for new therapeutics as they are tested and become approved.

Using gene expression data (e.g., quantitative normalized gene expression values, categorizations of gene expression levels disclosed herein, or a combination thereof) from a test biological sample processed as disclosed herein as an input, the algorithm can provide prognostic value(s) or treatment recommendation(s) to guide treatment decisions.

The algorithm can be used for an early stage cancer and can include a prognostic value or treatment recommendation related to, for example, administering a therapeutic, or not administering a therapeutic (e.g., because the tumor is classified as non-aggressive, and/or due to a lack of expected benefit).

### Example 17: Normalized Gene Expression Using Data from Multiple Sources, Discrimination of Clinical Biomarkers Status Based on Normalized Gene Expression Data, and Identification of Aberrantly Expressed Genes in Normal Adjacent Tumor Samples

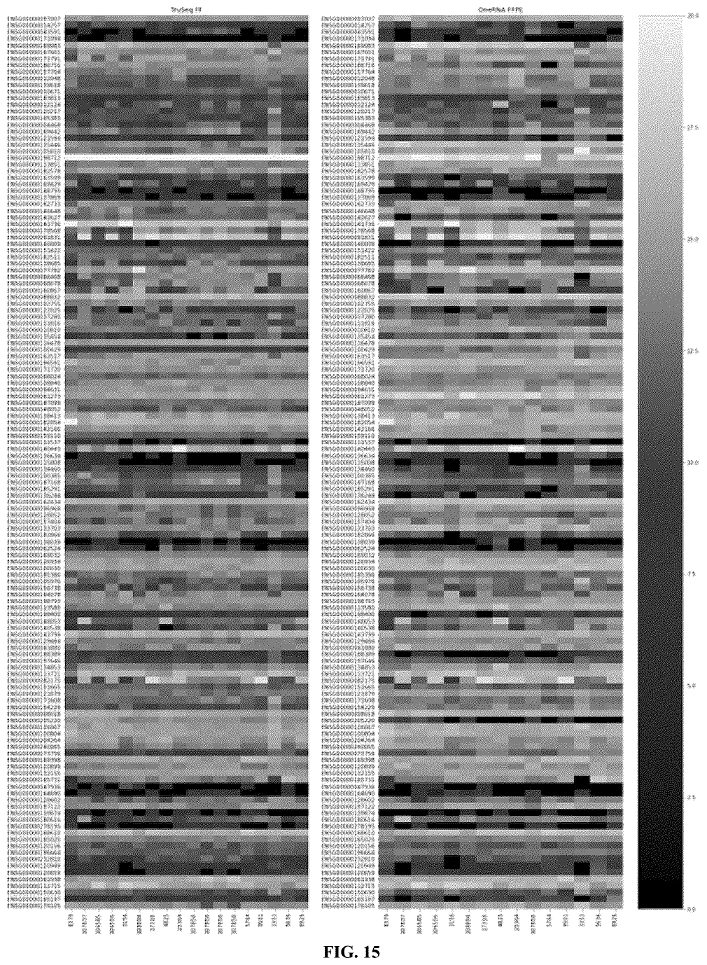

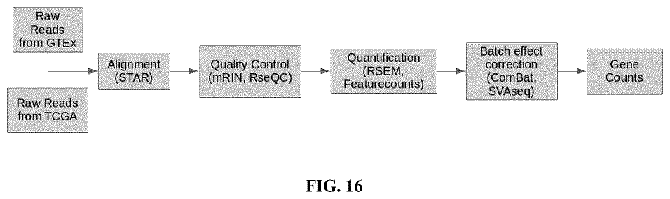

Batch-corrected maximum likelihood gene expression levels were obtained from data from The Cancer Genome Atlas Breast Invasive Carcinoma (TCGA) and The Genotype-Tissue Expression (GTEx) databases. Raw RNA sequencing reads from TCGA and GTEx projects were processed using a common bioinformatics pipeline (FIG. 16). The downloaded dataset was filtered for RSEM gene expression from breast samples. Sample information such as histological type and hormone receptor status was obtained from the Genomics Data Commons (GDC) for TCGA-BRCA data and GTEx Portal for GTEx samples. Samples were classified as three different tissue types: Tumor, Normal Adjacent Tissue (NAT) and Normal Tissue (NT). Tumor samples were samples in the TCGA dataset with the sample type “Primary Tumor”. NAT samples were also from the TCGA dataset with the sample type “Solid Tissue Normal”. From the TCGA protocol, NAT were collected >2 cm from tumor margin and/or contained no tumor by histopathologic review. Normal samples were from the GTEx dataset. Samples were filtered for those which were fresh frozen and from female donors. In total, 1,000 samples were used (109 NAT, 89 normal and 802 tumor).

Gene expression counts were normalized and aberrantly expressed genes detected as described in EXAMPLE 3. The data were filtered for genes included in the database of gene markers and targets associated with cancer therapeutics described in EXAMPLE 11.

Expression of three housekeeping genes (HKGs) was analyzed to evaluate the effect of normalization. UBC was used as a highly expressed HKG and has been used as a HKG to normalize between cancer cell lines. PUM1 was used as a gene with medium expression in breast tissue that was identified as a suitable HKG for study of breast cancer. NRF1 was used as a relatively weakly expressed gene with similar expression in healthy breast tissue, breast tumor, and NAT. Principal component analysis (PCA) was performed using the scikit-learn python module. Figures were generated using the plotline and matplotlib-pyplot python modules.

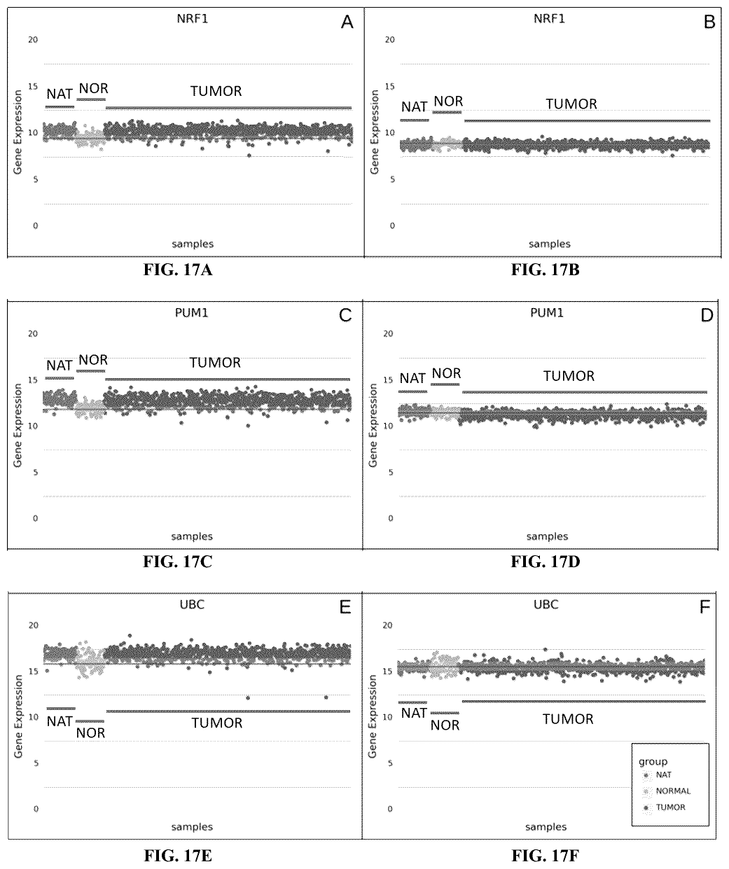

Prior to normalization, log-2 gene expression distribution showed clear separation based on data source (TCGA and GTEx) (FIGS. 17A, 17C, and 17E; samples are grouped by source—NAT: normal adjacent tissue from the TCGA dataset; NOR: normal control tissue from the GTEx dataset; TUMOR: primary tumor samples from the TCGA dataset). After normalization and scaling using the methods described in EXAMPLE 3, expression for HKGs was distributed randomly around the median with no clear distinction between the source datasets (FIGS. 17B, 17D, and 17F). This demonstrates that after normalization and correction for technical bias, HKG expression level was consistent between data sources and tissue types.

The normalized gene expression values were compared to clinical immunohistochemistry (IHC) data. Precision-recall curves were used to establish thresholds. For ER and PR IHC, the receptor status was considered positive if the sample displays >=10% cell positivity. Samples with <10% cell positivity were considered negative for ER and PR. Samples with a HER2 IHC score of 3+ were considered positive while scores of 1+ and 0 were labelled as negative following ASCO/CAP guidelines regarding HER2 testing in breast cancer. Scores with 2+ were not considered as they would be labelled as equivocal and require FISH testing to determine positivity. Tumor samples used in this analysis were split into training and testing sets. In total, 576 and 247 tumors were used as the training and testing sets, respectively. Precision-recall curves were calculated for each hormone receptor associated gene (ESR1, PGR, ERBB2) by iteratively changing the positivity threshold of normalized gene expression values with a step of 0.5, and comparing results to IHC results. Thresholds were determined by the highest f-score which was calculated using Equation 3 where R was chosen to be 0.5 such that recall is weighted lower than precision and will therefore maximize specificity.

$${F}_{β} = ( 1 + {β}^{2} ) * \frac{precision * recall}{( {β}^{2} * precision ) + recall} \qquad Equation 3$$

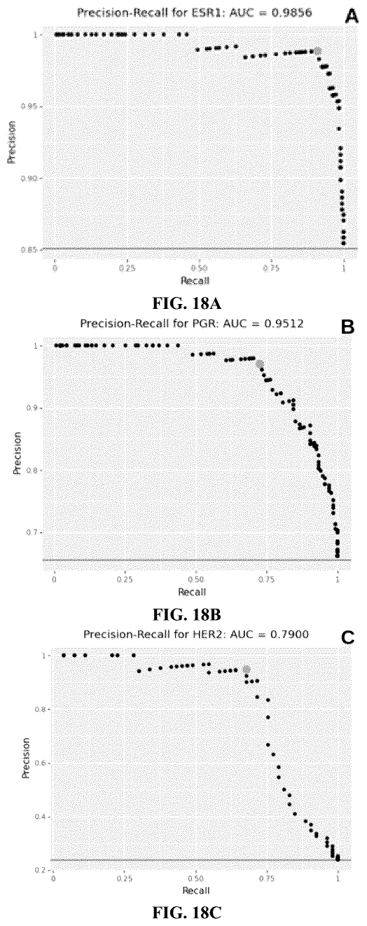

Precision-Recall was plotted using the training set to evaluate the ability of the normalized gene expression values to discriminate between positive and negative status for ESR1/ER (FIG. 18A), PGR/PR (FIG. 18B), and HER2 (FIG. 18C). AUC was calculated and all genes had an AUC score >=0.79. This indicates a high ability of the method to discriminate between positive and negative hormone receptor status according to the corresponding protein (IHC) data. Using the maximum f-score, thresholds were determined to predict IHC status (TABLE 4). Using the test dataset, the method was able to predict IHC hormone receptor status with high sensitivity and specificity (TABLE 5).

TABLE 4
TABLE 4: AUC, threshold, and threshold associated
F-score determined from precision recall curves for
ER, PR, and HER1 performed on training dataset.

| | AUC | Threshold | F-score |
|---|---|---|---|
| ESR1 | 0.986 | 11.000 | 0.972 |
| PGR | 0.951 | 10.300 | 0.909 |
| ERBB2/HER2 | 0.790 | 14.400 | 0.878 |

TABLE 5
TABLE 5: Performance characteristics in test dataset for predicting
ER, PR, and HER2 status using the thresholds set by training dataset
where gene expression below the threshold is a negative case while
expression above the threshold is a positive result for IHC. The
abbreviations tn, tp, fn, tp, tpr, tnr, ppv, and npv represent
true negatives, false positives, false negatives, true positives,
true positive rate, true negative rate, positive predictive value
and negative predictive value, respectively.

| | | | | | | tpr | tnr | | |
|---|---|---|---|---|---|---|---|---|---|
| | | | | | | (Sensi- | (Speci- | | |
| | Threshold | tn | fp | fn | tp | tivity) | ficity) | ppv | npv |
| ESR1 | 11 | 18 | 2 | 7 | 84 | 0.92 | 0.9 | 0.98 | 0.72 |
| PGR | 10.3 | 28 | 3 | 15 | 53 | 0.78 | 0.9 | 0.95 | 0.65 |
| ERBB2/ | 14.4 | 77 | 0 | 5 | 14 | 0.74 | 1 | 1 | 0.94 |
| HER2 | | | | | | | | | |

The results for ESR1, PGR, and ERBB2 were also used to predict IHC results for ER, PR, and HER2—respectively—in an experimental dataset. 15 breast tumor fresh frozen samples were sequenced and processed using a Genotype-Tissue Expression (GTEx) protocol. Library prep was performed using Illumina TruSeq Library Prep. Sequencing data was aligned and transcripts were quantified using RNAseqDB. For ER and PR, IHC results were able to be obtained for 10 samples; for HER2, 9 samples. IHC results for ER, PR, and HER2 were obtained from donor pathology reports and were considered positive if scored by the pathologist as positive, weakly positive, or equivocal. Samples were sequenced using RNA-seq protocols outlined in GTEx using the library preparation TruSeq for FF tissue. Sequence reads were aligned using the pipeline established in Wang et. al, “Unifying cancer and normal RNA sequencing data from different sources.” Scientific data 5.1 (2018): 1-8. Gene expression counts were normalized using the method described in EXAMPLE 3. IHC status for ER, PR, and HER2 were determined by using the thresholds set by the TCGA-BRCA samples. Samples were considered positive if normalized gene expression was greater in the corresponding gene.

IHC results were predicted using the thresholds set by the TCGA-BRCA training set (TABLE 6). TCGA-BRCA thresholds had perfect concordance with ER and PR IHC status. HER2 had one false negative, decreasing sensitivity.

TABLE 6
TABLE 6: Performance characteristics in sequenced fresh-frozen tumor
breast samples. The abbreviations tn, tp, fn, tp, tpr, tnr, ppv,
and npv represent true negatives, false positives, false negatives,
true positives, true positive rate, true negative rate, positive
predictive value and negative predictive value, respectively.

| | | | | | Tpr | Tnr | | |
|---|---|---|---|---|---|---|---|---|
| | | | | | (Sentiv- | (Speci- | | |
| | tn | fp | fn | tp | ity) | ficity) | ppv | npv |
| ESR1 | 3 | 0 | 0 | 7 | 1 | 1 | 1 | 1 |
| PGR | 4 | 0 | 0 | 6 | 1 | 1 | 1 | 1 |
| ERBB2/HER2 | 6 | 0 | 1 | 2 | 0.67 | 1 | 1 | 0.86 |

These methods demonstrate that methods disclosed herein can predict hormone receptor status based on the hormone receptor's associated transcript (e.g., ESR1, PGR, ERBB2) with relatively high accuracy. Hormone receptor status is an important aspect in breast cancer diagnosis and prognosis. However, current methods such as IHC and FISH are labor intensive, low throughout, expensive, and are typically performed for one biomarker at the time. RNA-sequencing has the ability to profile a large number of biomarkers on once.

Next, aberrantly expressed genes in normal adjacent tissue (NAT) were identified using thresholds set by GTEx normal tissue (NT). In some cases NATs are used as controls in cancer studies, however histologically normal tissue adjacent to tumors can contain molecular differences distinct from truly normal tissue (e.g., from control subjects without the tumor or without a diagnosed pathological condition).

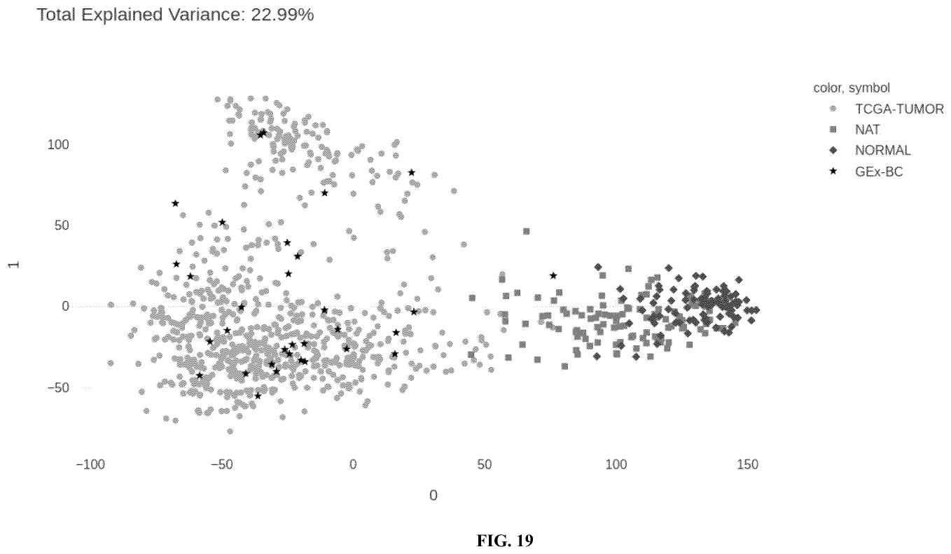

Principal Component Analysis (PCA) was done on normalized gene expression values (calculated by a method disclosed herein). Adjacent normal (NAT) samples and true normal breast samples were more similar to each other compared to tumor when plotted against the first and second principle component (FIG. 19). However, NATs overlap with tumor samples on the first and second principal component, suggesting similarities with tumor samples. In addition to data from the TCGA and Genotype-Tissue Expression (GTEx) databases, breast cancer samples newly sequenced in this example were also included in the analysis; these (labelled as GEx-BC) clustered with the TCGA-TUMOR samples.

NATs had a lower number of aberrantly expressed genes compared to tumor samples. However, NATs had a higher number of aberrantly expressed genes compared to normal samples (TABLE 7) and showed aberrant gene expression similar to tumor samples. These results combined with the PCA analysis of NAT compared to tumor and normal tissue suggests that NATs are neither normal nor tumor tissue. The ability of the method of the disclosure to detect differences between normal tissue and NAT could have applications in early detection of cancers or surveillance of remission.

TABLE 7
TABLE 7: Average number of aberrantly expressed
genes in NAT, tumor, and normal tissue.

| | Mean Number of LOW/ | Mean Number of HIGH/ |
|---|---|---|
| Sample Type | VERY LOW | VERY HIGH |
| Normal | 167.4 | 304.4 |
| Tumor | 2104.5 | 2552.2 |
| NAT | 1209.9 | 1151.2 |

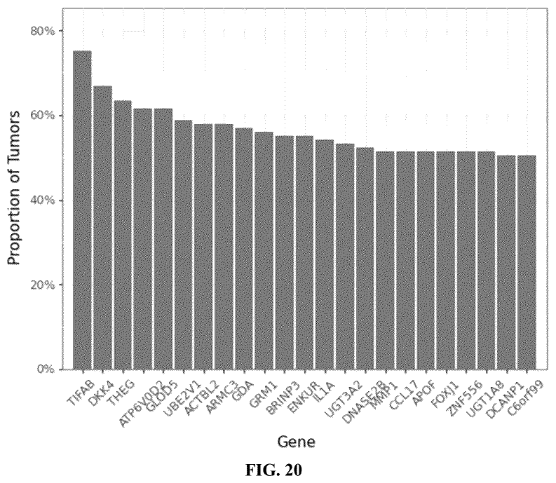

23 genes showed significant over-expression in >50% of NAT samples (categorized as VERY HIGH; FIG. 20). Of the 23 genes, presence or over-expression of many genes was found to be related to breast cancer. For example, THEG—also known as cancer/testis antigen 56—was found to be highly expressed in 63.3% of NAT and could represent a potential target for cancer immunotherapy or a cancer vaccine. Many highly expressed genes in NAT are also involved in modulating inflammatory response such as IL1A, GRM1, and UBE2V1. Inflammation can play a role in tumor progression and cancer risk and discovery of these inflammatory markers in NATs could have applications in the surveillance and assessment of cancer risk in women.

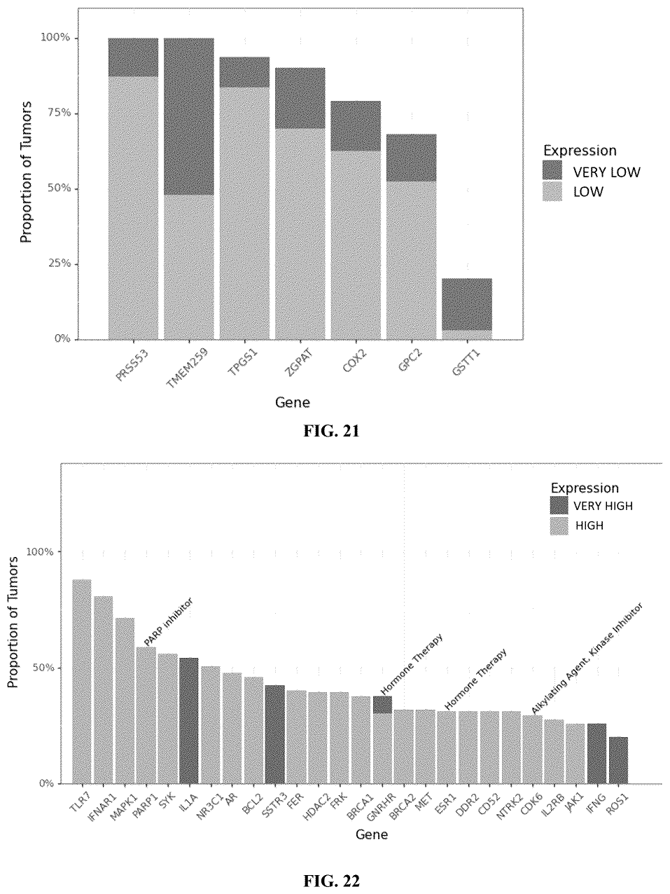

In >10% of NAT samples, 7 genes were found to be significantly under-expressed (FIG. 21). Of the 7 genes, decreased expression and null genotype of ZGPAT and GSTT1, respectively, was associated with increased breast cancer risk. ZGPAT has been demonstrated to inhibit cell proliferation through the regulation of EGFR. Homozygous deletion of GSTT1 has also been associated with an increase in breast cancer risk.

Additionally, some of the over-expressed genes found in NAT are targets for breast cancer therapeutics (FIG. 22; TABLE 8). In the context of NAT, these treatments have the potential of preventative care or early stage intervention. For example, >30% of NAT showed over-expression of the estrogen receptor gene ESR. The estrogen receptor is a therapeutic target for Tamoxifen which can be used to reduce the risk of breast cancer in healthy patients at increased risk of breast cancer.

TABLE 8
TABLE 8: Sample penetrance for genes that are over-expressed
in >20% of NAT samples and that are also targets
or markers for existing breast cancer treatments.

| GENE | HIGH | OVER | NCI name | Target | Type |
|---|---|---|---|---|---|
| GNRHR | 7.3% | 30.3% | Goserelin | Pituitary | direct |
| | | | Acetate | gonadotropin | |
| | | | | secretion | |
| ESR1 | | 31.2% | Tamoxifen | estrogen receptor | direct |
| | | | Citrate | | |
| ESR1 | | 31.2% | Toremifene | estrogen receptor | direct |
| ESR1 | | 31.2% | Anastrozole | estrogen receptor | direct |
| ESR1 | | 31.2% | Fulvestrant | estrogen receptor | direct |
| CDK6 | | 29.4% | Abemaciclib | CDK6 | direct |
| CDK6 | | 29.4% | Palbociclib | CDK6 | direct |
| CDK6 | | 29.4% | Ribociclib | CDK6 | direct |
| PARP1 | | 58.7% | Talazoparib | PARP1 | direct |
| | | | Tosylate | | |

These results demonstrate the ability of methods of the disclose to detect aberrant gene expression by comparing an individual's gene expression to normal tissue established thresholds. The method was able to accurately predict ER, PR and HER2 status in TCGA-BRCA tumor samples when compared to IHC results obtained from TCGA, as well as in a separate newly-sequenced experimental dataset. Such methods can allow RNA-sequencing to be used in addition to or in place of other clinically validated tests.

### Example 18: Identification of a Highly Expressed Gene in Metastatic Thyroid Cancer and a Suitable Corresponding Therapeutic

A tumor sample was collected from a subject with metastatic thyroid cancer. The sample was processed according to the methods of EXAMPLES 1-3 to generate normalized gene expression values. Expression of genes identified as relevant to cancer therapeutics in a database (e.g., genes that are markers or targets as described in EXAMPLE 11) was analyzed.

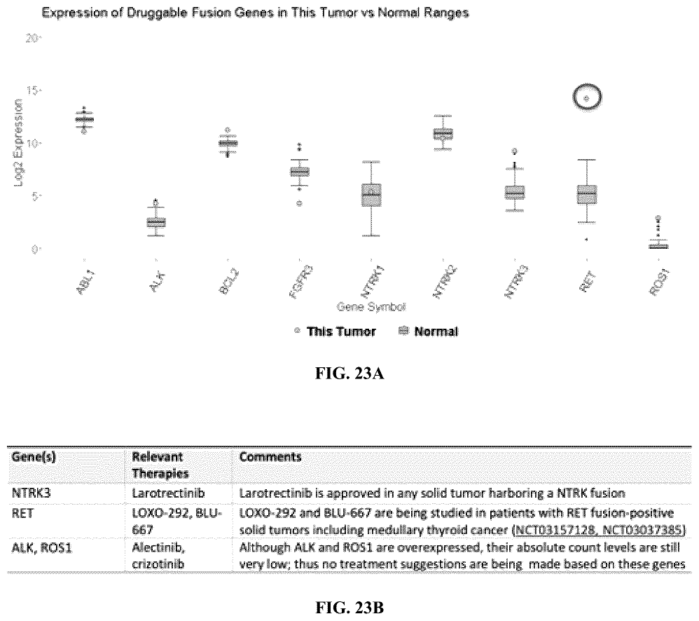

The normalized gene expression values and genes identified as relevant to cancer therapeutics were output into a report. The report included groups of aberrantly expressed genes based on mechanism and/or target category. Panels included homologous repair pathway genes, kinase target genes, immune checkpoint genes, hormone receptor genes, and fusion partners for drugs targeting gene fusions. The report comprised the information in FIG. 23A and FIG. 23B for fusion partners for drugs (e.g., approved drugs) targeting fusion genes The report included treatment recommendations based on categorization of expression (e.g., VERY LOW, LOW, NORMAL, HIGH, or VERY HIGH) and/or total/absolute expression counts.

Expression of RET was categorized as VERY HIGH, and corresponding clinical trials testing RET inhibitors were identified. Based on the finding and the report, the subject was enrolled in a clinical trial for the RET inhibitor selpercatinib. The subject responded to treatment and was in remission at follow up over two years later.

### Example 19: Comparison of Performance of Normalization Methods

Universal Human Reference RNA (UHRR) was fragmented to simulate various degree of RNA degradation. 200 μL of UHRR was prepared and 1 μL was taken out and diluted 1:10 before Qubit quantification. The undiluted concentration was quantified to 966.0 ng/μL. Of the remaining 199 μL, 49 μL was transferred to a tube marked “0s”, 50 μL was transferred to a tube marked “60s”, and 100 μL was transferred to a tube marked “720s”. The 50 μL from the “60s” tube was transferred to a Covaris microTUBE Screw-Cap for 50 μL samples marked “60s”. 50 μL from one of the tubes marked “720s” was transferred to a Covaris microTUBE Screw-Cap for 50 μL samples marked “720s”. The same microTUBE Screw-Cap tube was used twice to fragment the remaining 50 μL from the tube marked “720s”.

The Covaris microTUBE Screw-Cap for 50 μL samples were fragmented in a Covaris M220 Ultrasonicator with the following parameters: microTUBE AFA Fiber: Screw-Cap for 50 μL. Sample volume: 50 μL. Peak Incident Power: 50 W. Duty Factor: 20%. Cycles per Burst 200. Temperature 7° C.

20 μg in 20.7 μL (966.0 ng/μL) of either fragmented or unfragmented (“0s”) UHRR was treated with 9 μL BaseLine Zero DNase (BLZ) in a total volume of 180 μL including 18 μL of 10×BLZ Buffer. The two aliquots marked “720s” were digested with BLZ in two separate reactions, incubated at 37° C. for 30 min. No enzyme inactivation step was included, rather the samples were column purified directly after incubation.

All samples were purified using RNA Clean & Concentrator-5 columns from Zymo Research. The two aliquots marked “720s” were cleaned up on the same column in one processing. 2 volumes (360 μL) RNA Binding Buffer was added to the 180 μL BLZ reaction mix and mixed well. Equal volume (540 μL) of 100% ethanol was added and mixed well. Samples were transferred to Zymo-Spin IC columns in collection tubes and centrifuged. Flow through was discarded. 400 μL RNA Prep Buffer was added to the column, which was then centrifuged. Flow through was discarded. The column was washed twice with RNA Wash Buffer and centrifuged for 1 minute for removal of wash buffer from the binding matrix. Columns were transferred into a RNase-free tubes. 10 μL DNase/RNase-Free water was added directly to the column matrix, and the RNA was eluted by centrifugation. All centrifugation steps were at 10,000-16,000×g for 30 seconds.

1 μL of each purified product was taken and diluted 1:100 before Qubit quantification. The undiluted concentrations were quantified to: “0s”: 1.2 μg/μL; “60s”: 1.1 μg/μL; “720s”: 1.8 μg/μL.

Samples exhibited DV200 values of approximately 96.26% for the 0s condition (intact UHRR), 77.25% for the 60s condition (60s fragmented UHRR), and 27.77% for the 720s condition (720s fragmented UHRR), indicating increasing degrees of fragmentation (TABLES 9-11).

Sequencing libraries were generated in triplicate for the 0s, 60s, and 720s samples, with varied input amounts as follows. 0s libraries were generated using 50 ng or 500 ng of intact UHRR. 60s libraries were generated using 5 ng, 50 ng, or 500 ng of 60s fragmented UHRR. 720s libraries were generated using 50 ng or 500 ng of 720s fragmented UHRR. Equal volumes of each library were pooled, and the pool was sequenced on a MiSeq with a nano kit in order to assess the clustering efficiency of the individual libraries. A new pool for NextSeq sequencing was put together using the clustering efficiencies of the individual libraries on the MiSeq to adjust the volumes so as to obtain equal numbers of raw reads. The sequencing was carried out using a standard Illumina protocol.

The libraries were sequenced and processed to generate gene expression counts and compare different normalization strategies. Gene expression counts were deduplicated, then gene expression counts were normalized by: (i) the method described in EXAMPLE 3, (ii) a trimmed mean of M values (TMM) method using the tool EdgeR, or (iii) a Relative Log Expression (RLE) method using the tool DESeq2. R-squared values were calculated for the correlation of gene expression values between each pair of replicates in each condition (e.g., between each 0s replicate and every other 0s replicate, between each 60s replicate and every other 60s replicate, and between each 720s replicate and every other 720s replicate). As the RNA in all replicates originated from the same control source (UHRR), high positive correlations between replicates can be indicative of accurate data processing and normalization.

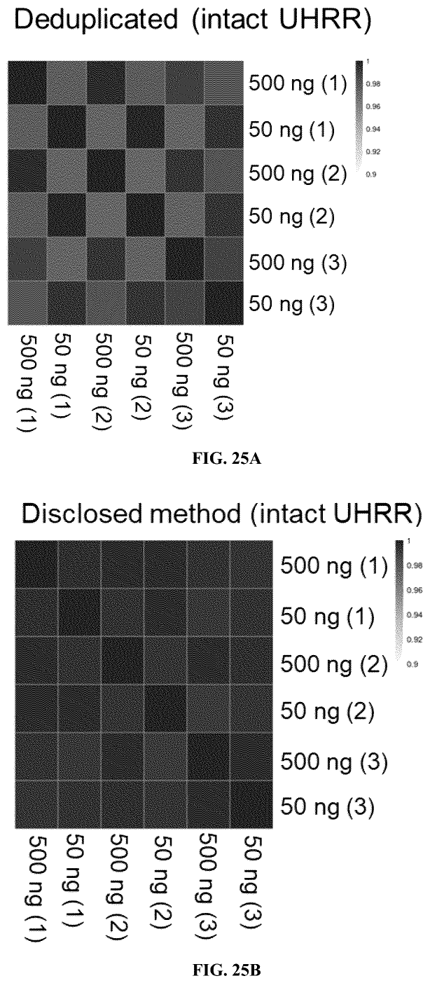

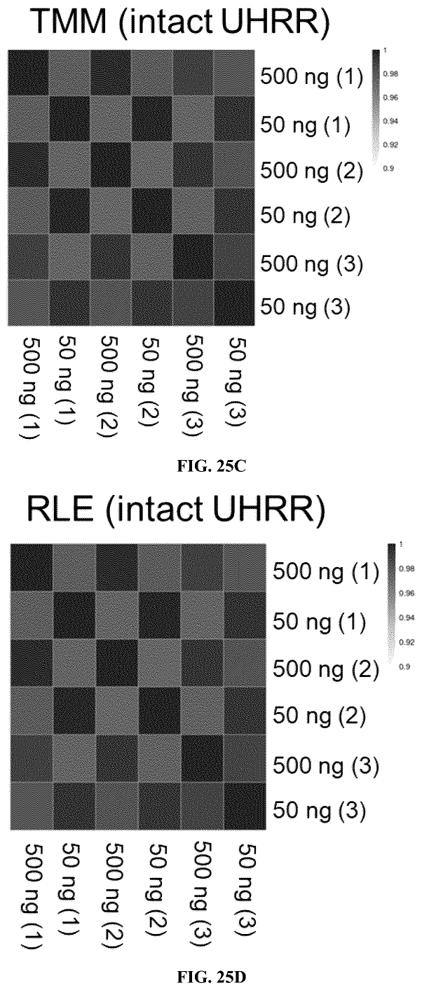

FIGS. 25A-27D show R-squared correlation values between replicates. Darker squares in the figures indicate a higher degree of correlation.

FIGS. 25A, 25B, 25C, and 25D illustrate correlations for the 0s samples after deduplication, deduplication plus normalization by the method disclosed herein, deduplication plus normalization by TMM, and deduplication plus normalization by RLE, respectively.

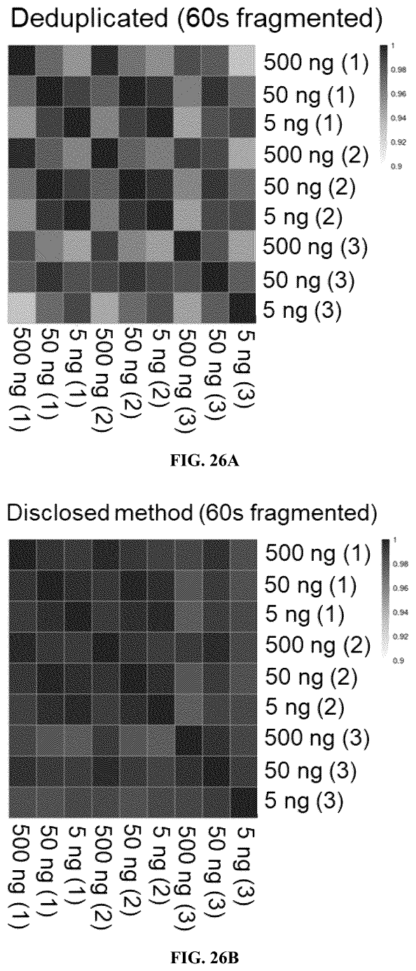

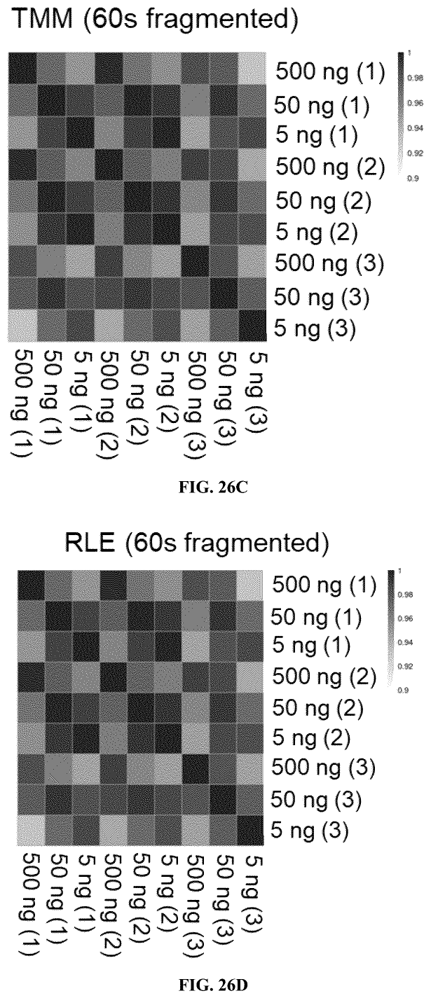

FIGS. 26A, 26B, 26C, and 26D illustrate correlations for the 60s samples after deduplication, deduplication plus normalization by the method disclosed herein, deduplication plus normalization by TMM, and deduplication plus normalization by RLE, respectively.

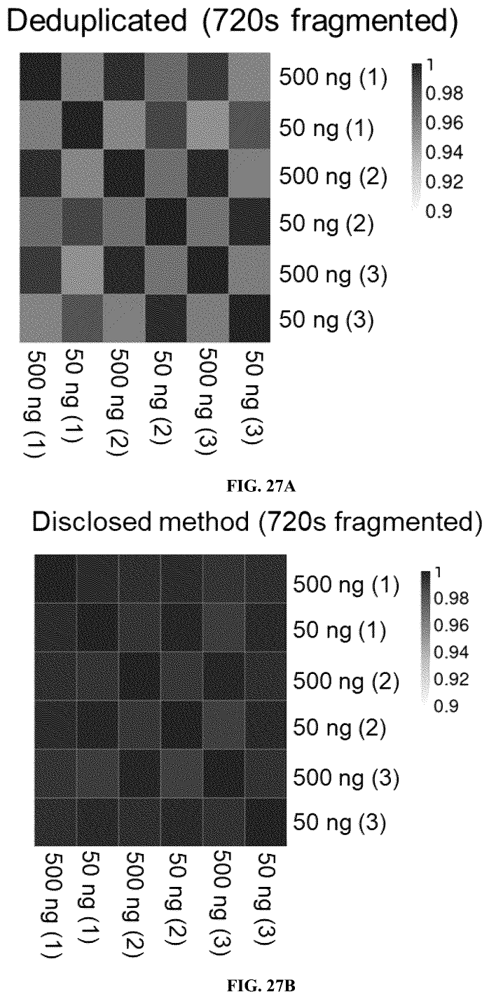

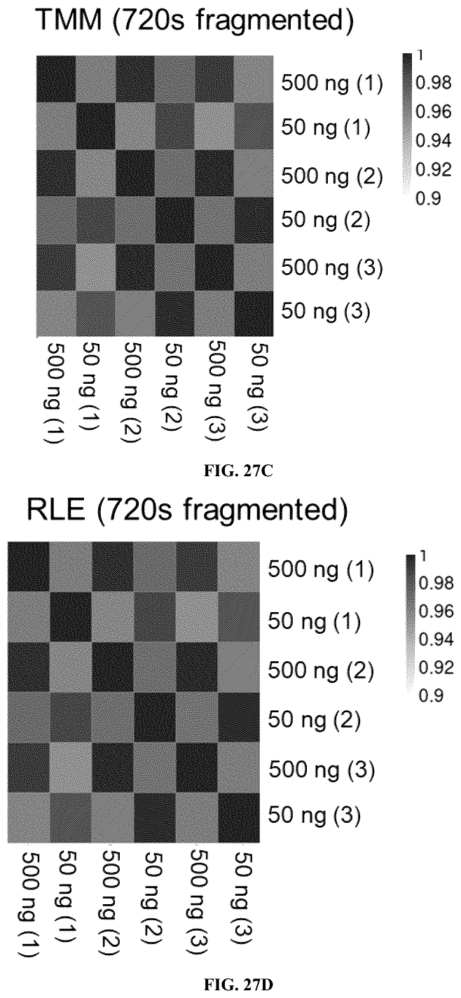

FIGS. 27A, 27B, 27C, and 27D illustrate correlations for the 720s samples after deduplication, deduplication plus normalization by the method disclosed herein, deduplication plus normalization by TMM, and deduplication plus normalization by RLE, respectively.

The normalization method disclosed herein provided a cross correlation of above 99% across the matrix, even for the highly fragmented RNA samples (FIG. 27B). In comparison, TMM and RLA did not improve or only minimally improved the cross correlation values compared to the subsampling, indicating that the normalization method disclosed herein out-performed the control techniques.

TABLE 9 provides details of RNA input amounts, DV200 values, and assigned reads before and after deduplication for the 0s samples.

TABLE 9

| RNA input | | | Total # of Raw | Total # of Assigned |
|---|---|---|---|---|
| amount | | DV 200 | Assigned Reads | Reads after Deduplication |
| 500 | ng (1) | 96.26% | 7,274,171 | 3,951,012 |
| 50 | ng (1) | 96.26% | 4,399,182 | 2,378,262 |
| 500 | ng (2) | 96.26% | 9,694,045 | 4,721,084 |
| 50 | ng (2) | 96.26% | 6,855,009 | 2,670,276.00 |
| 500 | ng (3) | 96.26% | 6,160,540 | 3,139,229 |
| 50 | ng (3) | 96.26% | 7,275,672 | 3,339,995 |

TABLE 10

| RNA input | | | Total # of Raw | Total # of Assigned |
|---|---|---|---|---|
| amount | | DV 200 | Assigned Reads | Reads after Deduplication |
| 500 | ng (1) | 77.25% | 6,068,142 | 3,343,103 |
| 50 | ng (1) | 77.25% | 5,309,507 | 2,200,348 |
| 5 | ng (1) | 77.25% | 5,327,034 | 1,235,341 |
| 500 | ng (2) | 77.25% | 5,946,418 | 3,385,438 |
| 50 | ng (2) | 77.25% | 3,079,491 | 1,820,123 |
| 50 | ng (2) | 77.25% | 5,173,302 | 1,294,805 |
| 500 | ng (3) | 77.25% | 3,300,106 | 2,128,278 |
| 50 | ng (3) | 77.25% | 4,161,297 | 2,424,186 |
| 5 | ng (3) | 77.25% | 648,802 | 456,378 |

TABLE 11

| RNA input | | | Total # of Raw | Total # of Assigned |
|---|---|---|---|---|
| amount | | DV 200 | Assigned Reads | Reads after Deduplication |
| 500 | ng (1) | 27.77% | 5,722,193 | 3,227,237 |
| 50 | ng (1) | 27.77% | 5,309,507 | 2,200,348 |
| 500 | ng (2) | 27.77% | 6,113,445 | 3,485,441 |
| 50 | ng (2) | 27.77% | 5,798,759 | 2,308,520 |
| 500 | ng (3) | 27.77% | 6,680,016 | 3,366,866 |
| 50 | ng (3) | 27.77% | 6,675,037 | 2,406,091 |

1-285. (canceled)

286. A method comprising:
(a) processing gene expression counts of a test biological sample obtained from a test subject to obtain normalized gene expression values suitable for comparison to a database, wherein:
the gene expression counts are generated by RNA sequencing of the test biological sample obtained from the test subject;
the database comprises gene expression counts obtained from a plurality of control biological samples; and
wherein each of the control biological samples is a sample type that is comparable to the test biological sample, and each of the control biological samples is independently obtained from a normal control subject;
(b) identifying a gene that is aberrantly expressed in the test biological sample relative to the plurality of control biological samples; and
(c) providing a wellness recommendation based on the gene that is aberrantly expressed in the test biological sample relative to the plurality of control biological samples.

287. The method of claim 286, further comprising identifying at least a second gene that is aberrantly expressed in the test biological sample relative to the plurality of control biological samples.

288. The method of claim 286, wherein the gene that is aberrantly expressed in the test biological sample relative to the plurality of control biological samples is a drug target.

289. The method of claim 286, further comprising identifying a clinical trial in which the gene that is aberrantly expressed in the test biological sample relative to the plurality of control biological samples is a therapeutic target.

290. The method of claim 286, wherein the gene that is aberrantly expressed in the test biological sample relative to the plurality of control biological samples encodes an immune modulatory protein.

291. The method of claim 286, wherein the gene that is aberrantly expressed in the test biological sample relative to the plurality of control biological samples exhibits higher expression in the test biological sample than the plurality of control biological samples.

292. The method of claim 286, wherein the gene that is aberrantly expressed in the test biological sample relative to the plurality of control biological samples exhibits lower expression in the test biological sample than the plurality of control biological samples.

293. The method of claim 286, wherein a second database containing a group of genes that are associated with treatment responses is used to determine whether the gene that is aberrantly expressed in the test biological sample relative to the plurality of control biological samples is associated with a treatment response for a disease.

294. The method of claim 286, further comprising generating a report, wherein the report identifies the gene that is aberrantly expressed in the test biological sample relative to the plurality of control biological samples.

295. The method of claim 286, wherein the wellness recommendation comprises a recommendation of administering a therapeutic agent to the test subject based on an expression level of the gene that is aberrantly expressed in the test biological sample relative to the plurality of control biological samples.

296. The method of claim 286, further comprising identifying a therapeutic agent that modulates activity of a product encoded by the gene that is aberrantly expressed in the test biological sample relative to the plurality of control biological samples.

297. The method of claim 286, wherein the database comprises gene expression counts obtained from at least 10 control biological samples.

298. The method of claim 286, wherein the gene that is aberrantly expressed in the test biological sample relative to the plurality of control biological samples is identified by comparing the normalized gene expression values of the test biological sample to normalized gene expression values of the plurality of control biological samples.

299. The method of claim 286, wherein the test biological sample and each of the control biological samples comprise tissue samples of a same tissue type.

300. The method of claim 286, wherein the processing further comprises removing duplicate reads identified as originating from a same RNA molecule.

301. The method of claim 286, wherein the gene that is aberrantly expressed in the test biological sample relative to the plurality of control biological samples is suitable for inclusion in a cancer vaccine.

302. The method of claim 286, further comprising developing a therapeutic agent targeting the aberrantly expressed gene.

303. The method of claim 286, further comprising using an algorithm to identify an association between one or more of the normalized gene expression values and a clinical outcome associated with administering a therapeutic agent.

304. A method comprising processing gene expression counts of a test biological sample to obtain normalized gene expression values suitable for comparison to a database, wherein the database comprises gene expression counts from a plurality of control biological samples, wherein:
(a) the gene expression counts of the test biological sample are:
(i) generated by RNA sequencing of the test biological sample;
(ii) subsampled to a target number of assigned reads; and
(iii) sorted by a total of gene expression counts assigned to each gene, thereby generating sorted gene expression counts of the test biological sample;
(b) the gene expression counts of each control biological sample of the plurality are:
(i) generated by RNA sequencing of the control biological sample;
(ii) subsampled to the target number of assigned reads; and
(iii) sorted by a total of gene expression counts assigned to each gene, thereby generating sorted gene expression counts of the control biological sample; and
(c) the processing comprises, for each position of the sorted gene expression counts of the test biological sample, calculating a normalized gene expression value from an average of:
(i) gene expression count at the position of the sorted gene expression counts of the test biological sample; and
(ii) gene expression count for each of the plurality of control biological samples at a corresponding position of the sorted gene expression counts of the control biological sample;
thereby generating the normalized gene expression values suitable for comparison to the database.

305. A computer program product comprising a non-transitory computer-readable medium having computer-executable code encoded therein, the computer-executable code adapted to be executed to implement a method, the method comprising:
a) running a gene processing system, wherein the gene processing system comprises:
i) an expression count processing component;
ii) a gene identifying component;
iii) a recommendation component;
iv) a database of gene expression counts obtained from a plurality of control biological samples, wherein each of the control biological samples is a sample type that is comparable to a test biological sample, and each of the control biological samples is independently obtained from a normal control subject; and
v) an output component;
b) processing, by the expression count processing component, gene expression counts of RNA sequencing of the test biological sample obtained from a test subject to obtain gene expression values suitable for comparison to the database;
c) identifying, by the gene identifying component, a gene that is aberrantly expressed in the test biological sample relative to the plurality of control biological samples;
d) providing a wellness recommendation, by the recommendation component, based on the gene that is aberrantly expressed in the test biological sample relative to the plurality of control biological samples; and
e) outputting, by the output component, a report that comprises the wellness recommendation.
